(19) United States
(12) Patent Application Publication
(10) Pub. No.: US 2013/0195870 A1
(43) Pub. Date: Aug. 1, 2013

(54) ERBB3 Mutations In Cancer

(71) Applicant: Genentech, Inc., South San Francisco, CA (US)

(72) Inventors: Bijay Shankar Jaiswal, South San Francisco, CA (US); Somasekar Seshagiri, South San Francisco, CA (US)

(73) Assignee: Genentech, Inc., South San Francisco, CA (US)

(21) Appl. No.: 13/694,413

(22) Filed: Nov. 29, 2012

Related U.S. Application Data
(60) Provisional application No. 61/629,951, filed on Nov. 30, 2011.

(51) Int. Cl. C12Q 1/68; A61K 39/395; A61K 31/517

(52) U.S. Cl. CPC C12Q 1/6886; A61K 31/517; A61K 39/39558
USPC 424/136.1; 536/24.31; 435/6.11; 514/266.24; 424/139.1; 424/133.1; 506/9

(57) ABSTRACT

The present invention concerns somatic ErbB3 mutations in cancer including methods of identifying, diagnosing, and prognosing ErbB3 cancers, as well as methods of treating cancer, including certain subpopulations of patients.

### RELATED APPLICATIONS

This application claims priority to under 35 U.S.C. §119(e) and the benefit of U.S. Provisional Application Ser. No. 61/629,951 filed on Nov. 30, 2011, which is incorporated by reference herein in its entirety.

### SEQUENCE LISTING

The instant application contains a Sequence Listing which has been submitted in ASCII format via EFS-Web and is hereby incorporated by reference in its entirety. Said ASCII copy, created on Mar. 29, 2013, is named GNE391US.txt and is 177,443 bytes in size.

### FIELD OF THE INVENTION

The present invention concerns somatic ErbB3 mutations in cancer including methods of identifying, diagnosing, and prognosing ErbB3 cancers, as well as methods of treating cancer, including certain subpopulations of patients.

### BACKGROUND OF THE INVENTION

The human epidermal growth factor receptor (HER) family of receptor tyrosine kinases (RTK), also known as ERBB receptors, consists of four members: EGFR/ERBB1/HER1, ERBB2/HER2, ERBB3/HER3 and ERBB4/HER4 (Hynes et al. Nature Reviews Cancer 5, 341-354 (2005); Baselga et al. Nature Reviews Cancer 9, 463-475 (2009)). The ERBB family members contain an extracellular domain (ECD), a single-span transmembrane region, an intracellular tyrosine kinase domain, and a C-terminal signaling tail (Burgess et al. Mol Cell 12, 541-552 (2003); Ferguson. Annual Review of Biophysics 37, 353-373 (2008)). The ECD is a four domain structure consisting of two L domains (I and III) and two cysteine-rich domains (II and IV) (Burgess et al. Mol Cell 12, 541-552 (2003); Ferguson. Annual Review of Biophysics 37, 353-373 (2008)). The ERBB receptors are activated by multiple ligands that include epidermal growth factor (EGF), transforming growth factor-α (TGF-α) and neuregulins (Yarden et al. Nat Rev Mol Cell Biol 2, 127-137 (2001)). Activation of the receptor involves a single ligand molecule binding simultaneously to domains I and III, leading to heterodimerization or homodimerization through a dimerization arm in domain II (Burgess et al. Mol Cell 12, 541-552 (2003); Ogiso et al. Cell 110, 775-787 (2002); Cho. Science 297, 1330-1333 (2002); Dawson et al. Molecular and Cellular Biology 25, 7734-7742 (2005); Alvarado et al. Cell 142, 568-579 (2010); Lemmon et al. Cell 141, 1117-1134 (2010)). In the absence of ligand, the domain II dimerization arm is tucked away via an intramolecular interaction with domain IV, leading to a “tethered”, auto-inhibited configuration (Burgess et al. Mol Cell 12, 541-552 (2003); Cho. Science 297, 1330-1333 (2002); Lemmon et al. Cell 141, 1117-1134 (2010); Ferguson et al. Mol Cell 11, 507-517 (2003)).

Although the four ERBB receptors share a similar domain organization, functional and structural studies show that ERBB2 does not bind any of the known ERBB family ligands and is constitutively in an “untethered” (open) conformation suitable for dimerization (Garrett et al. Mol Cell 11, 495-505 (2003). In contrast, ERBB3, though capable of ligand binding, heterodimerzation and signaling, has an impaired kinase domain (Baselga et al. Nature Reviews Cancer 9, 463-475 (2009); Jura et al. Proceedings of the National Academy of Sciences 106, 21608-21613 (2009); Shi et al. Proceedings of the National Academy of Sciences 107, 7692-7697 (2010). Although, ERBB2 and ERBB3 are functionally incomplete on their own, their heterodimers are potent activators of cellular signaling (Pinkas-Kramarski et al. The EMBO Journal 15, 2452-2467 (1996); Tzahar et al. Molecular and Cellular Biology 16, 5276-5287 (1996); Holbro et al. Proceedings of the National Academy of Sciences 100, 8933-8938 (2003)).

While the ERBB receptors are critical regulators of normal growth and development, their deregulation has also been implicated in development and progression of cancers (Baselga et al. Nature Reviews Cancer 9, 463-475 (2009); Sithanandam et al. Cancer Gene Ther 15, 413-448 (2008); Hynes et al. Current Opinion in Cell Biology 21, 177-184 (2009)). In particular, gene amplification leading to receptor overexpression and activating somatic mutations are known to occur in ERBB2 and EGFR in various cancers (Sithanandam et al. Cancer Gene Ther 15, 413-448 (2008); Hynes et al. Current Opinion in Cell Biology 21, 177-184 (2009); Wang et al. Cancer Cell 10, 25-38 (2006); Yamauchi et al. Biomark Med 3, 139-151 (2009)). This has led to the development of multiple small molecule and antibody based therapeutics that target EGFR and ERBB2 (Baselga et al. Nature Reviews Cancer 9, 463-475 (2009); Alvarez et al. Journal of Clinical Oncology 28, 3366-3379 (2010)). Although the precise role of ERBB4 in oncogenesis is not well established (Koutras et al. Critical Reviews in Oncology/Hematology 74, 73-78 (2010)), transforming somatic mutations in ERBB4 have been reported in melanoma (Prickett et al. Nature Genetics 41, 1127-1132 (2009)). Recently, ERBB3 has emerged as a potential cancer therapeutic target, given that it plays an important role in ERBB2 signaling and is also implicated in promoting resistance to existing therapeutics (Baselga et al. Nature Reviews Cancer 9, 463-475 (2009); Amin et al. Semin Cell Dev Biol 21, 944-950 (2010)). While ERBB3 amplification and/or overexpression is known in some cancers, only sporadic occurrence of ERBB3 somatic mutations has been reported, although the functional relevance of these mutations has not been studied. The invention provided herein concerns the identification of frequent ERBB3 somatic mutations in human cancers.

### SUMMARY OF THE INVENTION

The present invention is based at least in part on the discovery of multiple somatic mutational events in the ERBB3 receptor of the human epidermal growth factor receptor (HER) family of receptor tyrosine kinases (RTK), that are associated with various human tumors including, without limitation, gastric and colon tumors. It is believed that these mutations predispose and/or directly contribute to human tumorigenesis. Indeed, as described herein, there is evidence that some of the mutations promote oncogenesis in vivo.

In one aspect, the present invention provides ErbB3 cancer detecting agents. In one embodiment, the ErbB3 cancer detecting agent is an ErbB3 gastrointestinal cancer detecting agent. In another embodiment, the detecting agent comprises a reagent capable of specifically binding to an ErbB3 mutation in an ErbB3 nucleic acid sequence. In one other embodiment, the ErbB3 nucleic acid sequence comprises SEQ ID NO:3 or 1.

In some embodiments, the reagent comprises a polynucleotide of formula

5′Xₐ—Y—Z_{b}3′ Formula I,

wherein

X is any nucleic acid and a is between about 0 and about 250;

Y is an ErbB3 mutation codon; and

Z is any nucleic acid and b is between about 0 and about 250.

In one other embodiment, the mutation codon encodes (i) an amino acid at a position of SEQ ID NO:2 selected from the group consisting of 104, 809, 232, 262, 284, 325, 846, 928, 60, 111, 135, 295, 406, 453, 498, 1089, and 1164; or (ii) a stop codon at position 193. In one other embodiment, the gastrointestinal cancer is gastric cancer or colon cancer.

In another aspect, the present invention provides a method of determining the presence of ErbB3 gastrointestinal cancer in a subject. In one embodiment, the method comprises detecting in a biological sample obtained from the subject a mutation in a nucleic acid sequence encoding ErbB3, wherein the mutation results in an amino acid change at at least one position of the ErbB3 amino acid sequence and wherein the mutation is indicative of an ErbB3 gastrointestinal cancer in the subject. In another embodiment, the mutation resulting in an amino acid change is at a position of SEQ ID NO:2 selected from the group consisting of 104, 809, 232, 262, 284, 325, 846, 928, 60, 111, 135, 295, 406, 453, 498, 1089, 1164, and 193. In other embodiments, the gastrointestinal cancer is gastric cancer or colon cancer.

In another aspect, the present invention provides a method of determining the presence of ErbB3 cancer in a subject. In one embodiment, the method comprises detecting in a biological sample obtained from the subject the presence or absence of an amino acid mutation in a nucleic acid sequence encoding ErbB3, wherein the mutation results in an amino acid change at at least one position in SEQ ID NO: 2 selected from the group consisting of 104, 809, 232, 262, 284, 325, 846, 928, 60, 111, 135, 295, 406, 453, 498, 1089, 1164, 193, 492, and 714, and wherein the presence of the mutation is indicative of an ErbB3 cancer in the subject. In another embodiment, the ErbB3 cancer is selected from the group consisting of gastric, colon, esophageal, rectal, cecum, non-small-cell lung (NSCLC) adenocarinoma, NSCLC (Squamous carcinoma), renal carcinoma, melanoma, ovarian, lung large cell, small-cell lung cancer (SCLC), hepatocellular (HCC), lung, and pancreatic.

In yet another aspect, the determining methods further comprise one of the following additional steps: administering a therapeutic agent to said subject, identifying the subject in need, obtaining the sample from a subject in need, or any combination thereof. In one embodiment, the therapeutic agent is an ErbB inhibitor. In other embodiments, the ErbB inhibitor is selected from the group consisting of an EGFR antagonist, an ErbB2 antagonist, an ErbB3 antagonist, an ErbB4 antagonist, and an EGFR/ErbB3 antagonist. In another embodiment, the inhibitor is a small molecule inhibitor. In one embodiment, the antagonist is an antagonist antibody. In yet another embodiment, the antibody is selected from the group consisting of a monoclonal antibody, a bispecific antibody, a chimeric antibody, a human antibody, a humanized antibody and an antibody fragment.

In another aspect, the detecting step comprises amplifying or sequencing. In one embodiment, the detecting comprises amplifying or sequencing the mutation and detecting the mutation or sequence thereof. In another embodiment, the amplifying comprises admixing an amplification primer or amplification primer pair with a nucleic acid template isolated from the sample. In other embodiments, the primer or primer pair is complementary or partially complementary to a region proximal to or including said mutation, and is capable of initiating nucleic acid polymerization by a polymerase on the nucleic acid template. In one other embodiment, the amplifying further comprises extending the primer or primer pair in a DNA polymerization reaction comprising a polymerase and the template nucleic acid to generate an amplicon. In another embodiment, in the amplifying or sequencing, the mutation is detected by a process that includes one or more of: sequencing the mutation in a genomic DNA isolated from the biological sample, hybridizing the mutation or an amplicon thereof to an array, digesting the mutation or an amplicon thereof with a restriction enzyme, or real-time PCR amplification of the mutation. In yet another embodiment, the amplifying or sequencing further comprises partially or fully sequencing the mutation in a nucleic acid isolated from the biological sample. In other embodiments, the amplifying comprises performing a polymerase chain reaction (PCR), reverse transcriptase PCR (RT-PCR), or ligase chain reaction (LCR) using a nucleic acid isolated from the biological sample as a template in the PCR, RT-PCR, or LCR.

In one other aspect, the present invention provides a method of treating gastrointestinal cancer in a subject in need. In one embodiment, the method comprises a) detecting in a biological sample obtained from the subject a mutation in a nucleic acid sequence encoding ErbB3, wherein the mutation results in an amino acid change at at least one position of the ErbB3 amino acid sequence and wherein the mutation is indicative of an ErbB3 gastrointestinal cancer in the subject. In another embodiment, the method further comprises b) administering a therapeutic agent to said subject. In other embodiments, the mutation resulting in an amino acid change is at a position of SEQ ID NO:2 selected from the group consisting of 104, 809, 232, 262, 284, 325, 846, 928, 60, 111, 135, 295, 406, 453, 498, 1089, 1164, and 193. In another embodiment, the gastrointestinal cancer is gastric cancer or colon cancer.

In one aspect, the present invention provides a method of treating an ErbB3 cancer in a subject. In one embodiment, the method comprises of a) detecting in a biological sample obtained from the subject the presence or absence of an amino acid mutation in a nucleic acid sequence encoding ErbB3, wherein the mutation results in an amino acid change at at least one position in SEQ ID NO: 2 selected from the group consisting of 104, 809, 232, 262, 284, 325, 846, 928, 60, 111, 135, 295, 406, 453, 498, 1089, 1164, 193, 492, and 714, and wherein the presence of the mutation is indicative of an ErbB3 cancer in the subject. In another embodiment, the method further comprises b) administering a therapeutic agent to said subject. In some embodiments, the ErbB3 cancer is selected from the group consisting of gastric, colon, esophageal, rectal, cecum, colorectal, non-small-cell lung (NSCLC) adenocarinoma, NSCLC (Squamous carcinoma), renal carcinoma, melanoma, ovarian, lung large cell, small-cell lung cancer (SCLC), hepatocellular (HCC), lung, and pancreatic.

In another aspect, the methods of treatment involve ErbB3 inhibitors. In one additional embodiment, the therapeutic agent is an ErbB inhibitor. In another embodiment, the ErbB inhibitor is selected from the group consisting of an EGFR antagonist, an ErbB2 antagonist, an ErbB3 antagonist, an ErbB4 antagonist, and an EGFR/ErbB3 antagonist. In yet another embodiment, the antagonist is a small molecule inhibitor. In one embodiment, the antagonist is an antagonist antibody. In other embodiments, the antibody is selected from the group consisting of a monoclonal antibody, a bispecific antibody, a chimeric antibody, a human antibody, a humanized antibody and an antibody fragment.

### Additional Embodiments

In one aspect, the present invention provides methods of determining the presence of ErbB3 cancer in a subject in need. In one embodiment, the method comprises the step of detecting in a biological sample obtained from the subject the presence or absence of an amino acid mutation in a nucleic acid sequence encoding ErbB3, wherein the mutation results in an amino acid change at at least one position selected from the group consisting of M60, R193, A232, P262, V295, G325, M406, D492, V714, Q809, R1089, T1164. In another embodiment, the method further comprises administering a therapeutic agent to the subject. In one other embodiment, the method further comprises identifying the subject in need. In yet another embodiment, the method further comprises obtaining the sample from a subject in need. In one embodiment, the ErbB3 cancer is selected from the group consisting of gastric, colon, esophageal, rectal, cecum, non-small-cell lung (NSCLC) adenocarinoma, NSCLC (Squamous carcinoma), renal carcinoma, melanoma, ovarian, lung large cell, small-cell lung cancer (SCLC), hepatocellular (HCC), lung, and pancreatic.

In another aspect, the present invention provides methods of determining the presence of ErbB3 gastrointestinal cancer in a subject in need comprising detecting in a biological sample obtained from the subject a mutation in a nucleic acid sequence encoding ErbB3, wherein the mutation results in an amino acid change at at least one position selected from the group consisting of V104, Y111, A232, P262, G284, T389, and Q809. In another embodiment, the method further comprises administering a therapeutic agent to the subject. In one other embodiment, the method further comprises identifying the subject in need. In yet another embodiment, the method further comprises obtaining the sample from a subject in need. In one other embodiment, the ErbB3 gastrointestinal cancer is gastric cancer or colon cancer.

In one other aspect, the present invention provides methods of identifying ErbB3 gastrointestinal cancer in a subject in need that is likely to respond to an ErbB antagonist, said method comprising detecting in a gastrointestinal cancer cell obtained from the subject a mutation in a nucleic acid sequence encoding ErbB3, wherein the mutation at at least one position selected from the group consisting of V104, Y111, A232, P262, G284, T389, and Q809. In another embodiment, the method further comprises administering a therapeutic agent to the subject. In one other embodiment, the method further comprises obtaining the sample from a subject in need. In one other embodiment, the ErbB3 gastrointestinal cancer is gastric cancer or colon cancer.

In another aspect, the present invention provides methods of treating ErbB3 cancer in a subject in need. In one embodiment, the method comprises the step of detecting in a biological sample obtained from the subject the presence or absence of an amino acid mutation in a nucleic acid sequence encoding ErbB3, wherein the mutation results in an amino acid change at at least one position selected from the group consisting of M60, R193, A232, P262, V295, G325, M406, D492, V714, Q809, R1089, T1164. In another embodiment, the method further comprises the step of administering a therapeutic agent to said subject.

In another aspect, the present invention provides methods of treating ErbB3 gastrointestinal cancer in a subject in need. In one embodiment, the method comprises the step of detecting in a biological sample obtained from the subject a mutation in a nucleic acid sequence encoding ErbB3, wherein the mutation results in an amino acid change at at least one position selected from the group consisting of V104, Y111, A232, P262, G284, T389, and Q809. In another embodiment, the method further comprises the step of administering a therapeutic agent to said subject.

In one embodiment, the therapeutic agent administered in the methods of the present invention is an ErbB inhibitor. In another embodiment, the ErbB inhibitor is selected from the group consisting of an EGFR antagonist, an ErbB2 antagonist, an ErbB3 antagonist, an

ErbB4 antagonist, and an EGFR/ErbB3 antagonist. In one other embodiment, the inhibitor is a small molecule inhibitor. In some embodiments, the ErbB inhibitor is an EGFR antagonist. In other embodiments, the ErbB inhibitor is an ErbB2 antagonist. In one other embodiment, the ErbB inhibitor is an ErbB3 antagonist. In another embodiment, the ErbB inhibitor is an ErbB4 antagonist. In some embodiments, the ErbB inhibitor is an EGFR/ErbB3 antagonist. In other embodiments, the antagonist is an antagonist antibody. In some embodiments, the antibody is selected from the group consisting of a monoclonal antibody, a bispecific antibody, a chimeric antibody, a human antibody, a humanized antibody and an antibody fragment.

In another aspect, the methods of the present invention comprise a detecting step in which the nucleic acid sequence obtained from the sample is analyzed for the presence or absence of the mutation(s). In one embodiment, the detecting comprises amplifying or sequencing the mutation and detecting the mutation or sequence thereof. In another embodiment, the amplifying comprises admixing an amplification primer or amplification primer pair with a nucleic acid template isolated from the sample. In one other embodiment, the primer or primer pair is complementary or partially complementary to a region proximal to or including said mutation, and is capable of initiating nucleic acid polymerization by a polymerase on the nucleic acid template. In yet another embodiment, the method further comprises extending the primer or primer pair in a DNA polymerization reaction comprising a polymerase and the template nucleic acid to generate an amplicon. In some embodiments, the mutation is detected by a process that includes one or more of: sequencing the mutation in a genomic DNA isolated from the biological sample, hybridizing the mutation or an amplicon thereof to an array, digesting the mutation or an amplicon thereof with a restriction enzyme, or real-time PCR amplification of the mutation. In other embodiments, the method comprises partially or fully sequencing the mutation in a nucleic acid isolated from the biological sample. In one embodiment, the amplifying comprises performing a polymerase chain reaction (PCR), reverse transcriptase PCR (RT-PCR), or ligase chain reaction (LCR) using a nucleic acid isolated from the biological sample as a template in the PCR, RT-PCR, or LCR.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fees.

FIG. 1A-M. Samples. Provides a list of the human tissue samples used in the study of ERBB3 in human cancers.

FIG. 2A-B. Representative wild-type ERBB3 nucleic acid sequence (Accession No. NM_{—}001982) (SEQ ID NO: 1).

FIG. 3. Representative wild-type ERBB3 amino acid sequence (Accession No. NP_{—}001973) (SEQ ID NO: 2).

FIG. 4 (a-f). ERBB3 somatic mutations. (a-b) Protein alterations resulting from ERBB3 somatic mutations mapped over the ERBB3 protein domains are shown. Hotspot mutations depicted as repeating amino acid changes in a light red background. Height of the background vertical bar around the mutated residue is proportional to the frequency of mutation at that particular position. (c-d) ERBB3 non-synonymous somatic mutations (inverted triangles; red triangles depict hotspots) depicted over ERBB3 protein domains. The histogram on the top represents count of mutations at each position detected observed in samples in this study and other published studies (red bars indicate hot spot mutations and blue bars represent additional non-hotspot mutants tested for activity). (e-f) Expanded and supplemented view of FIG. 4 (a-b). FIG. 4 (a-f) provides a linear view of ErbB3 where FIG. 4a, c, and e show an N-terminal half, and FIG. 4b, d, and f show an C-terminal half.

FIG. 5A-B. Expression of ERBB3 mutants (A,B) and expression of ERBB2 (B) in the ERBB3 mutant colon samples as assessed using RNA-seq data (Seshagiri, S. et al. Comprehensive analysis of colon cancer genomes identifies recurrent mutations and R-spondin fusions. (Mansuscript in Preparation 2011)).

FIG. 6. Multiple sequence alignment ERBB3 ortholgos depicting conservation across mutated sites. H. sapiens (NP_{—}001973.2 (Full length sequence is disclosed as SEQ ID NO: 126 and the various regions are disclosed as SEQ ID NOS 132-151, respectively, in order of appearance)), P. troglodytes (XP_{—}509131.2 (Full length sequence is disclosed as SEQ ID NO: 130 and the various regions are disclosed as SEQ ID NOS 212-229, respectively, in order of appearance)), C. lupus (XP_{—}538226.2 (SEQ ID NO: 131)), B. taurus (NP_{—}001096575.1 (Full length sequence is disclosed as SEQ ID NO: 129 and the various regions are disclosed as SEQ ID NOS 192-211, respectively, in order of appearance)), M. musculus (NP 034283.1 (Full length sequence is disclosed as SEQ ID NO: 127 and the various regions are disclosed as SEQ ID NOS 152-171, respectively, in order of appearance)) and R. norvegicus (NP_{—}058914.2 (Full length sequence is disclosed as SEQ ID NO: 128 and the various regions are disclosed as SEQ ID NOS 172-191, respectively, in order of appearance)) were aligned using Clustal W (Larkin, M. A. et al. Bioinformatics (Oxford, England) 23, 2947-2948 (2007)). Mutated residues are show in a red oval background.

FIG. 7A-C. Frequent (or hotspot) somatic ECD mutations, shown in red, mapped on to (A) a crystal structure of “tethered” ERBB3 ECD [pdb 1M6B] (B), or (B) on to a model of “untethered” ERBB3/ERBB2 ECD heterodimer based on EGFR ECD dimer (pdb 1IVO), using ERBB3 [pdb 1M6B] and ERBB2 [pdb 1N8Z]. The ERBB3 ligand shown as a grey surface, based on EGF [pdb 1IVO] (C). ERBB3 kinase domain somatic mutations shown in red mapped on to a structure of the ERBB3 kinase domain [pdb 3LMG]. *=stop codon.

FIG. 8. ERBB3 somatic mutations mapped on to the ECD crystal structure of ERBB3 (pdb 1M6B) colored by domain.

FIG. 9. ERBB3 mutants support EGF-independent proliferation of MCF10A cells in 3D culture. MCF10A cells stably expressing ERBB3 mutants either alone or together with either EGFR or ERBB2 show EGF-independent proliferation. Studies involving MCF10A were performed in the absence of serum, EGF and NRG1. EV—empty vector.

FIG. 10a-c. ERBB3 mutants promote EGF and serum independent anchorage independent growth. Representative image depicting colonies formed by MCF10A expressing ERBB3 either alone or in combination with EGFR or ERBB2 are shown (a). Quantitation of the colonies from the assay depicted in (a) is shown for ERBB3-mutants in combination with EGFR (b) or ERBB2 (c).

FIG. 11A-C. MCF10A cells stably expressing ERBB3 mutants either alone (A) or together with either EGFR (B) or ERBB2 (C) show elevated downstream signaling as assessed by western blot. Studies involving MCF10A were performed in the absence of serum, EGF and NRG1. EV—empty vector.

FIG. 12A-C. ERBB3 mutants support EGF-independent proliferation of MCF10A cells in 3D culture. MCF10A cells stably expressing ERBB3 mutants either alone or together with either EGFR or ERBB2 show large acinar architecture, increased Ki67 staining and increased migration index compared to ERBB3/ERBB2 expressing MCF10A cells. Data represents mean±SEM of the three independent experiments. Studies involving MCF10A were performed in the absence of serum, EGF and NRG1. EV—empty vector.

FIG. 13A (a-b) shows representative images of MCF10A cells expressing the indicated ERBB3 mutants along with ERBB2 following migration from a transwell in the migration assay (a), and quantitation of this migration effect (b).

FIG. 13B (a-e) shows that ERBB3 mutants support anchorage independent growth of IMCE colonic epithelial cells. IMCE colonic epithelial cells expressing either ERBB3 by itself or in combination with ERBB2 showed anchorage independent growth (a), increased number of colonies (b), elevated phospho signaling (c, d) and in vivo growth (e) compared to ERBB3-WT/ERBB2 expressing IMCE cells. EV—empty vector.

FIG. 14. ERBB3 mutants transform and promote IL3-independent survival of BaF3 cells. BaF3 cells stably expressing ERBB3 mutants either alone or together with either EGFR or ERBB2 promotes IL3-independent survival. BaF3 studies were performed in the absence of IL-3 and NRG1. EV=empty vector; M=monomer & D=dimer.

FIG. 15A-C. ERBB3 mutants transform and promote IL3-independent survival of BaF3 cells. BaF3 cells stably expressing ERBB3 mutants either alone (A) or together with either EGFR (B) or ERBB2 (C) promotes an increase in phosphorylation of ERBB3 and its downstream effectors. BaF3 studies were performed in the absence of IL-3 and NRG1. EV=empty vector; M=monomer & D=dimer.

FIG. 16. A representative image of anchorage-independent growth of BaF3 cells stably expressing ERBB3 mutants either alone or in combination with either EGFR or ERBB2. BaF3 studies were performed in the absence of IL-3 and NRG1. EV=empty vector; M=monomer & D=dimer

FIG. 17. Anti-NRG1, a NRG1 neutralizing antibody, does not affect IL-3-independent survival of BaF3 cells promoted by ERBB3 mutants co-expressed with ERBB2. BaF3 studies were performed in the absence of IL-3 and NRG1. EV=empty vector; M=monomer & D=dimer

FIG. 18. Elevated levels of ERBB3 mutant/ERBB2 heterodimers in BaF3 cells in the absence of NRG1 as observed in immunoprecipitated material derived following cross linking the cell surface proteins using BS3. BaF3 studies were performed in the absence of IL-3 and NRG1. EV=empty vector; M=monomer & D=dimer.

FIG. 19. Elevated levels of ERBB3 mutant/ERBB2 heterodimers in BaF3 cells in the absence of NRG1 as observed on the cell surface detected using a proximity ligation assay 40. BaF3 studies were performed in the absence of IL-3 and NRG1. EV=empty vector; M=monomer & D=dimer

FIG. 20A-C. Quantitation of ERBB3-ERBB2 heterodimers. Images from Proximity ligation assay (FIG. 17) were analyzed using Duolink image software tool (Uppsala, Sweden). At least 100 cells from 5 to 6 image fields for the indicated combination of ERBB3 and ERBB2 expressing cells were analyzed for signal (red dots) resulting from ERBB2/ERBB3 dimers. The assay was performed with FLAG (ERBB3) and gD (ERBB2) antibody (A) or native ERBB3 and ERBB3 antibodies (B). Data are show as Mean±SEM. FIG. 20C shows that NRG1 was unable to support survival of BaF3 cells expressing ERBB3-WT or mutants alone.

FIG. 21. ERBB3 ECD mutants show increased IL-3 independent BaF3 survival in response to different dose of exongenous ligand NRG1. BaF3 studies were performed in the absence of IL-3. EV=empty vector; M=monomer & D=dimer

FIG. 22. ERBB3 mutants promote oncogenesis and lead to reduced overall survival. Kaplan-Meier survival curves for cohorts of mice implanted with BaF3 cells expressing indicated ERBB3 mutant/ERBB2 combination show reduced overall survival compared to control BaF3 (vector) cells (n=10 for arms; Log-rank test p<0.0001).

FIG. 23A-B. Flow cytometric analysis of total bone marrow cells (A) and spleen cells (B) isolated from mice receiving GFP-tagged BaF3 cells expressing the various ERBB3 mutants/ERBB2-WT.

FIG. 24A-B. Mean number of GFP positive cells in the bone marrow (A) and spleen (B) of mice (n=3) of the indicated study arms are shown.

FIG. 25A-B. Mean weight of spleen (A) and liver (B) from the mice (n=3) in the indicated study arms are depicted.

FIG. 26. Representative H&E-stained bone marrow (top), spleen (middle) and liver (bottom) sections from the same mice analyzed in FIG. 21. The bone marrow from empty vector animals consists of normal hematopoietic cells. *=infiltrating tumor cells, R=red pulp, W=lymphoid follicles of white pulp. In unmarked spleen section, there is a loss of red/white pulp architecture due to disruption by infiltrating tumor cells. The scale bar corresponds to 100 μm.

FIG. 27. Representative images of spleen and liver from mice transplanted with ERBB3 mutant expressing BaF3 cells are shown.

FIG. 28. Efficacy of anti-ERBB antibodies and small molecule inhibitors on oncogenic activity of ERBB3 mutants. Effect of targeted therapeutics on IL-3 independent proliferation of BaF3 cells stably expressing ERBB3 mutants together with ERBB2 as indicated in the figure.

FIG. 29. Representative images of the effect of targeted therapeutics on anchorage-independent growth of BaF3 cells stably expressing ERBB3 mutants together with ERBB2 as indicated in the figure.

FIG. 30. Schematic depicting the ERBB receptors and various targeted agents that were tested in this study.

FIG. 31A-B. Anti-ERBB3 antibodies are effectively targeting ERBB3 mutants in vivo. Efficacy of 10 mg/kg QW trastuzumab (Tmab), 50 mg/kg QW anti-ERBB3.1 and 100 mg/kg QW anti-ERBB3.2 antibodies in blocking leukemia-like disease induced by BaF3 cells expressing ERBB3 mutant G284R (A) or Q809R (B) in combination with ERBB2. Control antibody-treated group (Control Ab) receive 40 mg/kg QW anti-Ragweed antibody.

FIG. 32. Effect of targeted therapeutics on BaF3 cells stably expressing ERBB3 mutants together with ERBB2 as indicated in the figure. Concentration of antibodies and small molecule inhibitors used for treatment is same as indicated in FIG. 27.

FIG. 33. Effect of ERBB antibodies and small molecule inhibitors on phosphorylation of ERBB3 and downstream signaling molecules in BaF3 at 8 h after treatment is shown. Effect of these same agents at 24 h is shown in FIG. 30.

FIG. 34A-B. Proportion of infiltrating BaF3 cells expressing mutant ERBB3, G284R (A) and Q809R (B), in bone marrow (BM) and spleen following treatment with the antibodies as indicated in the figure.

FIG. 35A-B. Liver and spleen weight from animal implanted with ERBB3 mutant cells, G284R (A) and Q809R (B), following treatment with the antibodies as indicated.

FIG. 36. Infiltrating GFP positive BaF3 cell expressing ERBB3 mutant isolated from spleen and bone marrow of mice implanted with these cells are shown.

FIG. 37A-H. ERBB3 mutants transform and promote IL3-independent survival of BaF3 cells. (A) IL3-independent survival of BaF3 cells stably expressing ERBB3 mutants either alone or together with ERBB2 or ERBB2-KD. (B) A representative image of anchorage-independent growth of BaF3 cells stably expressing ERBB3 mutants either alone or in combination with either ERBB2 or ERBB2-KD. (C) Bar graph showing the number of colonies formed by BaF3 cells expressing the ERBB3 mutants along with ERBB2 show in (B). Very few colonies were formed by cells expressing ERBB3 mutants alone or in combination with ERBB2-KD. (D-F) Western blot showing pERBB3, pERBB2, pAKT and pERK status of BaF3 cells expressing ERBB3 mutants either alone (D) or in combination with ERBB2 (E) or ERBB2-KD (F). (G) Anti-NRG1, a NRG1 neutralizing antibody, does not affect IL-3-independent survival of BaF3 cells promoted by ERBB3 mutants co-expressed with ERBB2. (H) ERBB3 ECD mutants show increased IL-3 independent BaF3 survival in response to increasing dose of exogenous NRG1. BaF3 studies were performed in the absence of IL-3 (A-H) and NRG1 (A-F). EV=empty vector; M=monomer & D=dimer.

FIG. 38A-J. shRNA-mediated ERBB3 knockdown delays tumor growth. (A-J) CW-2 and DV-90 stably expressing inducible ERBB3 targeting shRNA upon dox-induction showed lower levels of ERBB3 and pERK (A, B), anchorage independent growth (C—F) and reduced in vivo growth (H, J) compared to uninduced cells (A-F) or cells expressing luciferase targeting shRNA (A-F, G & I). Data in (E, F) represent the number of anchorage independent colonies formed quantitated from multiple filed of images like the one show in (C, D). Data are shown as Mean±SEM.

FIG. 39A-C provides a nucleic acid sequence (SEQ ID NO: 3) and amino acid sequence (SEQ ID NO: 2) for ErbB3. The mutations of the present invention are indicated by the boxed amino acids and boxed/underlined codons.

### DETAILED DESCRIPTION OF THE INVENTION

The practice of the present invention will employ, unless otherwise indicated, conventional techniques of molecular biology (including recombinant techniques), microbiology, cell biology, and biochemistry, which are within the skill of the art. Such techniques are explained fully in the literature, such as, “Molecular Cloning: A Laboratory Manual”, 2^{nd} edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology”, 4^{th} edition (D. M. Weir & C. C. Blackwell, eds., Blackwell Science Inc., 1987); “Gene Transfer Vectors for Mammalian Cells” (J. M. Miller & M. P. Calos, eds., 1987); “Current Protocols in Molecular Biology” (F. M. Ausubel et al., eds., 1987); and “PCR: The Polymerase Chain Reaction”, (Mullis et al., eds., 1994).

### DEFINITIONS

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, the widely utilized molecular cloning methodologies described in Sambrook et al., Molecular Cloning: A Laboratory Manual 2nd. edition (1989) Cold Spring Harbor Laboratory Press, Cold Spring Harbor, N.Y. As appropriate, procedures involving the use of commercially available kits and reagents are generally carried out in accordance with manufacturer defined protocols and/or parameters unless otherwise noted. Before the present methods, kits and uses therefore are described, it is to be understood that this invention is not limited to the particular methodology, protocols, cell lines, animal species or genera, constructs, and reagents described as such may, of course, vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “and”, and “the” include plural referents unless the context clearly dictates otherwise.

Throughout this specification and claims, the word “comprise,” or variations such as “comprises” or “comprising,” will be understood to imply the inclusion of a stated integer or group of integers but not the exclusion of any other integer or group of integers.

The term “polynucleotide” or “nucleic acid,” as used interchangeably herein, refers to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping groups moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-2′-O-allyl, 2′-fluoro- or 2′-azido-ribose, carbocyclic sugar analogs, .alpha.-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), “(O)NR 2 (“amidate”), P(O)R, P(O)OR′, CO or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

“Oligonucleotide,” as used herein, refers to short, single stranded polynucleotides that are at least about seven nucleotides in length and less than about 250 nucleotides in length. Oligonucleotides may be synthetic. The terms “oligonucleotide” and “polynucleotide” are notmutually exclusive. The description above for polynucleotides is equally and fully applicable to oligonucleotides.

The term “primer” refers to a single stranded polynucleotide that is capable of hybridizing to a nucleic acid and allowing the polymerization of a complementary nucleic acid, generally by providing a free 3′—OH group.

As used herein, the term “gene” refers to a DNA sequence that encodes through its template or messenger RNA a sequence of amino acids characteristic of a specific peptide, polypeptide, or protein. The term “gene” also refers to a DNA sequence that encodes an RNA product. The term gene as used herein with reference to genomic DNA includes intervening, non-coding regions as well as regulatory regions and can include 5′ and 3′ ends.

The term “somatic mutation” or “somatic variation” refers to a change in a nucleotide sequence (e.g., an insertion, deletion, inversion, or substitution of one or more nucleotides), which is acquired in a cell of the body as opposed to a germ line cell. The term also encompasses the corresponding change in the complement of the nucleotide sequence, unless otherwise indicated.

The term “amino acid variation” refers to a change in an amino acid sequence (e.g., an insertion, substitution, or deletion of one or more amino acids, such as an internal deletion or an N- or C-terminal truncation) relative to a reference sequence.

The term “variation” refers to either a nucleotide variation or an amino acid variation.

The term “a genetic variation at a nucleotide position corresponding to a somatic mutation,” “a nucleotide variation at a nucleotide position corresponding to a somatic mutation,” and grammatical variants thereof refer to a nucleotide variation in a polynucleotide sequence at the relative corresponding DNA position occupied by said somatic mutation. The term also encompasses the corresponding variation in the complement of the nucleotide sequence, unless otherwise indicated.

The term “array” or “microarray” refers to an ordered arrangement of hybridizable array elements, preferably polynucleotide probes (e.g., oligonucleotides), on a substrate. The substrate can be a solid substrate, such as a glass slide, or a semi-solid substrate, such as nitrocellulose membrane.

The term “amplification” refers to the process of producing one or more copies of a reference nucleic acid sequence or its complement. Amplification may be linear or exponential (e.g., the polymerase chain reaction (PCR)). A “copy” does not necessarily mean perfect sequence complementarity or identity relative to the template sequence. For example, copies can include nucleotide analogs such as deoxyinosine, intentional sequence alterations (such as sequence alterations introduced through a primer comprising a sequence that is hybridizable, but not fully complementary, to the template), and/or sequence errors that occur during amplification.

The term “mutation-specific oligonucleotide” refers to an oligonucleotide that hybridizes to a region of a target nucleic acid that comprises a nucleotide variation (often a substitution). “Somatic mutation-specific hybridization” means that, when a mutation-specific oligonucleotide is hybridized to its target nucleic acid, a nucleotide in the mutation-specific oligonucleotide specifically base pairs with the nucleotide variation. An somatic mutation-specific oligonucleotide capable of mutation-specific hybridization with respect to a particular nucleotide variation is said to be “specific for” that variation.

The term “mutation-specific primer” refers to an mutation-specific oligonucleotide that is a primer.

The term “primer extension assay” refers to an assay in which nucleotides are added to a nucleic acid, resulting in a longer nucleic acid, or “extension product,” that is detected directly or indirectly. The nucleotides can be added to extend the 5′ or 3′ end of the nucleic acid.

The term “mutation-specific nucleotide incorporation assay” refers to a primer extension assay in which a primer is (a) hybridized to target nucleic acid at a region that is 3′ or 5′ of a nucleotide variation and (b) extended by a polymerase, thereby incorporating into the extension product a nucleotide that is complementary to the nucleotide variation.

The term “mutation-specific primer extension assay” refers to a primer extension assay in which a mutation-specific primer is hybridized to a target nucleic acid and extended.

The term “mutation-specific oligonucleotide hybridization assay” refers to an assay in which (a) a mutation-specific oligonucleotide is hybridized to a target nucleic acid and (b) hybridization is detected directly or indirectly.

The term “5′ nuclease assay” refers to an assay in which hybridization of a mutation-specific oligonucleotide to a target nucleic acid allows for nucleolytic cleavage of the hybridized probe, resulting in a detectable signal.

The term “assay employing molecular beacons” refers to an assay in which hybridization of a mutation-specific oligonucleotide to a target nucleic acid results in a level of detectable signal that is higher than the level of detectable signal emitted by the free oligonucleotide.

The term “oligonucleotide ligation assay” refers to an assay in which a mutation-specific oligonucleotide and a second oligonucleotide are hybridized adjacent to one another on a target nucleic acid and ligated together (either directly or indirectly through intervening nucleotides), and the ligation product is detected directly or indirectly.

The term “target sequence,” “target nucleic acid,” or “target nucleic acid sequence” refers generally to a polynucleotide sequence of interest in which a nucleotide variation is suspected or known to reside, including copies of such target nucleic acid generated by amplification.

The term “detection” includes any means of detecting, including direct and indirect detection.

The terms “cancer” and “cancerous” refer to or describe the physiological condition in mammals that is typically characterized by unregulated cell growth. The cancer diagnosed in accordance with the present invention is any type of cancer characterized by the presence of an ErbB3 mutation, specifically including metastatic or locally advanced non-resectable cancer, including, without limitation, gastric, colon, esophageal, rectal, cecum, colorectal, non-small-cell lung (NSCLC) adenocarinoma, NSCLC (Squamous carcinoma), renal carcinoma, melanoma, ovarian, lung large cell, small-cell lung cancer (SCLC), hepatocellular (HCC), lung cancer, head & neck cancer, and pancreatic cancer.

As used herein, a subject “at risk” of developing cancer may or may not have detectable disease or symptoms of disease, and may or may not have displayed detectable disease or symptoms of disease prior to the diagnostic methods described herein. “At risk” denotes that a subject has one or more risk factors, which are measurable parameters that correlate with development of cancer, as described herein and known in the art. A subject having one or more of these risk factors has a higher probability of developing cancer than a subject without one or more of these risk factor(s).

The term “diagnosis” is used herein to refer to the identification or classification of a molecular or pathological state, disease or condition, for example, cancer. “Diagnosis” may also refer to the classification of a particular sub-type of cancer, e.g., by molecular features (e.g., a patient subpopulation characterized by nucleotide variation(s) in a particular gene or nucleic acid region).

The term “aiding diagnosis” is used herein to refer to methods that assist in making a clinical determination regarding the presence, or nature, of a particular type of symptom or condition of cancer. For example, a method of aiding diagnosis of cancer can comprise measuring the presence of absence of one or more genetic markers indicative of cancer or an increased risk of having cancer in a biological sample from an individual.

The term “prognosis” is used herein to refer to the prediction of the likelihood of developing cancer. The term “prediction” is used herein to refer to the likelihood that a patient will respond either favorably or unfavorably to a drug or set of drugs. In one embodiment, the prediction relates to the extent of those responses. In one embodiment, the prediction relates to whether and/or the probability that a patient will survive or improve following treatment, for example treatment with a particular therapeutic agent, and for a certain period of time without disease recurrence. The predictive methods of the invention can be used clinically to make treatment decisions by choosing the most appropriate treatment modalities for any particular patient. The predictive methods of the present invention are valuable tools in predicting if a patient is likely to respond favorably to a treatment regimen, such as a given therapeutic regimen, including for example, administration of a given therapeutic agent or combination, surgical intervention, steroid treatment, etc., or whether long-term survival of the patient, following a therapeutic regimen is likely.

As used herein, “treatment” refers to clinical intervention in an attempt to alter the natural course of the individual or cell being treated, and can be performed before or during the course of clinical pathology. Desirable effects of treatment include preventing the occurrence or recurrence of a disease or a condition or symptom thereof, alleviating a condition or symptom of the disease, diminishing any direct or indirect pathological consequences of the disease, decreasing the rate of disease progression, ameliorating or palliating the disease state, and achieving remission or improved prognosis. In some embodiments, methods and compositions of the invention are useful in attempts to delay development of a disease or disorder.

An “cancer therapeutic agent”, a “therapeutic agent effective to treat cancer”, and grammatical variations thereof, as used herein, refer to an agent that when provided in an effective amount is known, clinically shown, or expected by clinicians to provide a therapeutic benefit in a subject who has cancer. In one embodiment, the phrase includes any agent that is marketed by a manufacturer, or otherwise used by licensed clinicians, as a clinically-accepted agent that when provided in an effective amount would be expected to provide a therapeutic effect in a subject who has cancer. In various non-limiting embodiments, a cancer therapeutic agent comprises chemotherapy agents, HER dimerization inhibitors, HER antibodies, antibodies directed against tumor associated antigens, anti-hormonal compounds, cytokines, EGFR-targeted drugs, anti-angiogenic agents, tyrosine kinase inhibitors, growth inhibitory agents and antibodies, cytotoxic agents, antibodies that induce apoptosis, COX inhibitors, farnesyl transferase inhibitors, antibodies that binds oncofetal protein CA 125, HER2 vaccines, Raf or ras inhibitors, liposomal doxorubicin, topotecan, taxene, dual tyrosine kinase inhibitors, TLK286, EMD-7200, pertuzumab, trastuzumab, erlotinib, and bevacizumab.

A “chemotherapy” is use of a chemical compound useful in the treatment of cancer. Examples of chemotherapeutic agents, used in chemotherapy, include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; TLK 286 (TELCYTA™); acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; bisphosphonates, such as clodronate; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)) and anthracyclines such as annamycin, AD 32, alcarubicin, daunorubicin, dexrazoxane, DX-52-1, epirubicin, GPX-100, idarubicin, KRN5500, menogaril, dynemicin, including dynemicin A, an esperamicin, neocarzinostatin chromophore and related chromoprotein enediyne antiobiotic chromophores, aclacinomysins, actinomycin, authramycin, azaserine, bleomycins, cactinomycin, carabicin, caminomycin, carzinophilin, chromomycinis, dactinomycin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin, liposomal doxorubicin, and deoxydoxorubicin), esorubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; folic acid analogues such as denopterin, pteropterin, and trimetrexate; purine analogs such as fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, and floxuridine; androgens such as calusterone, dromostanolone propionate, epitiostanol, mepitiostane, and testolactone; anti-adrenals such as aminoglutethimide, mitotane, and trilostane; folic acid replenisher such as folinic acid (leucovorin); aceglatone; anti-folate anti-neoplastic agents such as ALIMTA®, LY231514 pemetrexed, dihydrofolate reductase inhibitors such as methotrexate, anti-metabolites such as 5-fluorouracil (5-FU) and its prodrugs such as UFT, S-1 and capecitabine, and thymidylate synthase inhibitors and glycinamide ribonucleotide formyltransferase inhibitors such as raltitrexed (TOMUDEX®, TDX); inhibitors of dihydropyrimidine dehydrogenase such as eniluracil; aldophosphamide glycoside; aminolevulinic acid; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK7 polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE™, FILDESIN®); dacarbazine; mannomustine; mitobronitol; mitolactol; pipobroman; gacytosine; arabinoside (“Ara-C”); cyclophosphamide; thiotepa; taxoids and taxenes, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® docetaxel (Rhone-Poulenc Rorer, Antony, France); chloranbucil; gemcitabine (GEMZAR®); 6-thioguanine; mercaptopurine; platinum; platinum analogs or platinum-based analogs such as cisplatin, oxaliplatin and carboplatin; vinblastine (VELBAN®); etoposide (VP-16); ifosfamide; mitoxantrone; vincristine (ONCOVIN®); vinca alkaloid; vinorelbine (NAVELBINE®); novantrone; edatrexate; daunomycin; aminopterin; xeloda; ibandronate; topoisomerase inhibitor RFS 2000; difluoromethylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovorin.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

An “effective amount” refers to an amount effective, at dosages and for periods of time necessary, to achieve the desired therapeutic or prophylactic result. A “therapeutically effective amount” of a therapeutic agent may vary according to factors such as the disease state, age, sex, and weight of the individual, and the ability of the antibody to elicit a desired response in the individual. A therapeutically effective amount is also one in which any toxic or detrimental effects of the therapeutic agent are outweighed by the therapeutically beneficial effects. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

An “individual,” “subject” or “patient” is a vertebrate. In certain embodiments, the vertebrate is a mammal. Mammals include, but are not limited to, primates (including human and non-human primates) and rodents (e.g., mice and rats). In certain embodiments, a mammal is a human.

A “patient subpopulation,” and grammatical variations thereof, as used herein, refers to a patient subset characterized as having one or more distinctive measurable and/or identifiable characteristics that distinguishes the patient subset from others in the broader disease category to which it belongs. Such characteristics include disease subcategories, gender, lifestyle, health history, organs/tissues involved, treatment history, etc. In one embodiment, a patient subpopulation is characterized by nucleic acid signatures, including nucleotide variations in particular nucleotide positions and/or regions (such as somatic mutations).

A “control subject” refers to a healthy subject who has not been diagnosed as having cancer and who does not suffer from any sign or symptom associated with cancer.

The term “sample”, as used herein, refers to a composition that is obtained or derived from a subject of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized.

By “tissue or cell sample” is meant a collection of similar cells obtained from a tissue of a subject or patient. The source of the tissue or cell sample may be solid tissue as from a fresh, frozen and/or preserved organ or tissue sample or biopsy or aspirate; blood or any blood constituents; bodily fluids such as serum, urine, sputum, or saliva. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like. A “reference sample”, “reference cell”, “reference tissue”, “control sample”, “control cell”, or “control tissue”, as used herein, refers to a sample, cell or tissue obtained from a source known, or believed, not to be afflicted with the disease or condition for which a method or composition of the invention is being used to identify. In one embodiment, a reference sample, reference cell, reference tissue, control sample, control cell, or control tissue is obtained from a healthy part of the body of the same subject or patient in whom a disease or condition is being identified using a composition or method of the invention. In one embodiment, a reference sample, reference cell, reference tissue, control sample, control cell, or control tissue is obtained from a healthy part of the body of an individual who is not the subject or patient in whom a disease or condition is being identified using a composition or method of the invention.

For the purposes herein a “section” of a tissue sample is meant a single part or piece of a tissue sample, e.g. a thin slice of tissue or cells cut from a tissue sample. It is understood that multiple sections of tissue samples may be taken and subjected to analysis according to the present invention, provided that it is understood that the present invention comprises a method whereby the same section of tissue sample is analyzed at both morphological and molecular levels, or is analyzed with respect to both protein and nucleic acid.

By “correlate” or “correlating” is meant comparing, in any way, the performance and/or results of a first analysis or protocol with the performance and/or results of a second analysis or protocol. For example, one may use the results of a first analysis or protocol in carrying out a second protocol and/or one may use the results of a first analysis or protocol to determine whether a second analysis or protocol should be performed. With respect to the embodiment of gene expression analysis or protocol, one may use the results of the gene expression analysis or protocol to determine whether a specific therapeutic regimen should be performed.

A “small molecule” or “small organic molecule” is defined herein as an organic molecule having a molecular weight below about 500 Daltons.

The word “label” when used herein refers to a detectable compound or composition. The label may be detectable by itself (e.g., radioisotope labels or fluorescent labels) or, in the case of an enzymatic label, may catalyze chemical alteration of a substrate compound or composition which results in a detectable product. Radionuclides that can serve as detectable labels include, for example, I-131, I-123, I-125, Y-90, Re-188, Re-186, At −211, Cu-67, Bi-212, and Pd-109.

Reference to “about” a value or parameter herein includes (and describes) embodiments that are directed to that value or parameter per se. For example, description referring to “about X” includes description of “X.”

The term “package insert” is used to refer to instructions customarily included in commercial packages of therapeutic products, that contain information about the indications, usage, dosage, administration, combination therapy, contraindications and/or warnings concerning the use of such therapeutic products.

The terms “antibody” and “immunoglobulin” are used interchangeably in the broadest sense and include monoclonal antibodies (e.g., full length or intact monoclonal antibodies), polyclonal antibodies, monovalent antibodies, multivalent antibodies, multispecific antibodies (e.g., bispecific antibodies so long as they exhibit the desired biological activity) and may also include certain antibody fragments (as described in greater detail herein). An antibody can be chimeric, human, humanized and/or affinity matured. “Antibody fragments” comprise a portion of an intact antibody, preferably comprising the antigen binding region thereof. Examples of antibody fragments include Fab, Fab′, F(ab′)₂, and Fv fragments; diabodies; linear antibodies; single-chain antibody molecules; and multispecific antibodies formed from antibody fragments.

An antibody of this invention “which binds” an antigen of interest is one that binds the antigen with sufficient affinity such that the antibody is useful as a diagnostic and/or therapeutic agent in targeting a protein or a cell or tissue expressing the antigen. With regard to the binding of a antibody to a target molecule, the term “specific binding” or “specifically binds to” or is “specific for” a particular polypeptide or an epitope on a particular polypeptide target means binding that is measurably different from a non-specific interaction. Specific binding can be measured, for example, by determining binding of a molecule compared to binding of a control molecule. For example, specific binding can be determined by competition with a control molecule that is similar to the target, for example, an excess of non-labeled target. In this case, specific binding is indicated if the binding of the labeled target to a probe is competitively inhibited by excess non-labeled target. In one particular embodiment, “specifically binds” refers to binding of an antibody to its specified target HER receptors and not other specified non-target HER receptors. For example, an anti-HER3 antibody specifically binds to HER3 but does not specifically bind to EGFR, HER2, or HER4. An EGFR/HER3 bispecific antibody specifically binds to EGFR and HER3 but does not specifically bind to HER2 or HER4.

A “HER receptor” or “ErbB receptor” is a receptor protein tyrosine kinase which belongs to the HER receptor family and includes EGFR (ErbB1, HER1), HER2 (ErbB2), HER3 (ErbB3) and HER4 (ErbB4) receptors. The HER receptor will generally comprise an extracellular domain, which may bind an HER ligand and/or dimerize with another HER receptor molecule; a lipophilic transmembrane domain; a conserved intracellular tyrosine kinase domain; and a carboxyl-terminal signaling domain harboring several tyrosine residues which can be phosphorylated. The HER receptor may be a “native sequence” HER receptor or an “amino acid sequence variant” thereof. Preferably the HER receptor is a native sequence human HER receptor. The “HER pathway” refers to the signaling network mediated by the HER receptor family.

The terms “ErbB1”, “HER1”, “epidermal growth factor receptor” and “EGFR” are used interchangeably herein and refer to EGFR as disclosed, for example, in Carpenter et al Ann. Rev. Biochem. 56:881-914 (1987), including naturally occurring mutant forms thereof (e.g. a deletion mutant EGFR as in Ullrich et al, Nature (1984) 309:418425 and Humphrey et al. PNAS (USA) 87:4207-4211 (1990)), as well we variants thereof, such as EGFRvIII. Variants of EGFR also include deletional, substitutional and insertional variants, for example those described in Lynch et al (New England Journal of Medicine 2004, 350:2129), Paez et al (Science 2004, 304:1497), and Pao et al (PNAS 2004, 101:13306). Herein, “EGFR extracellular domain” or “EGFR ECD” refers to a domain of EGFR that is outside of a cell, either anchored to a cell membrane, or in circulation, including fragments thereof. In one embodiment, the extracellular domain of EGFR may comprise four domains: “Domain I” (amino acid residues from about 1-158, “Domain II” (amino acid residues 159-336), “Domain III” (amino acid residues 337-470), and “Domain IV” (amino acid residues 471-645), where the boundaries are approximate, and may vary by about 1-3 amino acids.

The expressions “ErbB2” and “HER2” are used interchangeably herein and refer to human HER2 protein described, for example, in Semba et al, PNAS (USA) 82:6497-6501 (1985) and Yamamoto et al. Nature 319:230-234 (1986) (GenBank accession number X03363). The term “er£B2” refers to the gene encoding human HER2 and “neu” refers to the gene encoding rat pi 85″ea. Preferred HER2 is native sequence human HER2.

Herein, “HER2 extracellular domain” or “HER2ECD” refers to a domain of HER2 that is outside of a cell, either anchored to a cell membrane, or in circulation, including fragments thereof. In one embodiment, the extracellular domain of HER2 may comprise four domains: “Domain I” (amino acid residues from about 1-195, “Domain II” (amino acid residues from about 196-319), “Domain III” (amino acid residues from about 320-488), and “Domain IV” (amino acid residues from about 489-630) (residue numbering without signal peptide). See Garrett et al. MoI. Cell. 11: 495-505 (2003), Cho et al Nature All: 756-760 (2003), Franklin et al Cancer Cell 5:317-328 (2004), and Plowman et al Proc. Natl. Acad. ScL 90:1746-1750 (1993).

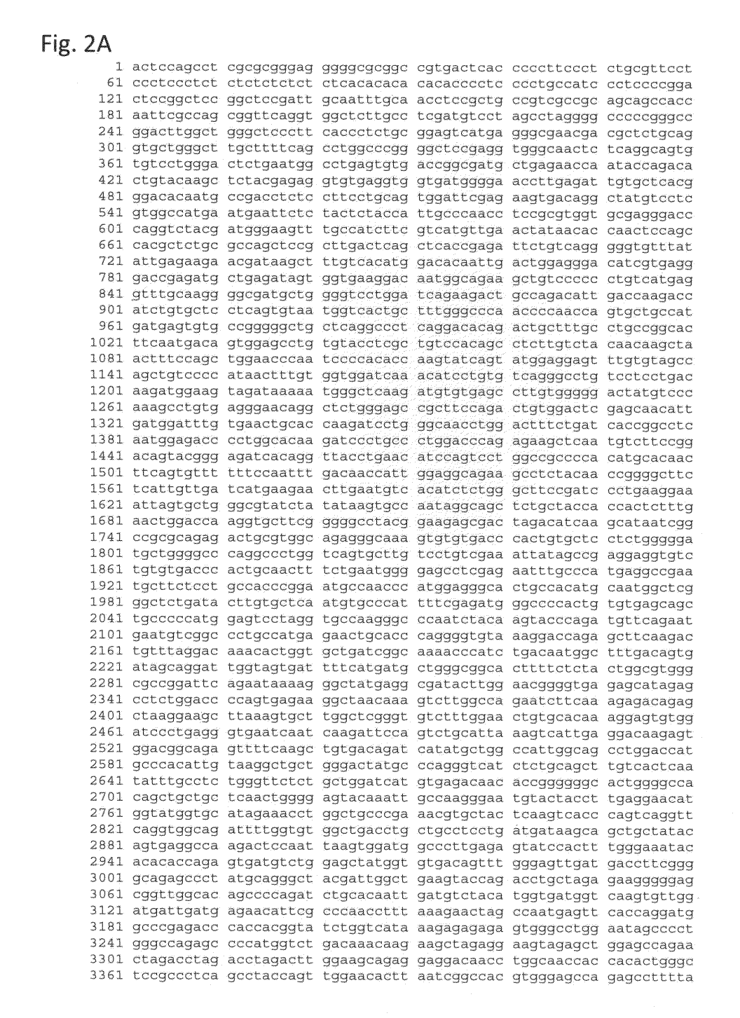

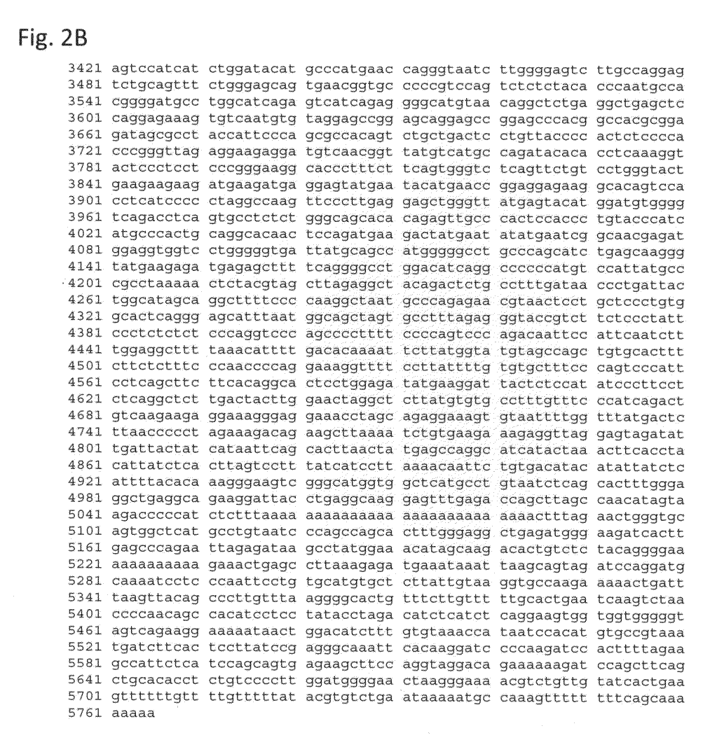

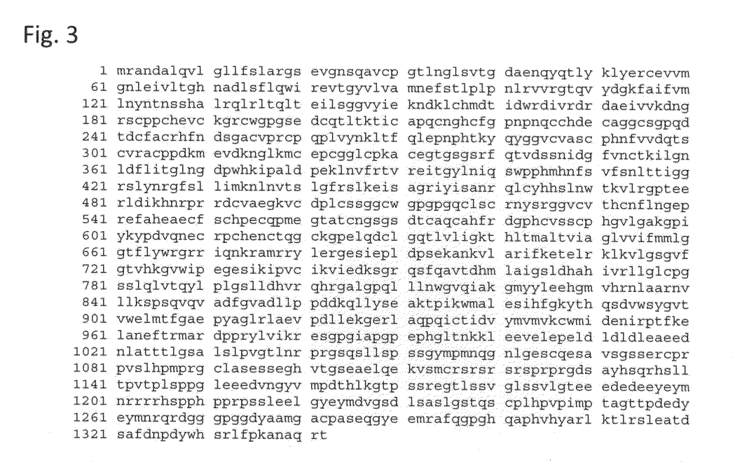

“ErbB3” and “HER3” refer to the receptor polypeptide as disclosed, for example, in U.S. Pat. Nos. 5,183,884 and 5,480,968 as well as Kraus et al. PNAS (USA) 86:9193-9197 (1989) (see also FIGS. 2 and 3)

Herein, “HER3 extracellular domain” or “HER3ECD” or “ErbB3 extracellular domain” refers to a domain of HER3 that is outside of a cell, either anchored to a cell membrane, or in circulation, including fragments thereof. In one embodiment, the extracellular domain of HER3 may comprise four domains: Domain I, Domain II, Domain III, and Domain IV. In one embodiment, the HER3ECD comprises amino acids 1-636 (numbering including signal peptide). In one embodiment, HER3 domain III comprises amino acids 328-532 (numbering including signal peptide.

The terms “ErbB4” and “HER4” herein refer to the receptor polypeptide as disclosed, for example, in EP Pat Appin No 599,274; Plowman et al, Proc. Natl. Acad. ScL USA, 90:1746-1750 (1993); and Plowman et al, Nature, 366:473-475 (1993), including isoforms thereof, e.g., as disclosed in WO99/19488, published Apr. 22, 1999. By “HER ligand” is meant a polypeptide which binds to and/or activates a HER receptor. The HER ligand of particular interest herein is a native sequence human HER ligand such as epidermal growth factor (EGF) (Savage et al, J. Biol. Chem. 247:7612-7621 (1972)); transforming growth factor alpha (TGF-α) (Marquardt et al, Science 223:1079-1082 (1984)); amphiregulin also known as schwanoma or keratinocyte autocrine growth factor (Shoyab et al Science 243:1074-1076 (1989); Kimura et al Nature 348:257-260 (1990); and Cook et al MoI Cell Biol. 11:2547-2557 (1991)); betacellulin (Shing et al, Science 259:1604-1607 (1993); and Sasada et al Biochem. Biophys. Res. Commun. 190:1173 (1993)); heparin-binding epidermal growth factor (HB-EGF) (Higashiyama et al, Science 251:936-939 (1991)); epiregulin (Toyoda et al, J. Biol. Chem. 270:7495-7500 (1995); and Komurasaki et al Oncogene 15:2841-2848 (1997)); a heregulin (see below); neuregulin-2 (NRG-2) (Carraway et al, Nature 387:512-516 (1997)); neuregulin-3 (NRG-3) (Zhang et al, Proc. Natl. Acad. ScL 94:9562-9567 (1997)); neuregulin-4 (NRG-4) (Harari et al Oncogene 18:2681-89 (1999)); and cripto (CR—I) (Kanmm et al. J. Biol. Chem. 272(6):3330-3335 (1997)). HER ligands which bind EGFR include EGF, TGF-α, amphiregulin, betacellulin, HB-EGF and epiregulin. HER ligands which bind HER3 include heregulins and NRG-2. HER ligands capable of binding HER4 include betacellulin, epiregulin, HB-EGF, NRG-2, NRG-3, NRG-4, and heregulins.

“Heregulin” (HRG) when used herein refers to a polypeptide encoded by the heregulin gene product as disclosed in U.S. Pat. No. 5,641,869, or Marchionni et al, Nature, 362:312-318 (1993). Examples of heregulins include heregulin-α, heregulin-β1, heregulin-β2 and heregulin-β3 (Holmes et al, Science, 256:1205-1210 (1992); and U.S. Pat. No. 5,641,869); neu differentiation factor (NDF) (Peles et al Cell 69: 205-216 (1992)); acetylcholine receptor-inducing activity (ARIA) (Falls et al. Cell 72:801-815 (1993)); glial growth factors (GGFs) (Marchionni et al., Nature, 362:312-318 (1993)); sensory and motor neuron derived factor (SMDF) (Ho et al. J. Biol. Chem. 270:14523-14532 (1995)); γ-heregulin (Schaefer et al. Oncogene 15:1385-1394 (1997)). A “HER dimer” herein is a noncovalently associated dimer comprising at least two HER receptors. Such complexes may form when a cell expressing two or more HER receptors is exposed to an HER ligand and can be isolated by immunoprecipitation and analyzed by SDS-PAGE as described in Sliwkowski et al, J. Biol. Chem., 269(20):14661-14665 (1994), for example. Other proteins, such as a cytokine receptor subunit (e.g. gp130) may be associated with the dimer

A “HER heterodimer” herein is a noncovalently associated heterodimer comprising at least two different HER receptors, such as EGFR-HER2, EGFR-HER3, EGFR-HER4, HER2-HER3 or HER2-HER4 heterodimers.

A “HER inhibitor” or “ErbB inhibitor” or “ErbB antagonist” is an agent which interferes with HER activation or function. Examples of HER inhibitors include HER antibodies (e.g. EGFR, HER2, HER3, or HER4 antibodies); EGFR-targeted drugs; small molecule HER antagonists; HER tyrosine kinase inhibitors; HER2 and EGFR dual tyrosine kinase inhibitors such as lapatinib/GW572016; antisense molecules (see, for example, WO2004/87207); and/or agents that bind to, or interfere with function of, downstream signaling molecules, such as MAPK or Akt. Preferably, the HER inhibitor is an antibody which binds to a HER receptor. In general, a HER inhibitor refers to those compounds that specifically bind to a particular HER receptor and prevent or reduce its signaling activity, but do not specifically bind to other HER receptors. For example, a HER3 antagonist specifically binds to reduce its activity, but does not specifically bind to EGFR, HER2, or HER4.

A “HER dimerization inhibitor” or “HDI” is an agent which inhibits formation of a HER homodimer or HER heterodimer. Preferably, the HER dimerization inhibitor is an antibody. However, HER dimerization inhibitors also include peptide and non-peptide small molecules, and other chemical entities which inhibit the formation of HER homo- or heterodimers.

An antibody which “inhibits HER dimerization” is an antibody which inhibits, or interferes with, formation of a HER dimer, regardless of the underlying mechanism. In one embodiment, such an antibody binds to HER2 at the heterodimeric binding site thereof. One particular example of a dimerization inhibiting antibody is pertuzumab (Pmab), or MAb 2C4. Other examples of HER dimerization inhibitors include antibodies which bind to EGFR and inhibit dimerization thereof with one or more other HER receptors (for example EGFR monoclonal antibody 806, MAb 806, which binds to activated or “untethered” EGFR; see Johns et al, J. Biol. Chem. 279(29):30375-30384 (2004)); antibodies which bind to HER3 and inhibit dimerization thereof with one or more other HER receptors; antibodies which bind to HER4 and inhibit dimerization thereof with one or more other HER receptors; peptide dimerization inhibitors (U.S. Pat. No. 6,417,168); antisense dimerization inhibitors; etc.

As used herein, “HER2 antagonist” or “EGFR inhibitor” refer to those compounds that specifically bind to EGFR and prevent or reduce its signaling activity, and do not specifically bind to HER2, HER3, or HER4. Examples of such agents include antibodies and small molecules that bind to EGFR. Examples of antibodies which bind to EGFR include

As used herein, “EGFR antagonist” or “EGFR inhibitor” refer to those compounds that specifically bind to EGFR and prevent or reduce its signaling activity, and do not specifically bind to HER2, HER3, or HER4. Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBITUX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6. 3 and E7.6. 3 and described in U.S. Pat. No. 6,235,883; MDX-447 (Medarex Inc); and mAb 806 or humanized mAb 806 (Johns et al, J. Biol. Chem. 279(29):30375-30384 (2004)). The anti-EGFR antibody may be conjugated with a cytotoxic agent, thus generating an immunoconjugate (see, e.g., EP659,439A2, Merck patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI-1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N-8-(3-chloro-4-fluoro-phenyl)-N-2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Sugen); and AG1571 (SU 5271; Sugen).

A “HER antibody” is an antibody that binds to a HER receptor. Optionally, the HER antibody further interferes with HER activation or function. Particular HER2 antibodies include pertuzumab and trastuzumab. Examples of particular EGFR antibodies include cetuximab and panitumumab. Patent publications related to HER antibodies include: U.S. Pat. No. 5,677,171, U.S. Pat. No. 5,720,937, U.S. Pat. No. 5,720,954, U.S. Pat. No. 5,725,856, U.S. Pat. No. 5,770,195, U.S. Pat. No. 5,772,997, U.S. Pat. No. 6,165,464, U.S. Pat. No. 6,387,371, U.S. Pat. No. 6,399,063, US2002/019221 IA1, U.S. Pat. No. 6,015,567, U.S. Pat. No. 6,333,169, U.S. Pat. No. 4,968,603, U.S. Pat. No. 5,821,337, U.S. Pat. No. 6,054,297, U.S. Pat. No. 6,407,213, U.S. Pat. No. 6,719,971, U.S. Pat. No. 6,800,738, US2004/0236078A1, U.S. Pat. No. 5,648,237, U.S. Pat. No. 6,267,958, U.S. Pat. No. 6,685,940, U.S. Pat. No. 6,821,515, WO98/17797, U.S. Pat. No. 6,333,398, U.S. Pat. No. 6,797,814, U.S. Pat. No. 6,339,142, U.S. Pat. No. 6,417,335, U.S. Pat. No. 6,489,447, WO99/31140, US2003/0147884A1, US2003/0170234A1, US2005/0002928A1, U.S. Pat. No. 6,573,043, US2003/0152987A1, WO99/48527, US2002/0141993A1, WO01/00245, US2003/0086924, US2004/0013667A1, WO00/69460, WO01/00238, WO01/15730, U.S. Pat. No. 6,627,196B1, U.S. Pat. No. 6,632,979B1, WO01/00244, US2002/0090662A1, WO01/89566, US2002/0064785, US2003/0134344, WO 04/24866, US2004/0082047, US2003/0175845A1, WO03/087131, US2003/0228663, WO2004/008099A2, US2004/0106161, WO2004/048525, US2004/0258685A1, U.S. Pat. No. 5,985,553, U.S. Pat. No. 5,747,261, U.S. Pat. No. 4,935,341, U.S. Pat. No. 5,401,638, U.S. Pat. No. 5,604,107, WO 87/07646, WO 89/10412, WO 91/05264, EP 412,116 B1, EP 494,135B1,U.S. Pat. No. 5,824,311, EP 444,181B1, EP 1,006,194 A2, US 2002/0155527A1, WO 91/02062, U.S. Pat. No. 5,571,894, U.S. Pat. No. 5,939,531, EP 502,812B1, WO 93/03741, EP 554,441 B1, EP 656,367 A1, U.S. Pat. No. 5,288,477, U.S. Pat. No. 5,514,554, U.S. Pat. No. 5,587,458, WO 93/12220, WO 93/16185, U.S. Pat. No. 5,877,305, WO 93/21319, WO 93/21232, U.S. Pat. No. 5,856,089, WO 94/22478, U.S. Pat. No. 5,910,486, U.S. Pat. No. 6,028,059, WO 96/07321, U.S. Pat. No. 5,804,396, U.S. Pat. No. 5,846,749, EP 711,565, WO 96/16673, U.S. Pat. No. 5,783,404, U.S. Pat. No. 5,977,322, U.S. Pat. No. 6,512,097, WO 97/00271, U.S. Pat. No. 6,270,765, U.S. Pat. No. 6,395,272, U.S. Pat. No. 5,837,243, WO 96/40789, U.S. Pat. No. 5,783,186, U.S. Pat. No. 6,458,356, WO 97/20858, WO 97/38731, U.S. Pat. No. 6,214,388, U.S. Pat. No. 5,925,519, WO 98/02463, U.S. Pat. No. 5,922,845, WO 98/18489, WO 98/33914, U.S. Pat. No. 5,994,071, WO 98/45479, U.S. Pat. No. 6,358,682 B1, US 2003/0059790, WO 99/55367, WO 01/20033, US 2002/0076695 A1, WO 00/78347, WO 01/09187, WO 01/21192, WO 01/32155, WO 01/53354, WO 01/56604, WO 01/76630, WO02/05791, WO 02/11677, U.S. Pat. No. 6,582,919, US2002/0192652A1, US 2003/0211530A1, WO 02/44413, US 2002/0142328, U.S. Pat. No. 6,602,670 B2, WO 02/45653, WO 02/055106, US2003/0152572, US 2003/0165840, WO 02/087619, WO 03/006509, WO03/012072, WO 03/028638, US 2003/0068318, WO 03/041736, EP 1,357,132, US 2003/0202973, US 2004/0138160, U.S. Pat. No. 5,705,157, U.S. Pat. No. 6,123,939, EP 616,812 B1, US 2003/0103973, US 2003/0108545, U.S. Pat. No. 6,403,630 B1, WO 00/61145, WO 00/61185, U.S. Pat. No. 6,333,348 B1, WO 01/05425, WO 01/64246, US 2003/0022918, US 2002/0051785 A1, U.S. Pat. No. 6,767,541, WO 01/76586, US 2003/0144252, WO 01/87336, US 2002/0031515 A1, WO 01/87334, WO 02/05791, WO 02/09754, US 2003/0157097, US 2002/0076408, WO 02/055106, WO 02/070008, WO 02/089842 WO 11/076683 and WO 03/86467.

“HER activation” refers to activation, or phosphorylation, of any one or more HER receptors. Generally, HER activation results in signal transduction (e.g. that caused by an intracellular kinase domain of a HER receptor phosphorylating tyrosine residues in the HER receptor or a substrate polypeptide). HER activation may be mediated by HER ligand binding to a HER dimer comprising the HER receptor of interest. HER ligand binding to a HER dimer may activate a kinase domain of one or more of the HER receptors in the dimer and thereby results in phosphorylation of tyrosine residues in one or more of the HER receptors and/or phosphorylation of tyrosine residues in additional substrate polypeptides(s), such as Akt or MAPK intracellular kinases.

“Phosphorylation” refers to the addition of one or more phosphate group(s) to a protein, such as a HER receptor, or substrate thereof.

A “heterodimeric binding site” on HER2, refers to a region in the extracellular domain of HER2 that contacts, or interfaces with, a region in the extracellular domain of EGFR, HER3 or HER4 upon formation of a dimer therewith. The region is found in Domain II of HER2. Franklin et al. Cancer Cell 5:317-328 (2004).

A HER2 antibody that “binds to a heterodimeric binding site” of HER2, binds to residues in domain II (and optionally also binds to residues in other of the domains of the HER2 extracellular domain, such as domains I and III), and can sterically hinder, at least to some extent, formation of a HER2-EGFR, HER2-HER3, or HER2-HER4 heterodimer. Franklin et al. Cancer Cell 5:317-328 (2004) characterize the HER2-pertuzumab crystal structure, deposited with the RCSB Protein Data Bank (ID Code IS78), illustrating an exemplary antibody that binds to the heterodimeric binding site of HER2. An antibody that “binds to domain II” of HER2 binds to residues in domain II and optionally residues in other domain(s) of HER2, such as domains I and III.

“Isolated,” when used to describe the various antibodies disclosed herein, means an antibody that has been identified and separated and/or recovered from a cell or cell culture from which it was expressed. Contaminant components of its natural environment are materials that would typically interfere with diagnostic or therapeutic uses for the polypeptide, and can include enzymes, hormones, and other proteinaceous or non-proteinaceous solutes. In preferred embodiments, the antibody will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated antibody includes antibodies in situ within recombinant cells, because at least one component of the polypeptide natural environment will not be present. Ordinarily, however, isolated polypeptide will be prepared by at least one purification step.

An “ErbB3 cancer detecting agent” refers to an agent that is capable of detecting a mutation associated with an ErbB3 cancer within an ERBB3 nucleic acid sequence or amino acid sequence. Typically, the detecting agent comprises a reagent capable of specifically binding to an ERBB3 sequence. In a preferred embodiment, the reagent is capable of specifically binding to an ErbB3 mutation in an ERBB3 nucleic acid sequence. In one embodiment, the detecting agent comprises a polynucleotide capable of specifically hybridizing to an ERBB3 nucleic acid sequence (e.g., SEQ ID NO:1 or 3). In some embodiments, the polynucleotide is a probe comprising a nucleic acid sequence that specifically hybridizes to an ErbB3 sequence comprising a mutation. In another embodiment, the detecting agent comprises a reagent capable of specifically binding to an ERBB3 amino acid sequence. In another embodiment, the amino acid sequence comprises a mutation as described herein. The detecting agents may further comprise a label. In a preferred embodiment, the ErbB3 cancer detecting agent is an ErbB3 gastro-intestinal cancer detecting agent.

ErbB3 Somatic Mutations

In one aspect, the invention provides methods of detecting the presence or absence of ErbB3 somatic mutations associated with cancer in a sample from a subject, as well as methods of diagnosing and prognosing cancer by detecting the presence or absence of one or more of these somatic mutations in a sample from a subject, wherein the presence of the somatic mutation indicates that the subject has cancer. ErbB3 somatic mutations associated with cancer risk were identified using strategies including genome-wide association studies, modifier screens, and family-based screening.

Somatic mutations or variations for use in the methods of the invention include variations in ErbB3, or the genes encoding this protein. In some embodiments, the somatic mutation is in genomic DNA that encodes a gene (or its regulatory region). In various embodiments, the somatic mutation is a substitution, an insertion, or a deletion in a nucleic acid coding for ErbB3 (SEQ ID NO: 1; Accession No. NM_{—}001982). In an embodiment, the variation is a mutation that results in an amino acid substitution at one or more of M60, G69, M91, V104, Y111, R135, R193, A232, P262, Q281, G284, V295, Q298, G325, T389, R453, M406, V438, D492, K498, V714, Q809, 5846, E928, 51046, R1089, T1164, and D1194 in the amino acid sequence of ErbB3 (SEQ ID NO:2; Accession No. NP_{—}001973). In one embodiment, the substitution is at least one of M60K, G69R, M91I, V104L, V104M, Y111C, R135L, R193*, A232V, P262S, P262H, Q281H, G284R, V295A, Q298*, G325R, T389K, M406K, V438I, R453H, D492H, K498I, V714M, Q809R, S846I, E928G, 51046N, R1089W, T1164A, and D1194E (* indicates a stop codon). In various embodiments, the at least one variation is an amino acid substitution, insertion, truncation, or deletion in ErbB3. In some embodiments, the variation is an amino acid substitution.

Identification of ErbB3 Mutations

In a significant aspect of the present invention, a cluster of ErbB3 amino acid residues has been identified as a mutational hotspot. In particular, it has been found that ErbB3 comprising at least one substitution in the interface between domains I (positions 1 to 213 of SEQ ID NO:2) and II (positions 214 to 284 of SEQ ID NO:2) is indicative of an ErbB3 cancer. In particular, a remarkable extracellular domain (ECD) cluster of somatic mutations has been found at the domain I/II interface determined at least by ErbB3 amino acid residues 104, 232, and 284. In one embodiment, the domain is further determined by amino acid residue 60. In another embodiment, the cluster of somatic mutations includes V104 to L or M; A232 to V; and G284 to R. In one other embodiment, the cluster further includes M60 to K.

In one aspect, the present invention provides methods of determining the presence of gastrointestinal cancer in a subject in need comprising detecting in a biological sample obtained from the subject the presence or absence of an amino acid mutation at the interface, determined by amino acid positions 104, 232 and 284, between domains II and III of human ErbB3. The interface may further be determined by position 60.

Detection of Somatic Mutations

Nucleic acid, as used in any of the detection methods described herein, may be genomic DNA; RNA transcribed from genomic DNA; or cDNA generated from RNA. Nucleic acid may be derived from a vertebrate, e.g., a mammal A nucleic acid is said to be “derived from” a particular source if it is obtained directly from that source or if it is a copy of a nucleic acid found in that source.

Nucleic acid includes copies of the nucleic acid, e.g., copies that result from amplification. Amplification may be desirable in certain instances, e.g., in order to obtain a desired amount of material for detecting variations. The amplicons may then be subjected to a variation detection method, such as those described below, to determine whether a variation is present in the amplicon.

Somatic mutations or variations may be detected by certain methods known to those skilled in the art. Such methods include, but are not limited to, DNA sequencing; primer extension assays, including somatic mutation-specific nucleotide incorporation assays and somatic mutation-specific primer extension assays (e.g., somatic mutation-specific PCR, somatic mutation-specific ligation chain reaction (LCR), and gap-LCR); mutation-specific oligonucleotide hybridization assays (e.g., oligonucleotide ligation assays); cleavage protection assays in which protection from cleavage agents is used to detect mismatched bases in nucleic acid duplexes; analysis of MutS protein binding; electrophoretic analysis comparing the mobility of variant and wild type nucleic acid molecules; denaturing-gradient gel electrophoresis (DGGE, as in, e.g., Myers et al. (1985) Nature 313:495); analysis of RNase cleavage at mismatched base pairs; analysis of chemical or enzymatic cleavage of heteroduplex DNA; mass spectrometry (e.g., MALDI-TOF); genetic bit analysis (GBA); 5′ nuclease assays (e.g., TaqMan™); and assays employing molecular beacons. Certain of these methods are discussed in further detail below.

Detection of variations in target nucleic acids may be accomplished by molecular cloning and sequencing of the target nucleic acids using techniques well known in the art. Alternatively, amplification techniques such as the polymerase chain reaction (PCR) can be used to amplify target nucleic acid sequences directly from a genomic DNA preparation from tumor tissue. The nucleic acid sequence of the amplified sequences can then be determined and variations identified therefrom. Amplification techniques are well known in the art, e.g., the polymerase chain reaction is described in Saiki et al., Science 239:487, 1988; U.S. Pat. Nos. 4,683,203 and 4,683,195.

The ligase chain reaction, which is known in the art, can also be used to amplify target nucleic acid sequences. See, e.g., Wu et al., Genomics 4:560-569 (1989). In addition, a technique known as allele-specific PCR can also modified and used to detect somatic mutations (e.g., substitutions). See, e.g., Ruano and Kidd (1989) Nucleic Acids Research 17:8392; McClay et al. (2002) Analytical Biochem. 301:200-206. In certain embodiments of this technique, a mutation-specific primer is used wherein the 3′ terminal nucleotide of the primer is complementary to (i.e., capable of specifically base-pairing with) a particular variation in the target nucleic acid. If the particular variation is not present, an amplification product is not observed. Amplification Refractory Mutation System (ARMS) can also be used to detect variations (e.g., substitutions). ARMS is described, e.g., in European Patent Application Publication No. 0332435, and in Newton et al., Nucleic Acids Research, 17:7, 1989.

Other methods useful for detecting variations (e.g., substitutions) include, but are not limited to, (1) mutation-specific nucleotide incorporation assays, such as single base extension assays (see, e.g., Chen et al. (2000) Genome Res. 10:549-557; Fan et al. (2000) Genome Res. 10:853-860; Pastinen et al. (1997) Genome Res. 7:606-614; and Ye et al. (2001) Hum. Mut. 17:305-316); (2) mutation-specific primer extension assays (see, e.g., Ye et al. (2001) Hum. Mut. 17:305-316; and Shen et al. Genetic Engineering News, vol. 23, Mar. 15, 2003), including allele-specific PCR; (3) 5′ nuclease assays (see, e.g., De La Vega et al. (2002) BioTechniques 32:S48-S54 (describing the TaqMan® assay); Ranade et al. (2001) Genome Res. 11:1262-1268; and Shi (2001) Clin. Chem. 47:164-172); (4) assays employing molecular beacons (see, e.g., Tyagi et al. (1998) Nature Biotech. 16:49-53; and Mhlanga et al. (2001) Methods 25:463-71); and (5) oligonucleotide ligation assays (see, e.g., Grossman et al. (1994) Nuc. Acids Res. 22:4527-4534; patent application Publication No. US 2003/0119004 A1; PCT International Publication No. WO 01/92579 A2; and U.S. Pat. No. 6,027,889).

Variations may also be detected by mismatch detection methods. Mismatches are hybridized nucleic acid duplexes which are not 100% complementary. The lack of total complementarity may be due to deletions, insertions, inversions, or substitutions. One example of a mismatch detection method is the Mismatch Repair Detection (MRD) assay described, e.g., in Faham et al., Proc. Natl. Acad. Sci. USA 102:14717-14722 (2005) and Faham et al., Hum. Mol. Genet. 10:1657-1664 (2001). Another example of a mismatch cleavage technique is the RNase protection method, which is described in detail in Winter et al., Proc. Natl. Acad. Sci. USA, 82:7575, 1985, and Myers et al., Science 230:1242, 1985. For example, a method of the invention may involve the use of a labeled riboprobe which is complementary to the human wild-type target nucleic acid. The riboprobe and target nucleic acid derived from the tissue sample are annealed (hybridized) together and subsequently digested with the enzyme RNase A which is able to detect some mismatches in a duplex RNA structure. If a mismatch is detected by RNase A, it cleaves at the site of the mismatch. Thus, when the annealed RNA preparation is separated on an electrophoretic gel matrix, if a mismatch has been detected and cleaved by RNase A, an RNA product will be seen which is smaller than the full-length duplex RNA for the riboprobe and the mRNA or DNA. The riboprobe need not be the full length of the target nucleic acid, but can a portion of the target nucleic acid, provided it encompasses the position suspected of having a variation.

In a similar manner, DNA probes can be used to detect mismatches, for example through enzymatic or chemical cleavage. See, e.g., Cotton et al., Proc. Natl. Acad. Sci. USA, 85:4397, 1988; and Shenk et al., Proc. Natl. Acad. Sci. USA, 72:989, 1975. Alternatively, mismatches can be detected by shifts in the electrophoretic mobility of mismatched duplexes relative to matched duplexes. See, e.g., Cariello, Human Genetics, 42:726, 1988. With either riboprobes or DNA probes, the target nucleic acid suspected of comprising a variation may be amplified before hybridization. Changes in target nucleic acid can also be detected using Southern hybridization, especially if the changes are gross rearrangements, such as deletions and insertions.

Restriction fragment length polymorphism (RFLP) probes for the target nucleic acid or surrounding marker genes can be used to detect variations, e.g., insertions or deletions. Insertions and deletions can also be detected by cloning, sequencing and amplification of a target nucleic acid. Single stranded conformation polymorphism (SSCP) analysis can also be used to detect base change variants of an allele. See, e.g. Orita et al., Proc. Natl. Acad. Sci. USA 86:2766-2770, 1989, and Genomics, 5:874-879, 1989. SSCP can be modified for the detection of ErbB3 somatic mutations. SSCP identifies base differences by alteration in electrophoretic migration of single stranded PCR products. Single-stranded PCR products can be generated by heating or otherwise denaturing double stranded PCR products. Single-stranded nucleic acids may refold or form secondary structures that are partially dependent on the base sequence. The different electrophoretic mobilities of single-stranded amplification products are related to base-sequence differences at SNP positions. Denaturing gradient gel electrophoresis (DGGE) differentiates SNP alleles based on the different sequence-dependent stabilities and melting properties inherent in polymorphic DNA and the corresponding differences in electrophoretic migration patterns in a denaturing gradient gel.

Somatic mutations or variations may also be detected with the use of microarrays. A microarray is a multiplex technology that typically uses an arrayed series of thousands of nucleic acid probes to hybridize with, e.g, a cDNA or cRNA sample under high-stringency conditions. Probe-target hybridization is typically detected and quantified by detection of fluorophore-, silver-, or chemiluminescence-labeled targets to determine relative abundance of nucleic acid sequences in the target. In typical microarrays, the probes are attached to a solid surface by a covalent bond to a chemical matrix (via epoxy-silane, amino-silane, lysine, polyacrylamide or others). The solid surface is for example, glass, a silicon chip, or microscopic beads. Various microarrays are commercially available, including those manufactured, for example, by Affymetrix, Inc. and Illumina, Inc.

Another method for the detection of somatic mutations is based on mass spectrometry. Mass spectrometry takes advantage of the unique mass of each of the four nucleotides of DNA. The potential mutation-containing ErbB3 nucleic acids can be unambiguously analyzed by mass spectrometry by measuring the differences in the mass of nucleic acids having a somatic mutation. MALDI-TOF (Matrix Assisted Laser Desorption Ionization-Time of Flight) mass spectrometry technology is useful for extremely precise determinations of molecular mass, such the nucleic acids containing a somatic mutation. Numerous approaches to nucleic acid analysis have been developed based on mass spectrometry. Exemplary mass spectrometry-based methods include primer extension assays, which can also be utilized in combination with other approaches, such as traditional gel-based formats and microarrays.

Sequence-specific ribozymes (U.S. Pat. No. 5,498,531) can also be used to detect somatic mutations based on the development or loss of a ribozyme cleavage site. Perfectly matched sequences can be distinguished from mismatched sequences by nuclease cleavage digestion assays or by differences in melting temperature. If the mutation affects a restriction enzyme cleavage site, the mutation can be identified by alterations in restriction enzyme digestion patterns, and the corresponding changes in nucleic acid fragment lengths determined by gel electrophoresis.

In other embodiments of the invention, protein-based detection techniques are used to detect variant proteins encoded by the genes having genetic variations as disclosed herein. Determination of the presence of the variant form of the protein can be carried out using any suitable technique known in the art, for example, electrophoresis (e.g, denaturing or non-denaturing polyacrylamide gel electrophoresis, 2-dimensional gel electrophoresis, capillary electrophoresis, and isoelectrofocusing), chromatrography (e.g., sizing chromatography, high performance liquid chromatography (HPLC), and cation-exchange HPLC), and mass spectroscopy (e.g., MALDI-TOF mass spectroscopy, electrospray ionization (ESI) mass spectroscopy, and tandem mass spectroscopy). See, e.g., Ahrer and Jungabauer (2006) J. Chromatog. B. Analyt. Technol. Biomed. Life Sci. 841: 110-122; and Wada (2002) J. Chromatog. B. 781: 291-301). Suitable techniques may be chosen based in part upon the nature of the variation to be detected. For example, variations resulting in amino acid substitutions where the substituted amino acid has a different charge than the original amino acid, can be detected by isoelectric focusing. Isoelectric focusing of the polypeptide through a gel having a pH gradient at high voltages separates proteins by their pH. The pH gradient gel can be compared to a simultaneously run gel containing the wild-type protein. In cases where the variation results in the generation of a new proteolytic cleavage site, or the abolition of an existing one, the sample may be subjected to proteolytic digestion followed by peptide mapping using an appropriate electrophoretic, chromatographic or, or mass spectroscopy technique. The presence of a variation may also be detected using protein sequencing techniques such as Edman degradation or certain forms of mass spectroscopy.

Methods known in the art using combinations of these techniques may also be used. For example, in the HPLC-microscopy tandem mass spectrometry technique, proteolytic digestion is performed on a protein, and the resulting peptide mixture is separated by reversed-phase chromatographic separation. Tandem mass spectrometry is then performed and the data collected therefrom is analyzed. (Gatlin et al. (2000) Anal. Chem., 72:757-763). In another example, nondenaturing gel electrophoresis is combined with MALDI mass spectroscopy (Mathew et al. (2011) Anal. Biochem. 416: 135-137).

In some embodiments, the protein may be isolated from the sample using a reagent, such as antibody or peptide that specifically binds the protein, and then further analyzed to determine the presence or absence of the genetic variation using any of the techniques disclosed above.

Alternatively, the presence of the variant protein in a sample may be detected by immunoaffinity assays based on antibodies specific to proteins having genetic variations according to the present invention, that is, antibodies which specifically bind to the protein having the variation, but not to a form of the protein which lacks the variation. Such antibodies can be produced by any suitable technique known in the art. Antibodies can be used to immunoprecipitate specific proteins from solution samples or to immunoblot proteins separated by, e.g., polyacrylamide gels Immunocytochemical methods can also be used in detecting specific protein variants in tissues or cells. Other well known antibody-based techniques can also be used including, e.g., enzyme-linked immunosorbent assay (ELISA), radioimmuno-assay (RIA), immunoradiometric assays (IRMA) and immunoenzymatic assays (IEMA), including sandwich assays using monoclonal or polyclonal antibodies. See e.g., U.S. Pat. Nos. 4,376,110 and 4,486,530.

Identification of Genetic Markers

The relationship between somatic mutations and germline mutations has investigated in cancer (see e.g. Zauber et al. J. Pathol. 2003 February; 199(2):146-51). The ErbB3 somatic mutations disclosed herein are useful for identifying genetic markers associated with the development of cancer. For example, the somatic mutations disclosed herein can be used to identify single nucleotide polymorphisms (SNPs) in the germline and any additional SNPs that are in linkage disequilibrium. Indeed, any additional SNP in linkage disequilibrium with a first SNP associated with cancer will be associated with cancer. Once the association has been demonstrated between a given SNP and cancer, the discovery of additional SNPs associated with cancer can be of great interest in order to increase the density of SNPs in this particular region.

Methods for identifying additional SNPs and conducting linkage disequilibrium analysis are well known in the art. For example, identification of additional SNPs in linkage disequilibrium with the SNPs disclosed herein can involve the steps of: (a) amplifying a fragment from the genomic region comprising or surrounding a first SNP from a plurality of individuals; (b) identifying of second SNPs in the genomic region harboring or surrounding said first SNP; (c) conducting a linkage disequilibrium analysis between said first SNP and second SNPs; and (d) selecting said second SNPs as being in linkage disequilibrium with said first marker. This method may be modified to include certain steps preceding step (a), such as amplifying a fragment from the genomic region comprising or surrounding a somatic mutation from a plurality of individuals, and identifying SNPs in the genomic region harboring or surrounding said somatic mutation.

ErbB3 Cancer Detecting Agents

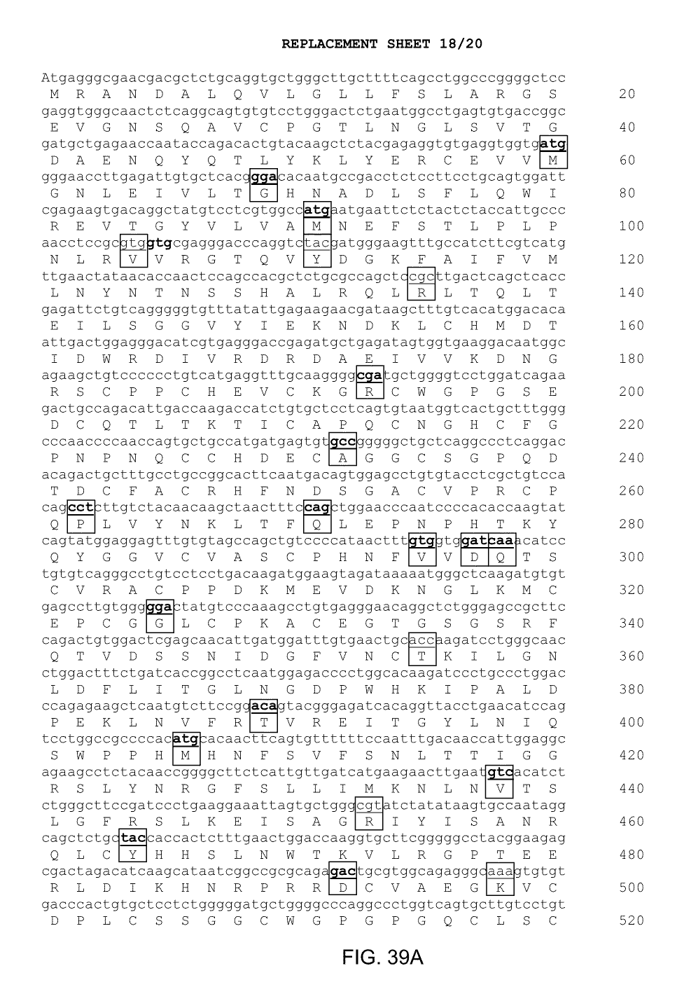

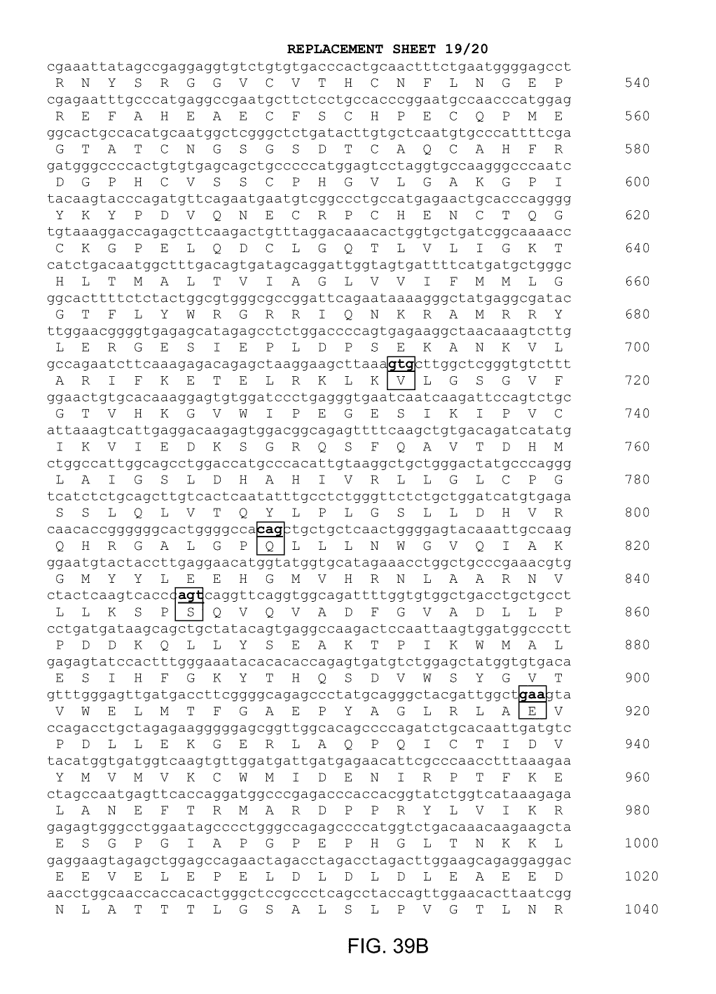

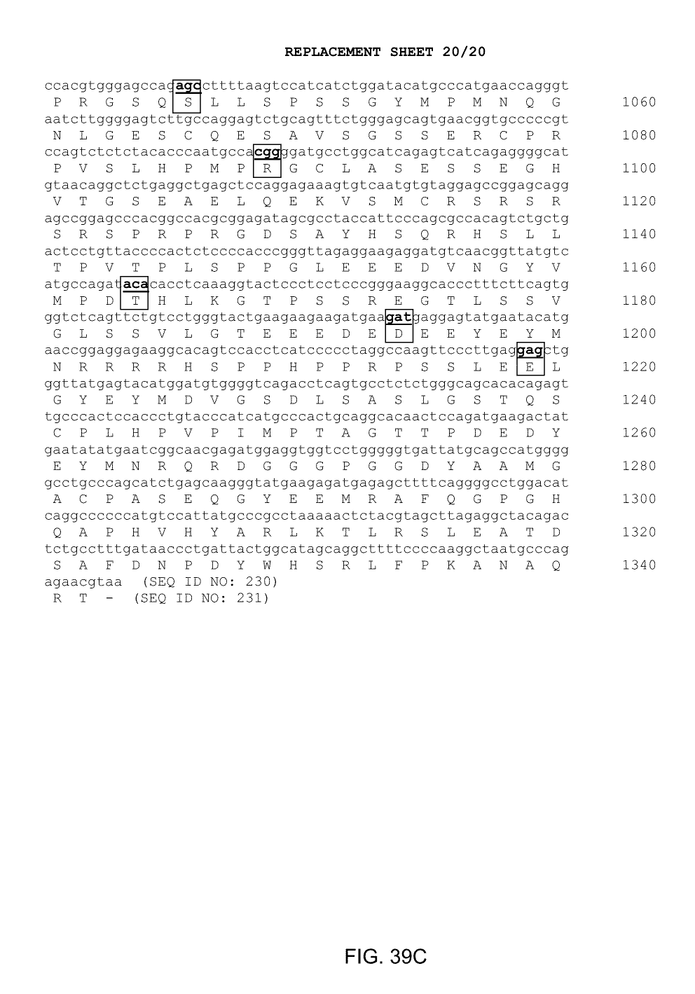

In one aspect, the present invention provides ErbB3 cancer detecting agents. In one embodiment, the detecting agent comprises a reagent capable of specifically binding to an ErbB3 sequence shown in FIG. 39A-C (amino acid sequence of SEQ ID NO: 2 or nucleic acid sequence of SEQ ID NO:3). In another embodiment, the detecting agent comprises a polynucleotide capable of specifically hybridizing to an ERBB3 nucleic acid sequence shown in FIG. 2 (SEQ ID NO: 1) or FIG. 39A-C (SEQ ID NO:3). In a preferred embodiment, the polynucleotide comprises a nucleic acid sequence that specifically hybridizes to an ErbB3 nucleic acid sequence comprising a mutation shown in FIG. 39A-C (SEQ ID NO:3).

In another aspect, the ErbB3 cancer detecting agents comprise a polynucleotide having a particular formula. In one embodiment, the polynucleotide formula is

5′Xₐ—Y—Z_{b}3′ Formula I

, wherein

X is any nucleic acid and a is between about 0 and about 250 (i.e., in the 5′ direction);

Y represents an ErbB3 mutation codon; and

Z is any nucleic acid and b is between about 0 and about 250 (i.e., in the 3′ direction).

In another embodiment, a or b is about 250 or less in the 5′ (if a) or 3′ (if b) direction. In some embodiments, a or b is between about 0 and about 250, a or b is between about 0 and about 245, about 0 and about 240, between about 0 and about 230, between about 0 and about 220, between about 0 and about 210, between about 0 and about 200, between about 0 and about 190, between about 0 and about 180, between about 0 and about 170, between about 0 and about 160, between about 0 and about 150, between about 0 and about 140, between about 0 and about 130, between about 0 and about 120, between about 0 and about 110, between about 0 and about 100, between about 0 and about 90, between about 0 and about 80, between about 0 and about 70, between about 0 and about 60, between about 0 and about 50, between about 0 and about 45, between about 0 and about 40, between about 0 and about 35, between about 0 and about 30, between about 0 and about 25, between about 0 and about 20, between about 0 and about 15, between about 0 and about 10, or between about 0 and about 5.

In one other embodiment, a or b is about 35 or less. In some embodiments, a or b is between about 0 and about 35, between about 0 and about 34, between about 0 and about 33, between about 0 and about 32, between about 0 and about 31, between about 0 and about 30, between about 0 and about 29, between about 0 and about 28, between about 0 and about 27,

between about 0 and about 26, between about 0 and about 25, between about 0 and about 24, between about 0 and about 23, between about 0 and about 22, between about 0 and about 21, between about 0 and about 20, between about 0 and about 19, between about 0 and about 18, between about 0 and about 17, between about 0 and about 16, between about 0 and about 15, between about 0 and about 14, between about 0 and about 13, between about 0 and about 12, between about 0 and about 11, between about 0 and about 10, between about 0 and about 9, between about 0 and about 8, between about 0 and about 7, between about 0 and about 6, between about 0 and about 5, between about 0 and about 4, between about 0 and about 3, or between about 0 and about 2.

In one other embodiment, the polynucleotide hybridizes to an ErbB3 nucleic acid sequence encoding an amino acid at position 60 of SEQ ID NO:2, wherein Y is selected from the group consisting of AAA and AAG. This corresponds to the M60K mutation associated with colon cancer.

In one other embodiment, the polynucleotide hybridizes to an ErbB3 nucleic acid sequence encoding an amino acid at position 104 of SEQ ID NO:2, wherein Y is selected from the group consisting of ATG, CTT, CTC, CTA, CTG, TTA, and TTG. This corresponds to the V104M or V104L mutation associated with colon, gastric, ovarian, and breast cancer.

In one other embodiment, the polynucleotide hybridizes to an ErbB3 nucleic acid sequence encoding an amino acid at position 111 of SEQ ID NO:2, wherein Y is selected from the group consisting of TGT and TGC. This corresponds to the Y111C mutation associated with gastric cancer.

In one other embodiment, the polynucleotide hybridizes to an ErbB3 nucleic acid sequence encoding an amino acid at position 135 of SEQ ID NO:2, wherein Y is selected from the group consisting of CTT, CTC, CTA, CTG, TTA, and TTG. This corresponds to the R135L mutation associated with gastric cancer.

In one other embodiment, the polynucleotide hybridizes to an ErbB3 nucleic acid sequence encoding an amino acid at position 193 of SEQ ID NO:2, wherein Y is selected from the group consisting of TAA, TAG, and TGA. This corresponds to the R193* (where * is a stop codon) mutation associated with colon cancer.

In one other embodiment, the polynucleotide hybridizes to an ErbB3 nucleic acid sequence encoding an amino acid at position 232 of SEQ ID NO:2, wherein Y is selected from the group consisting of GTT, GTC, GTA, and GTG. This corresponds to the A232V mutation associated with gastric cancer.

In one other embodiment, the polynucleotide hybridizes to an ErbB3 nucleic acid sequence encoding an amino acid at position 262 of SEQ ID NO:2, wherein Y is selected from the group consisting of CAT, CAC, TCT, TCC, TCA, TCG, AGT, and AGC. This corresponds to the P262H or P262S mutation associated with colon and/or gastric cancer.

In one other embodiment, the polynucleotide hybridizes to an ErbB3 nucleic acid sequence encoding an amino acid at position 284 of SEQ ID NO:2, wherein Y is selected from the group consisting of CGT, CGC, CGA, CGG, AGA, and AGG. This corresponds to the G284R mutation associated with colon or lung (NSCLC adenocarcinoma.) cancer.

In one other embodiment, the polynucleotide hybridizes to an ErbB3 nucleic acid sequence encoding an amino acid at position 295 of SEQ ID NO:2, wherein Y is selected from the group consisting of GCT, GCC, GCA, and GCG. This corresponds to the V295A mutation associated with colon cancer.

In one other embodiment, the polynucleotide hybridizes to an ErbB3 nucleic acid sequence encoding an amino acid at position 325 of SEQ ID NO:2, wherein Y is selected from the group consisting of CGT, CGC, CGA, CGG, AGA, and AGG. This corresponds to the G325R mutation associated with colon cancer.

In one other embodiment, the polynucleotide hybridizes to an ErbB3 nucleic acid sequence encoding an amino acid at position 406 of SEQ ID NO:2, wherein Y is selected from the group consisting of ACT, ACC, ACA, ACG, AAA and AAG. This corresponds to the M406K or M406T mutation associated with gastric cancer.

In one other embodiment, the polynucleotide hybridizes to an ErbB3 nucleic acid sequence encoding an amino acid at position 453 of SEQ ID NO:2, wherein Y is selected from the group consisting of CAT and CAC. This corresponds to the R453H mutation associated with gastric cancer.

In one other embodiment, the polynucleotide hybridizes to an ErbB3 nucleic acid sequence encoding an amino acid at position 498 of SEQ ID NO:2, wherein Y is selected from the group consisting of ATT, ATC, and ATA. This corresponds to the K₄₉₈I mutation associated with gastric cancer.

In one other embodiment, the polynucleotide hybridizes to an ErbB3 nucleic acid sequence encoding an amino acid at position 809 of SEQ ID NO:2, wherein Y is selected from the group consisting of CGT, CGC, CGA, CGG, AGA, and AGG. This corresponds to the Q809R mutation associated with gastric cancer.

In one other embodiment, the polynucleotide hybridizes to an ErbB3 nucleic acid sequence encoding an amino acid at position 846 of SEQ ID NO:2, wherein Y is selected from the group consisting of ATT, ATC, and ATA. This corresponds to the S846I mutation associated with colon cancer.

In one other embodiment, the polynucleotide hybridizes to an ErbB3 nucleic acid sequence encoding an amino acid at position 928 of SEQ ID NO:2, wherein Y is selected from the group consisting of GGT, GGC, GGA, and GGG. This corresponds to the E928G mutation associated with gastric cancer and breast cancer.

In one other embodiment, the polynucleotide hybridizes to an ErbB3 nucleic acid sequence encoding an amino acid at position 1089 of SEQ ID NO:2, wherein Y is TGG. This corresponds to the R1089W mutation associate with gastric cancer.

In one other embodiment, the polynucleotide hybridizes to an ErbB3 nucleic acid sequence encoding an amino acid at position 1164 of SEQ ID NO:2, wherein Y is selected from the group consisting of GCT, GCC, GCA, and GCG. This corresponds to the T1164A mutation associated with colon cancer.

In one other embodiment, the polynucleotide hybridizes to an ErbB3 nucleic acid sequence encoding an amino acid at position 492 of SEQ ID NO:2, wherein Y is selected from the group consisting of CAT and CAC. This corresponds to the D492H mutation associated with lung (NSCLC adenocarcinoma) cancer.

In one other embodiment, the polynucleotide hybridizes to an ErbB3 nucleic acid sequence encoding an amino acid at position 714 of SEQ ID NO:2, wherein Y is ATG. This corresponds to the V714M mutation associated with lung (NSCLC squamous carcinoma) cancer.

Diagnosis, Prognosis and Treatment of Cancer

The invention provides methods for the diagnosis or prognosis of cancer in a subject by detecting the presence in a sample from the subject of one or more somatic mutations or variations associated with cancer as disclosed herein. Somatic mutations or variations for use in the methods of the invention include variations in ErbB3, or the genes encoding this protein. In some embodiments, the somatic mutation is in genomic DNA that encodes a gene (or its regulatory region). In various embodiments, the somatic mutation is a substitution, an insertion, or a deletion in the gene coding for ErbB3. In an embodiment, the variation is a mutation that results in an amino acid substitution at one or more of M60, G69, M91, V104, Y111, R135, R193, A232, P262, Q281, G284, V295, Q298, G325, T389, M406, V438, R453, D492, K498, V714, Q809, 5846, E928, 51046, R1089, T1164, and D1194 in the amino acid sequence of ErbB3 (SEQ ID NO:2). In one embodiment, the substitution is at least one of M60K, G69R, M91I, V104L, V104M, Y111C, R135L, R193*, A232V, P262S, P262H, Q281H, G284R, V295A, Q298*, G325R, T389K, M406K, V438I, R453H, D492H, K498I, V714M, Q809R, S846I, E928G, S1046N, R1089W, T1164A, and D1194E (* indicates a stop codon) in the amino acid sequence of ErbB3 (SEQ ID NO:2). In one embodiment, the mutation indicates the presence of an ErbB3 cancer selected from the group consisting of gastric, colon, esophageal, rectal, cecum, colorectal, non-small-cell lung (NSCLC) adenocarinoma, NSCLC (Squamous carcinoma), renal carcinoma, melanoma, ovarian, lung large cell, small-cell lung cancer (SCLC), hepatocellular (HCC), lung cancer, and pancreatic cancer.

In one other embodiment, the variation is a mutation that results in an amino acid substitution at one or more of M60, V104, Y111, R153, R193, A232, P262, V295, G325, M406, R453, D492, K498, V714, Q809, R1089, and T1164 in the amino acid sequence of ErbB3 (SEQ ID NO:2). In another embodiment, the substitution is at least one of M60K, V104M, V104L, Y111C, R153L, R193*, A232V, P262S, P262H, V295A, G325R, M406K, R453H, D492H, K₄₉₈I, V714M, Q809R, R1089W, and D1194E (* indicates a stop codon) in the amino acid sequence of ErbB3 (SEQ ID NO:2). In one embodiment, the mutation indicates the presence of an ErbB3 cancer selected from the group consisting of gastric, colon, esophageal, rectal, cecum, colorectal, non-small-cell lung (NSCLC) adenocarinoma, NSCLC (Squamous carcinoma), renal carcinoma, melanoma, ovarian, lung large cell, small-cell lung cancer (SCLC), hepatocellular (HCC), lung cancer, and pancreatic cancer.

In one other embodiment, the variation is a mutation that results in an amino acid substitution at one or more of V104, Y111, R153, A232, P262, G284, T389, R453, K498, and Q809 in the amino acid sequence of ErbB3 (SEQ ID NO:2). In another embodiment, the substitution is at least one of V104L, V104M, Y111C, R153L, A232V, P262S, P262H, G284R, T389K, R453H, K498I, and Q809R in the amino acid sequence of ErbB3 (SEQ ID NO:2). In one embodiment, the ErbB3 mutation indicates the presence of gastrointestinal cancer. In another embodiment, a gastrointestinal cancer is one or more of gastric, colon, esophageal, rectal, cecum, and colorectal cancer.

In one embodiment, the ErbB3 substitution is at M60. In another embodiment, the substitution is M60K. In one other embodiment, the mutation indicates the presence of colon cancer.

In one embodiment, the ErbB3 substitution is at V104. In another embodiment, the substitution is V104L or V104M. In one other embodiment, the mutation indicates the presence of gastric cancer or colon cancer.

In one embodiment, the ErbB3 substitution is at V111. In another embodiment, the substitution is V111C. In one other embodiment, the mutation indicates the presence of gastric cancer.

In one embodiment, the ErbB3 substitution is at R135. In another embodiment, the substitution is R135L. In one other embodiment, the mutation indicates the presence of gastric cancer.

In one embodiment, the ErbB3 substitution is at R193. In another embodiment, the substitution is R193*. In one other embodiment, the mutation indicates the presence of colon cancer.

In one embodiment, the ErbB3 substitution is at A232. In another embodiment, the substitution is A232V. In one other embodiment, the mutation indicates the presence of gastric cancer.

In one embodiment, the ErbB3 substitution is at P262. In another embodiment, the substitution is P262S or P262H. In one other embodiment, the mutation indicates the presence of colon cancer or gastric cancer.

In one embodiment, the ErbB3 substitution is at G284. In another embodiment, the substitution is G284R. In one other embodiment, the mutation indicates the presence of lung cancer (non-small-cell lung (NSCLC) adenocarinoma) or colon cancer.

In one embodiment, the ErbB3 substitution is at V295. In another embodiment, the substitution is V295A. In one other embodiment, the mutation indicates the presence of colon cancer.

In one embodiment, the ErbB3 substitution is at G325. In another embodiment, the substitution is G325R. In one other embodiment, the mutation indicates the presence of colon cancer.

In one embodiment, the ErbB3 substitution is at M406. In another embodiment, the substitution is M406K. In one other embodiment, the mutation indicates the presence of gastric cancer.

In one embodiment, the ErbB3 substitution is at R453. In another embodiment, the substitution is R453H. In one other embodiment, the mutation indicates the presence of gastric cancer or colon cancer.

In one embodiment, the ErbB3 substitution is at K498. In another embodiment, the substitution is K498I. In one other embodiment, the mutation indicates the presence of gastric cancer.

In one embodiment, the ErbB3 substitution is at D492. In another embodiment, the substitution is D492H. In one other embodiment, the mutation indicates the presence of lung cancer (non-small-cell lung (NSCLC) adenocarinoma).

In one embodiment, the ErbB3 substitution is at V714. In another embodiment, the substitution is V714M. In one other embodiment, the mutation indicates the presence of lung cancer (non-small-cell lung (NSCLC) squamous carcinoma).

In one embodiment, the ErbB3 substitution is at Q809. In another embodiment, the substitution is Q809R. In one other embodiment, the mutation indicates the presence of gastric cancer.

In one embodiment, the ErbB3 substitution is at S846. In another embodiment, the substitution is S846I. In one other embodiment, the mutation indicates the presence of colon cancer.

In one embodiment, the ErbB3 substitution is at R1089. In another embodiment, the substitution is R1089W. In one other embodiment, the mutation indicates the presence of gastric cancer.

In one embodiment, the ErbB3 substitution is at T1164. In another embodiment, the substitution is T1164A. In one other embodiment, the mutation indicates the presence of colon cancer.

In various embodiments, the at least one variation is an amino acid substitution, insertion, truncation, or deletion in ErbB3. In some embodiments, the variation is an amino acid substitution. Any one or more of these variations may be used in any of the methods of detection, diagnosis and prognosis described below.

In an embodiment, the invention provides a method for detecting the presence or absence of a somatic mutation indicative of cancer in a subject, comprising: (a) contacting a sample from the subject with a reagent capable of detecting the presence or absence of a somatic mutation in an ErbB3 gene; and (b) determining the presence or absence of the mutation, wherein the presence of the mutation indicates that the subject is afflicted with, or at risk of developing, cancer.

The reagent for use in the method may be an oligonucleotide, a DNA probe, an RNA probe, and a ribozyme. In some embodiments, the reagent is labeled. Labels may include, for example, radioisotope labels, fluorescent labels, bioluminescent labels or enzymatic labels. Radionuclides that can serve as detectable labels include, for example, I-131, I-123, I-125, Y-90, Re-188, Re-186, At-211, Cu-67, Bi-212, and Pd-109.

Also provided is a method for detecting a somatic mutation indicative of cancer in a subject, comprising: determining the presence or absence of a somatic mutation in an ErbB3 gene in a biological sample from a subject, wherein the presence of the mutation indicates that the subject is afflicted with, or at risk of developing, cancer. In various embodiments of the method, detection of the presence of the one or more somatic mutations is carried out by a process selected from the group consisting of direct sequencing, mutation-specific probe hybridization, mutation-specific primer extension, mutation-specific amplification, mutation-specific nucleotide incorporation, 5′ nuclease digestion, molecular beacon assay, oligonucleotide ligation assay, size analysis, and single-stranded conformation polymorphism. In some embodiments, nucleic acids from the sample are amplified prior to determining the presence of the one or more mutations.

The invention further provides a method for diagnosing or prognosing cancer in a subject, comprising: (a) contacting a sample from the subject with a reagent capable of detecting the presence or absence of a somatic mutation in an ErbB3 gene; and (b) determining the presence or absence of the mutation, wherein the presence of the mutation indicates that the subject is afflicted with, or at risk of developing, cancer.

The invention further provides a method of diagnosing or prognosing cancer in a subject, comprising: determining the presence or absence of a somatic mutation in an ErbB3 gene in a biological sample from a subject, wherein the presence of the genetic variation indicates that the subject is afflicted with, or at risk of developing, cancer.

The invention also provides a method of diagnosing or prognosing cancer in a subject, comprising: (a) obtaining a nucleic-acid containing sample from the subject, and (b) analyzing the sample to detect the presence of at least one somatic mutation in an ErbB3 gene, wherein the presence of the genetic variation indicates that the subject is afflicted with, or at risk of developing, cancer.

In some embodiments, the method of diagnosis or prognosis further comprises subjecting the subject to one or more additional diagnostic tests for cancer, for example, screening for one or more additional markers, or subjecting the subject to imaging procedures.

It is further contemplated that any of the above methods may further comprise treating the subject for cancer based on the results of the method. In some embodiments, the above methods further comprise detecting in the sample the presence of at least one somatic mutation. In an embodiment, the presence of a first somatic mutation together with the presence of at least one additional somatic mutation is indicative of an increased risk of cancer compared to a subject having the first somatic mutation and lacking the presence of the at least one additional somatic mutation.

Also provided is a method of identifying a subject having an increased risk of the diagnosis of cancer, comprising: (a) determining the presence or absence of a first somatic mutation in an ErbB3 gene in a biological sample from a subject; and (b) determining the presence or absence of at least one additional somatic mutation, wherein the presence of the first and at least one additional somatic mutations indicates that the subject has an increased risk of the diagnosis of cancer as compared to a subject lacking the presence of the first and at least one additional somatic mutation.

Also provided is a method of aiding diagnosis and/or prognosis of a sub-phenotype of cancer in a subject, the method comprising detecting in a biological sample derived from the subject the presence of a somatic mutation in a gene encoding ErbB3. In an embodiment, the somatic mutation results in the amino acid substitution G284R in the amino acid sequence of ErbB3 (SEQ ID NO: 2), and the sub-phenotype of cancer is characterized at least in part by HER ligand-independent signaling of a cell expressing the G284R mutant ErbB3. In another embodiment, the somatic mutation results in the amino acid substitution Q809R in the amino acid sequence of ErbB3 (SEQ ID NO: 2), and the sub-phenotype of cancer is characterized at least in part by HER ligand-independent signaling of a cell expressing the Q809R mutant ErbB3.

The invention further provides a method of predicting the response of a subject to a cancer therapeutic agent that targets an ErbB receptor, comprising detecting in a biological sample obtained from the subject a somatic mutation that results in an amino acid variation in the amino acid sequence of ErbB3 (SEQ ID NO: 2), wherein the presence of the somatic mutation is indicative of a response to a therapeutic agent that targets an ErbB receptor. In an embodiment, the therapeutic agent is an ErbB antagonist or binding agent, for example, an anti-ErbB antibody.

A biological sample for use in any of the methods described above may be obtained using certain methods known to those skilled in the art. Biological samples may be obtained from vertebrate animals, and in particular, mammals. In certain embodiments, a biological sample comprises a cell or tissue. Variations in target nucleic acids (or encoded polypeptides) may be detected from a tissue sample or from other body samples such as blood, serum, urine, sputum, saliva, mucosa, and tissue. By screening such body samples, a simple early diagnosis can be achieved for diseases such as cancer. In addition, the progress of therapy can be monitored more easily by testing such body samples for variations in target nucleic acids (or encoded polypeptides). In some embodiments, the biological sample is obtained from an individual suspected of having cancer.

Subsequent to the determination that a subject, or biological sample obtained from the subject, comprises a somatic mutation disclosed herein, it is contemplated that an effective amount of an appropriate cancer therapeutic agent may be administered to the subject to treat cancer in the subject.

Also provided are methods for aiding in the diagnosis of cancer in a mammal by detecting the presence of one or more variations in nucleic acid comprising a somatic mutation in ErbB3, according to the method described above.

In another embodiment, a method is provided for predicting whether a subject with cancer will respond to a therapeutic agent by determining whether the subject comprises a somatic mutation in ErbB3, according to the method described above.

Also provided are methods for assessing predisposition of a subject to develop cancer by detecting presence or absence in the subject of a somatic mutation in ErbB3.

Also provided are methods of sub-classifying cancer in a mammal, the method comprising detecting the presence of a somatic mutation in ErbB3.

Also provided are methods of identifying a therapeutic agent effective to treat cancer in a patient subpopulation, the method comprising correlating efficacy of the agent with the presence of a somatic mutation in ErbB3.

Additional methods provide information useful for determining appropriate clinical intervention steps, if and as appropriate. Therefore, in one embodiment of a method of the invention, the method further comprises a clinical intervention step based on results of the assessment of the presence or absence of an ErbB3 somatic mutation associated with cancer as disclosed herein. For example, appropriate intervention may involve prophylactic and treatment steps, or adjustment(s) of any then-current prophylactic or treatment steps based on genetic information obtained by a method of the invention.

As would be evident to one skilled in the art, in any method described herein, while detection of presence of a somatic mutation would positively indicate a characteristic of a disease (e.g., presence or subtype of a disease), non-detection of a somatic mutation would also be informative by providing the reciprocal characterization of the disease.

Still further methods include methods of treating cancer in a mammal, comprising the steps of obtaining a biological sample from the mammal, examining the biological sample for the presence or absence of an ErbB3 somatic mutation as disclosed herein, and upon determining the presence or absence of the mutation in said tissue or cell sample, administering an effective amount of an appropriate therapeutic agent to said mammal Optionally, the methods comprise administering an effective amount of a targeted cancer therapeutic agent to said mammal.

Also provided are methods of treating cancer in a subject in whom an ErbB3 somatic mutation is known to be present, the method comprising administering to the subject a therapeutic agent effective to treat cancer.

Also provided are methods of treating a subject having cancer, the method comprising administering to the subject a therapeutic agent previously shown to be effective to treat said cancer in at least one clinical study wherein the agent was administered to at least five human subjects who each had an ErbB3 somatic mutation. In one embodiment, the at least five subjects had two or more different somatic mutations in total for the group of at least five subjects. In one embodiment, the at least five subjects had the same somatic mutations for the entire group of at least five subjects.

Also provided are methods of treating a cancer subject who is of a specific cancer patient subpopulation comprising administering to the subject an effective amount of a therapeutic agent that is approved as a therapeutic agent for said subpopulation, wherein the subpopulation is characterized at least in part by association with an ErbB3 somatic mutation.

In one embodiment, the subpopulation is of European ancestry. In one embodiment, the invention provides a method comprising manufacturing a cancer therapeutic agent, and packaging the agent with instruction to administer the agent to a subject who has or is believed to have cancer and who has an ErbB3 somatic mutation.

Also provided are methods for selecting a patient suffering from cancer for treatment with a cancer therapeutic agent comprising detecting the presence of an ErbB3 somatic mutation.

A therapeutic agent for the treatment of cancer may be incorporated into compositions, which in some embodiments are suitable for pharmaceutical use. Such compositions typically comprise the peptide or polypeptide, and an acceptable carrier, for example one that is pharmaceutically acceptable. A “pharmaceutically acceptable carrier” includes any and all solvents, dispersion media, coatings, antibacterial and antifungal agents, isotonic and absorption delaying agents, and the like, compatible with pharmaceutical administration (Gennaro, Remington: The science and practice of pharmacy. Lippincott, Williams & Wilkins, Philadelphia, Pa. (2000)). Examples of such carriers or diluents include, but are not limited to, water, saline, Finger's solutions, dextrose solution, and 5% human serum albumin. Liposomes and non-aqueous vehicles such as fixed oils may also be used. Except when a conventional media or agent is incompatible with an active compound, use of these compositions is contemplated. Supplementary active compounds can also be incorporated into the compositions.

A therapeutic agent of the invention (and any additional therapeutic agent for the treatment of cancer) can be administered by any suitable means, including parenteral, intrapulmonary, intrathecal and intranasal, and, if desired for local treatment, intralesional administration. Parenteral infusions include, e.g., intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Effective dosages and schedules for administering cancer therapeutic agents may be determined empirically, and making such determinations is within the skill in the art. Single or multiple dosages may be employed. When in vivo administration of a cancer therapeutic agent is employed, normal dosage amounts may vary from about 10 ng/kg to up to 100 mg/kg of mammal body weight or more per day, preferably about 1 μg/kg/day to 10 mg/kg/day, depending upon the route of administration. Guidance as to particular dosages and methods of delivery is provided in the literature; see, for example, U.S. Pat. Nos. 4,657,760; 5,206,344; or 5,225,212.

One aspect of the invention provides a method of treating an individual having an HER3/ErbB3 cancer identified by one or more of the somatic mutations described herein. In one embodiment, the method comprises the step of administering to the individual an effective amount of a HER inhibitor. In another embodiment, the HER inhibitor is an antibody which binds to a HER receptor. In a preferred embodiment, the antibody binds to an ErbB3 receptor. In one embodiment, the HER antibody is a multispecific antibody comprising an antigen-binding domain that specifically binds to HER3 and at least one additional HER receptor, such as those described in Fuh et al. WO10/108,127 incorporated herein by reference in its entirety. In one embodiment, the ErbB3 cancer treated by the HER inhibitor comprises cells that express HER3. In one embodiment, the cancer treated by the HER inhibitor is gastric, colon, esophageal, rectal, cecum, colorectal, non-small-cell lung (NSCLC) adenocarinoma, NSCLC (Squamous carcinoma), renal carcinoma, melanoma, ovarian, lung large cell, small-cell lung cancer (SCLC), hepatocellular (HCC), lung cancer, and pancreatic cancer.

Another aspect of the invention provides for a method of inhibiting a biological activity of a HER receptor in an individual comprising administering to the individual an effective amount of a HER inhibitor. In one embodiment, the HER receptor is a HER3 receptor expressed by cancer cells in the individual. In another embodiment, the HER inhibitor is a HER antibody comprising an antigen-binding domain that specifically binds to at least HER3.

One aspect of the invention provides for a HER antibody for use as a medicament. Another aspect of the invention provides for a HER antibody for use in the manufacture of a medicament. The medicament can be used, in one embodiment, to treat an ErbB3/HER3 cancer identified by one or more of the somatic mutations described herein. In one embodiment, the medicament is for inhibiting a biological activity of the HER3 receptor. In one embodiment, the HER antibody comprises an antigen-binding domain that specifically binds to HER3, or to HER3 and at least one additional HER receptor.

In another aspect, the present invention provides several different types of suitable HER inhibitor for the methods of treatment. In one embodiment, the HER inhibitor is selected from the group consisting of trastuzumab—an anti-ERBB2 antibody that binds ERBB2 domain IV; pertuzumab—an anti-ERBB2 antibody that binds ERBB2 domain II and prevents dimerization; anti-ERBB3.1—an anti-ERBB3 that blocks ligand binding (binds domain III); anti-ERBB3.2—an anti-ERBB3 antibody, that binds domain III and blocks ligand binding; MEHD7945A—a dual ERBB3/EGFR antibody that blocks ligand binding (binds domain III of EGFR and ERBB3); cetuximab—an EGFR antibody that blocks ligand binding (binds to domain III of EGFR); Lapatinib—a dual ERBB2/EGFR small molecule inhibitor; and GDC-094148—a PI3K inhibitor.

In another aspect, the present invention provides an anti-cancer therapeutic agent for use in a method of treating an ErbB3 cancer in a subject, said method comprising (i) detecting in a biological sample obtained from the subject the presence or absence of an amino acid mutation in a nucleic acid sequence encoding ErbB3, wherein the mutation results in an amino acid change at at least one position of the ErbB3 amino acid sequence (as described herein), wherein the presence of the mutation is indicative of the presence of cancer in the subject from which the sample was obtained; and (ii) if a mutation is detected in the nucleic acid sequence, administering to the subject an effective amount of the anti-cancer therapeutic agent.

Combination Therapy

It is contemplated that combination therapies may be employed in the methods. The combination therapy may include but are not limited to, administration of two or more cancer therapeutic agents. Administration of the therapeutic agents in combination typically is carried out over a defined time period (usually minutes, hours, days or weeks depending upon the combination selected). Combination therapy is intended to embrace administration of these therapeutic agents in a sequential manner, that is, wherein each therapeutic agent is administered at a different time, as well as administration of these therapeutic agents, or at least two of the therapeutic agents, in a substantially simultaneous manner.

The therapeutic agent can be administered by the same route or by different routes. For example, an ErbB antagonist in the combination may be administered by intravenous injection while a chemotherapeutic agent in the combination may be administered orally. Alternatively, for example, both of the therapeutic agents may be administered orally, or both therapeutic agents may be administered by intravenous injection, depending on the specific therapeutic agents. The sequence in which the therapeutic agents are administered also varies depending on the specific agents.

In one aspect, the present invention provides a method of treating an individual having an HER3/ErbB3 cancer identified by one or more of the somatic mutations described herein, wherein the method of treatment comprises administering more than one ErbB inhibitor. In one embodiment, the method comprises administering an ErbB3 inhibitor, e.g., an ErbB3 antagonist, and at least one additional ErbB inhibitor, e.g., an EGFR, an ErbB2, or an ErbB4 antagonist. In another embodiment, the method comprises administering an ErbB3 antagonist and an EGFR antagonist. In one other embodiment, the method comprises administering an ErbB3 antagonist and an ErbB2 antagonist. In yet another embodiment, the method comprises administering an ErbB3 antagonist and an ErbB4 antagonist. In some embodiments, at least one of the ErbB antagonists is an antibody. In another embodiment, each of the ErbB antagonists is an antibody.

Kits

For use in the applications described or suggested herein, kits or articles of manufacture are also provided. Such kits may comprise a carrier means being compartmentalized to receive in close confinement one or more container means such as vials, tubes, and the like, each of the container means comprising one of the separate elements to be used in the method. For example, one of the container means may comprise a probe that is or can be detectably labeled. Such probe may be a polynucleotide specific for a polynucleotide comprising an ErbB3 somatic mutation associated with cancer as disclosed herein. Where the kit utilizes nucleic acid hybridization to detect a target nucleic acid, the kit may also have containers containing nucleotide(s) for amplification of the target nucleic acid sequence and/or a container comprising a reporter means, such as a biotin-binding protein, such as avidin or streptavidin, bound to a reporter molecule, such as an enzymatic, florescent, or radioisotope label. In one embodiment, the kits of the present invention comprise one or more ErbB3 cancer detecting agents as described herein. In a preferred embodiment, the kit comprises one or more ErbB3 gastrointestinal cancer detecting agent, or one or more ErbB3 lung cancer detecting agent, as described herein. In another embodiment, the kit further comprises a therapeutic agent (e.g., an ErbB3 inhibitor), as described herein.

In other embodiments, the kit may comprise a labeled agent capable of detecting a polypeptide comprising an ErbB3 somatic mutation associated with cancer as disclosed herein. Such agent may be an antibody which binds the polypeptide. Such agent may be a peptide which binds the polypeptide. The kit may comprise, for example, a first antibody (e.g., attached to a solid support) which binds to a polypeptide comprising a genetic variant as disclosed herein; and, optionally, a second, different antibody which binds to either the polypeptide or the first antibody and is conjugated to a detectable label.

Kits will typically comprise the container described above and one or more other containers comprising materials desirable from a commercial and user standpoint, including buffers, diluents, filters, needles, syringes, and package inserts with instructions for use. A label may be present on the container to indicate that the composition is used for a specific therapy or non-therapeutic application, and may also indicate directions for either in vivo or in vitro use, such as those described above. Other optional components in the kit include one or more buffers (e.g., block buffer, wash buffer, substrate buffer, etc), other reagents such as substrate (e.g., chromogen) which is chemically altered by an enzymatic label, epitope retrieval solution, control samples (positive and/or negative controls), control slide(s) etc.

In another aspect, the present invention provides the use of an ErbB3 cancer detecting agent in the manufacture of a kit for detecting cancer in a subject. In one embodiment, the detection of an ErbB3 cancer comprises detecting in a biological sample obtained from the subject the presence or absence of an amino acid mutation in a nucleic acid sequence encoding ErbB3, wherein the mutation results in an amino acid change at at least one position of the ErbB3 amino acid sequence (as described herein), wherein the presence of the mutation is indicative of the presence of cancer in the subject from which the sample was obtained.

Methods of Marketing

The invention herein also encompasses a method for marketing the disclosed methods of diagnosis or prognosis of cancer comprising advertising to, instructing, and/or specifying to a target audience, the use of the disclosed methods.

Marketing is generally paid communication through a non-personal medium in which the sponsor is identified and the message is controlled. Marketing for purposes herein includes publicity, public relations, product placement, sponsorship, underwriting, and the like. This term also includes sponsored informational public notices appearing in any of the print communications media.

The marketing of the diagnostic method herein may be accomplished by any means. Examples of marketing media used to deliver these messages include television, radio, movies, magazines, newspapers, the internet, and billboards, including commercials, which are messages appearing in the broadcast media.

The type of marketing used will depend on many factors, for example, on the nature of the target audience to be reached, e.g., hospitals, insurance companies, clinics, doctors, nurses, and patients, as well as cost considerations and the relevant jurisdictional laws and regulations governing marketing of medicaments and diagnostics. The marketing may be individualized or customized based on user characterizations defined by service interaction and/or other data such as user demographics and geographical location.

The following examples are offered for illustrative purposes only, and are not intended to limit the scope of the present invention in any way.

All patent and literature references cited in the present specification are hereby incorporated by reference in their entirety.

### EXAMPLES

### Example

### Oncogenic ERBB3 Mutations in Human Cancers

Given the importance of ERBB3 in human cancers, we systematically surveyed human cancers and identified recurring somatic mutations and also show that these mutations are transforming. Further, we evaluated targeted therapeutics in ERBB3-mutant driven animal models of cancer and show that a majority of them are effective in blocking ERBB3-mutant driven oncogenesis.

### Materials and Methods

Tumor DNA, Mutation and Genomic Amplification

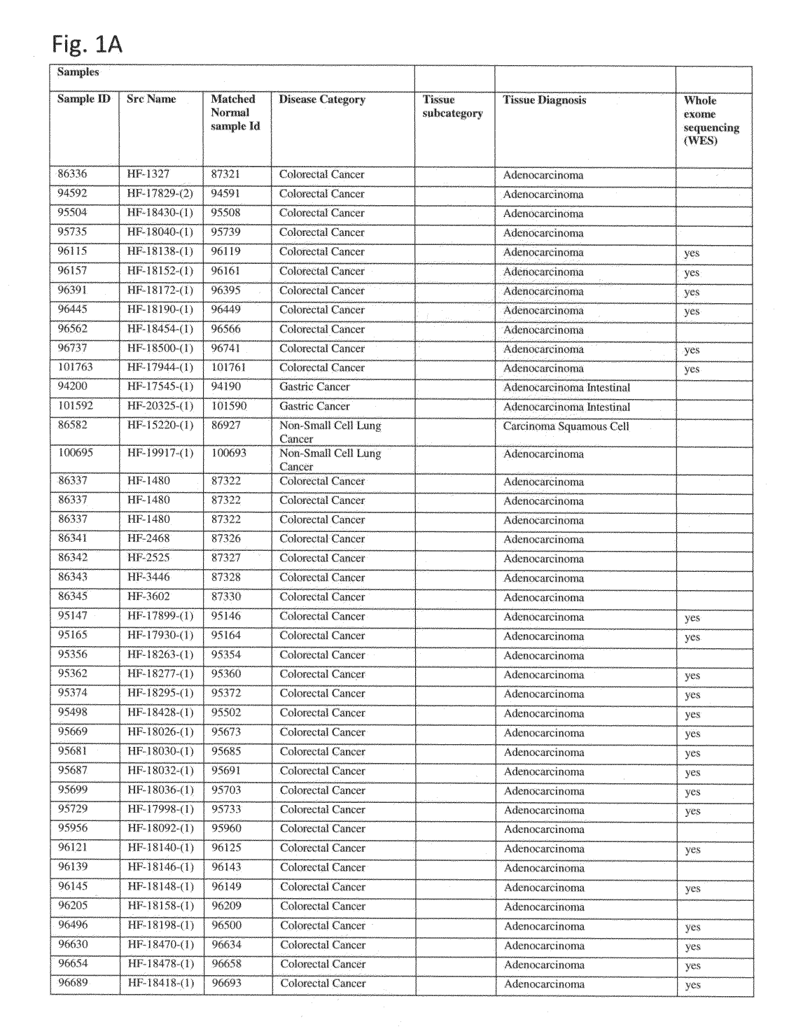

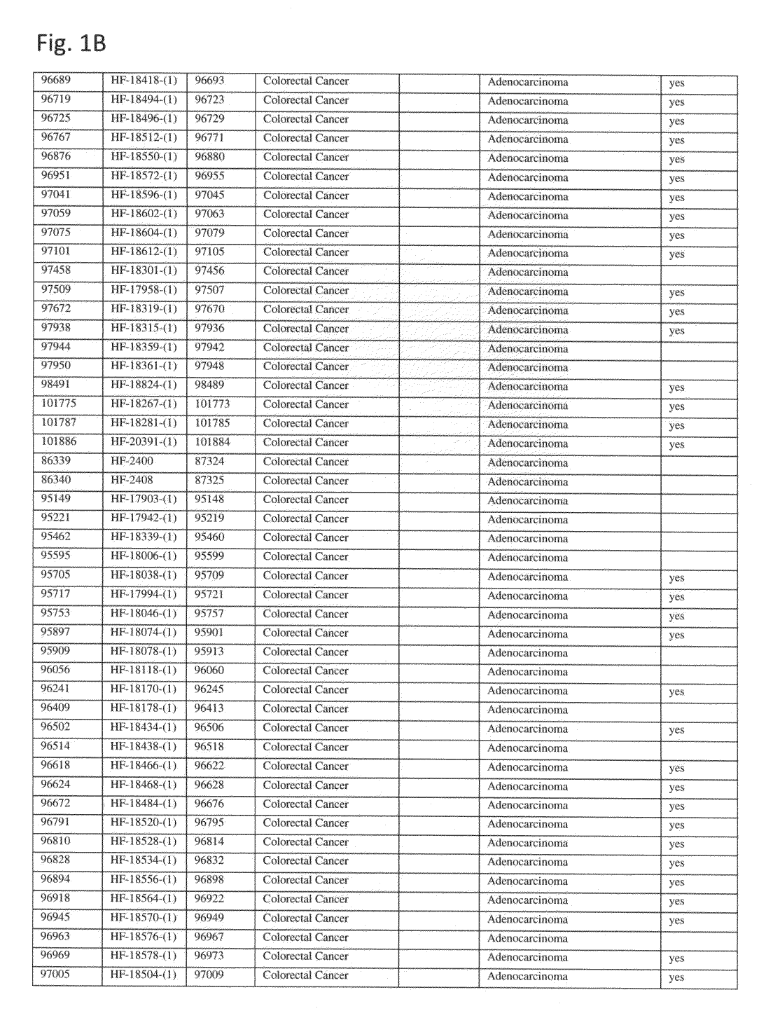

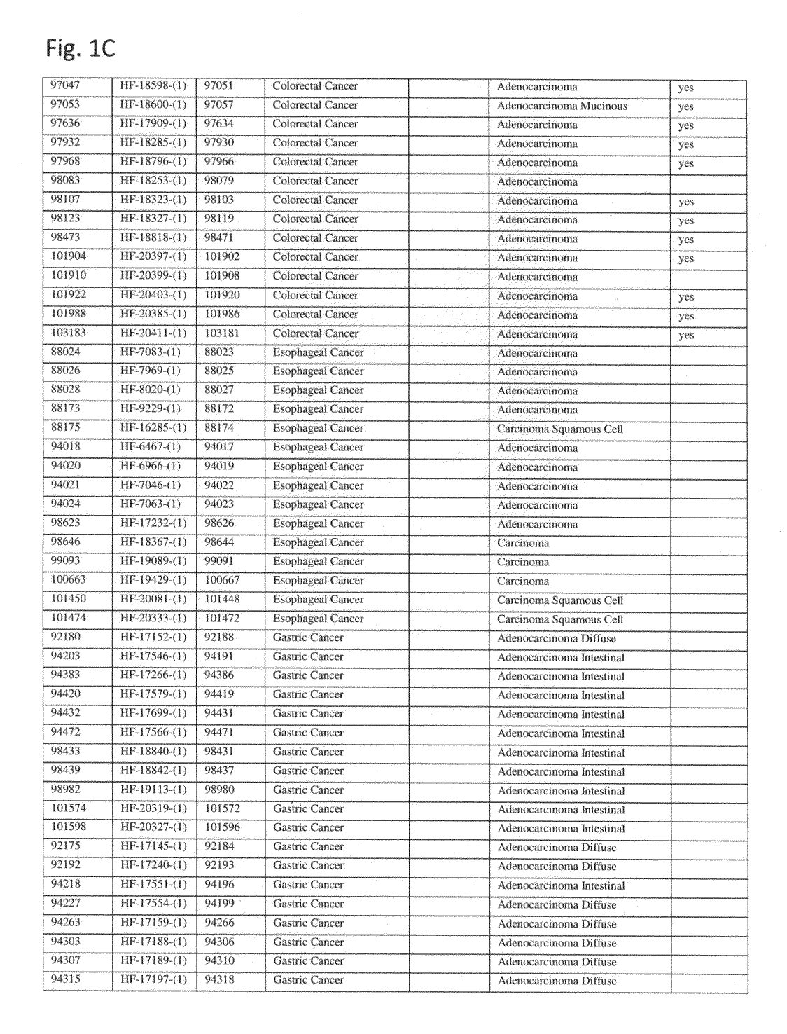

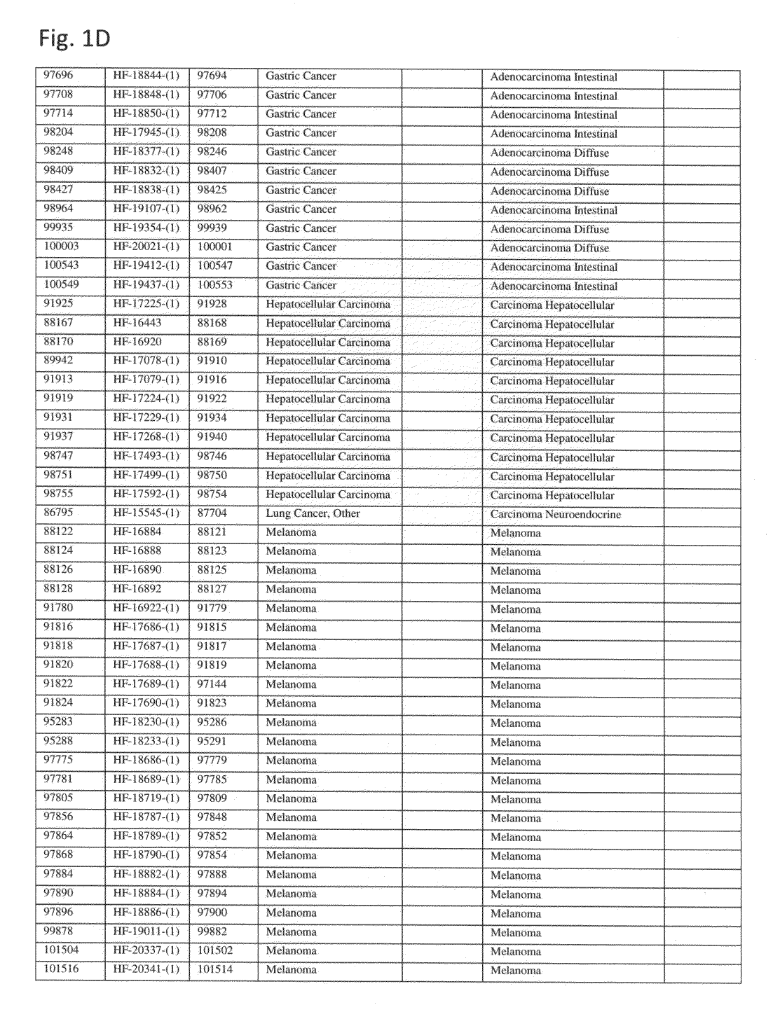

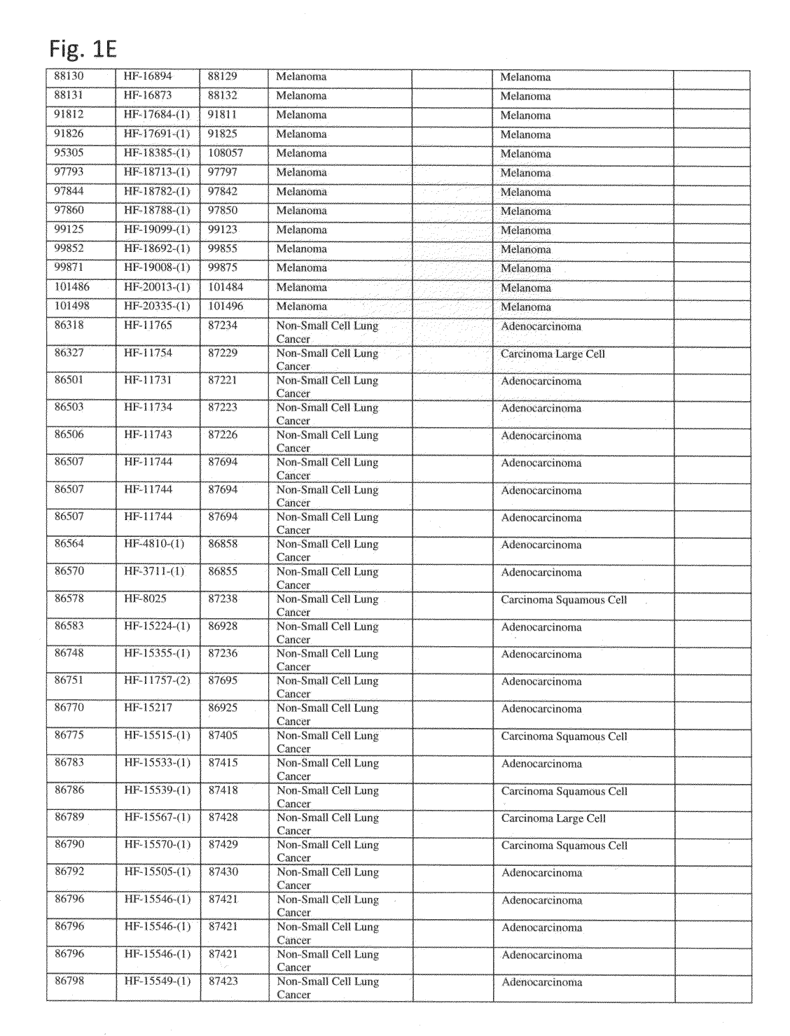

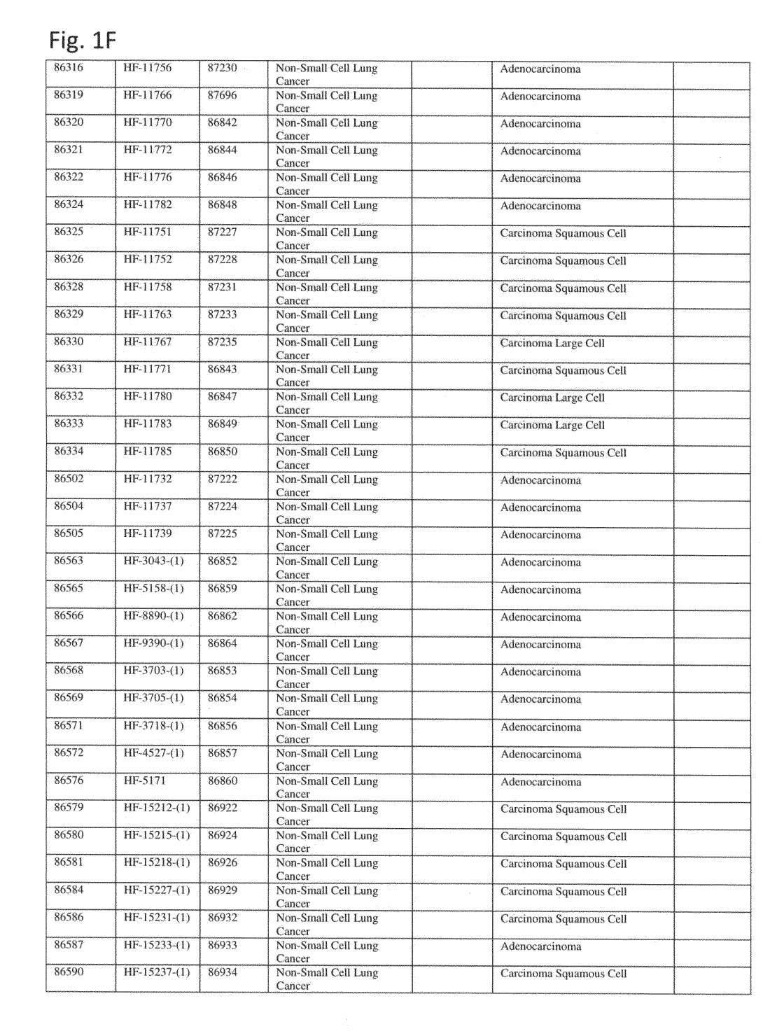

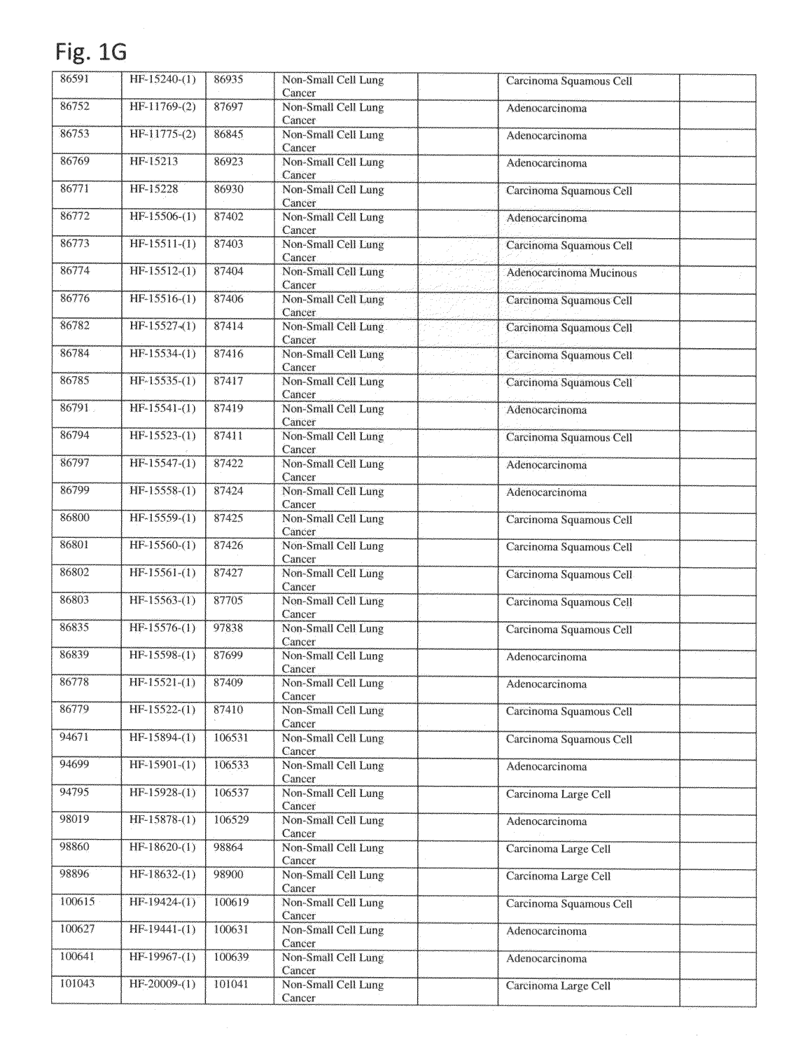

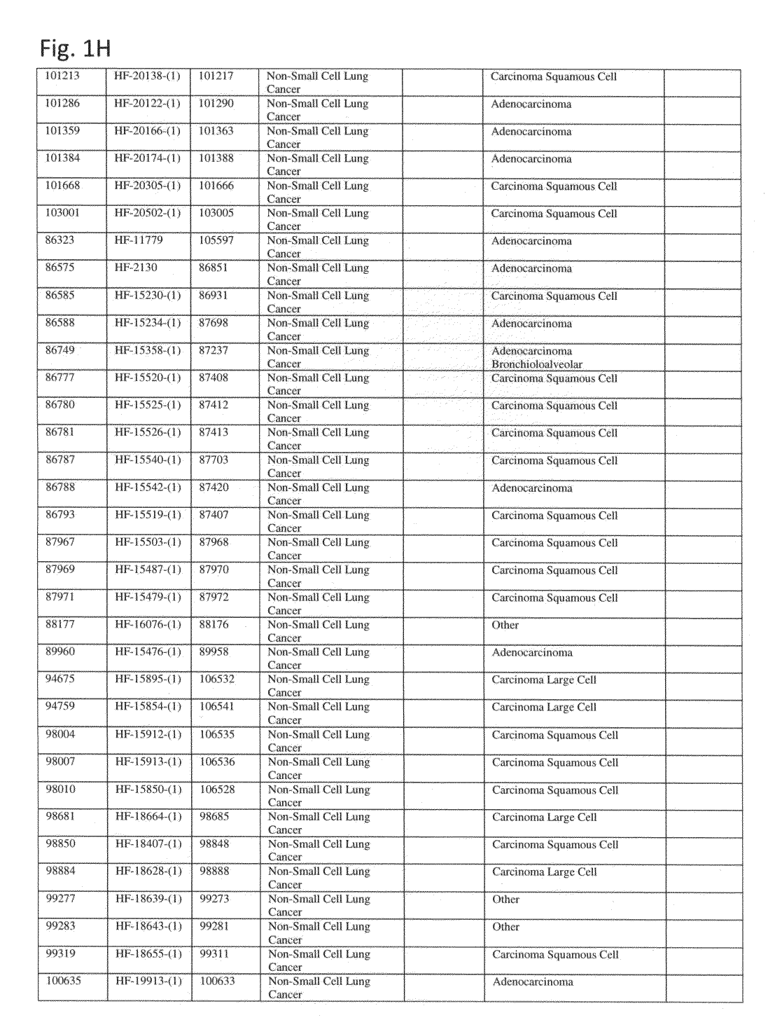

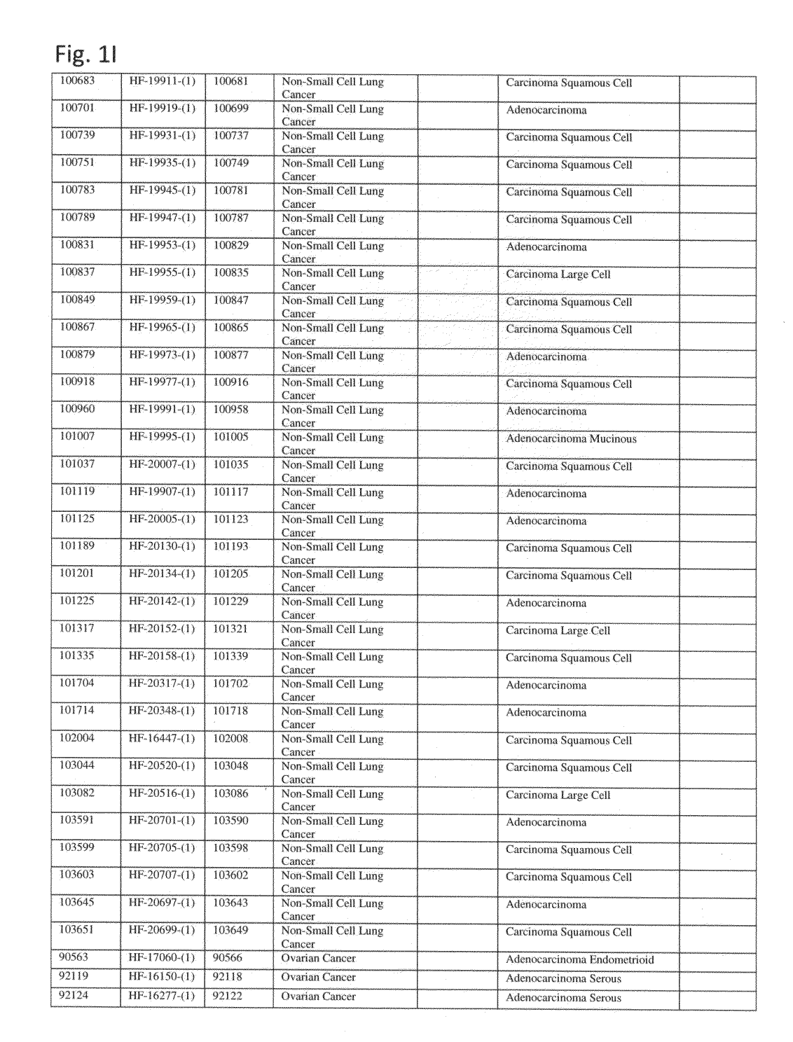

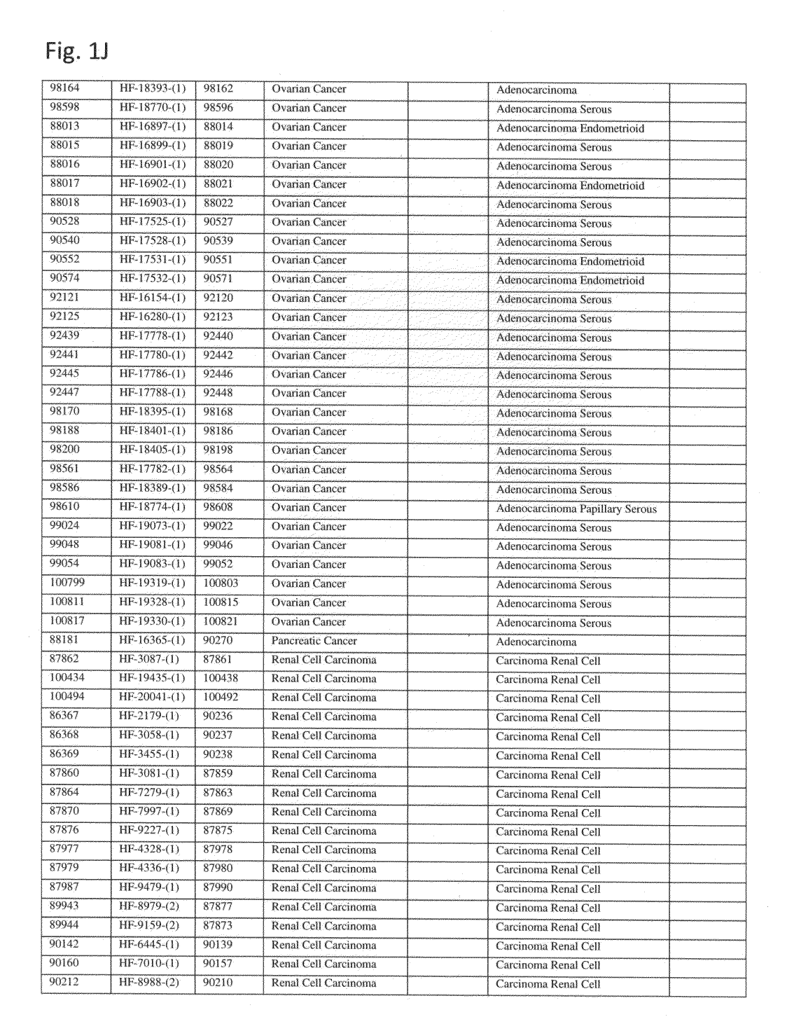

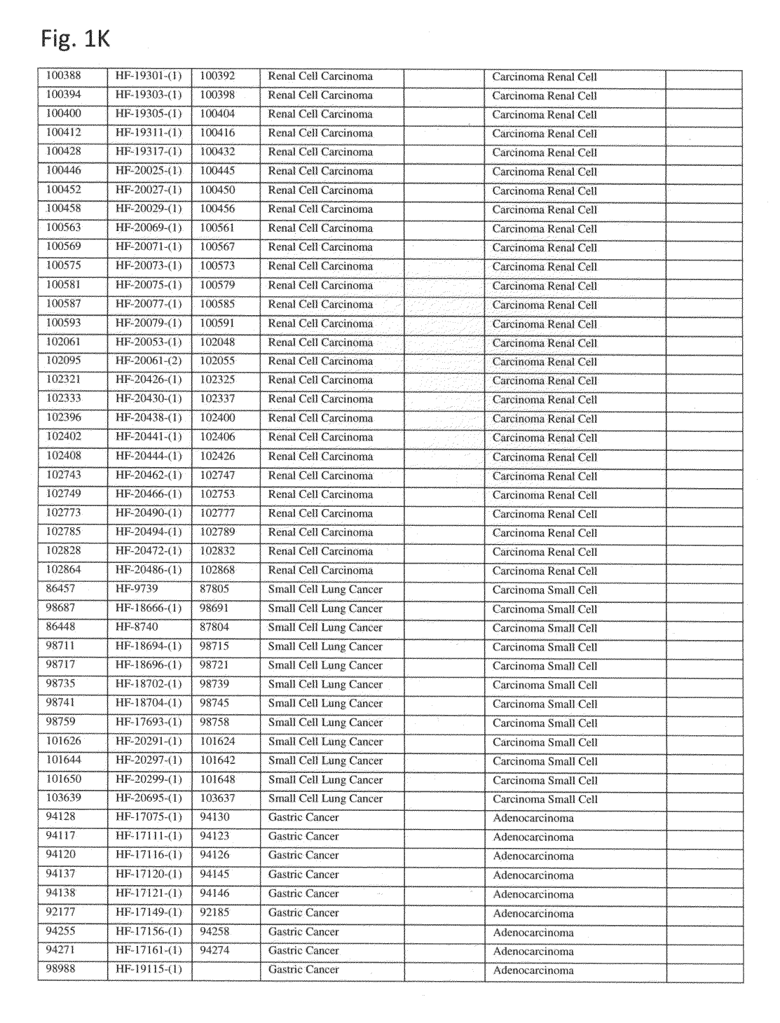

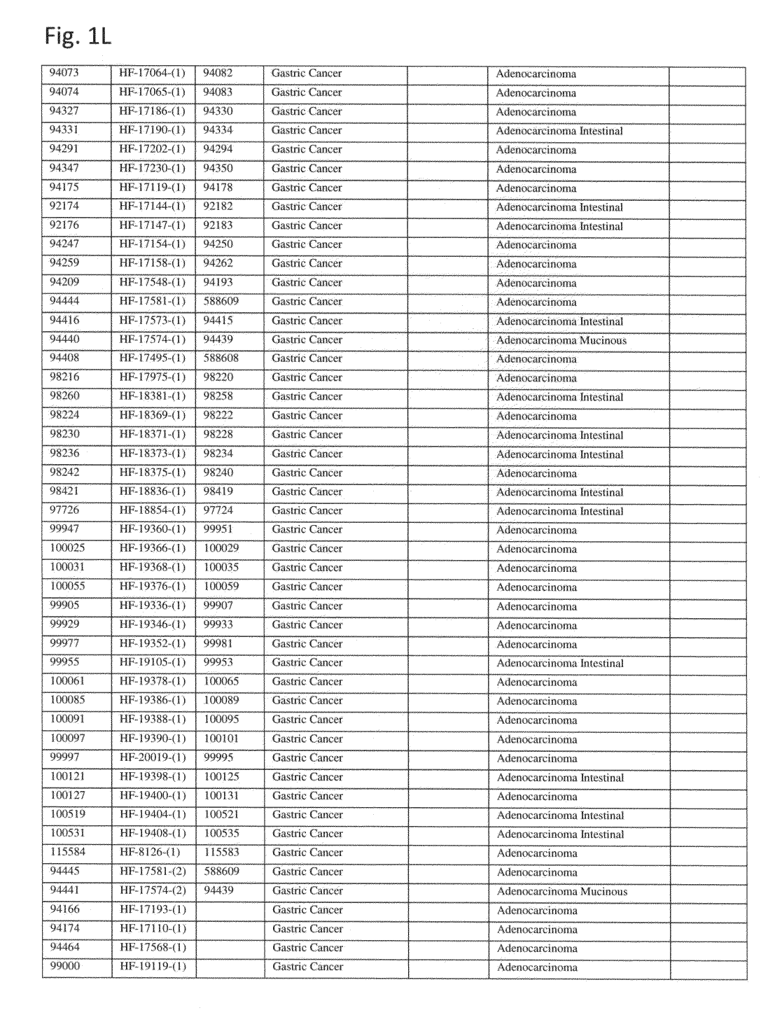

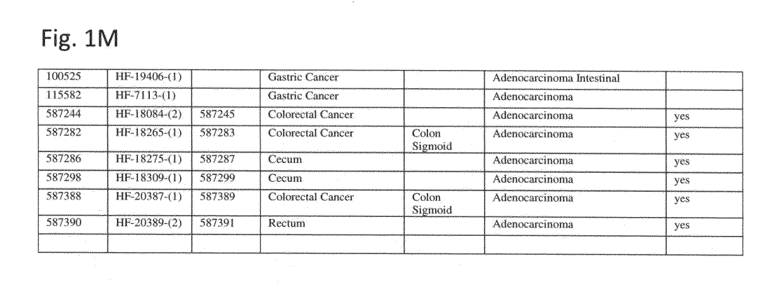

Appropriately consented primary human tumor samples were obtained from commercial sources (FIG. 1). The human tissue samples used in the study were de-identified (double-coded) prior to their use and hence, the study using these samples is not considered human subject research under the US Department of Human and Health Services regulations and related guidance (45 CFR Part 46). Tumor content in all the tumors used was confirmed to be >70% by pathology review. Tumor DNA was extracted using Qiagen Tissue easy kit. (Qiagen, CA). All coding exons of ERBB3 were amplified using primers listed in Table 1 below (Applied Biosystems, CA). The PCR products were generated using two pairs of primers, an outer pair and an inner pair to increase the specificity (Table 1), using standard PCR conditions were sequenced using 3730×1 ABI sequencer. The sequencing data was analyzed for presence of variants not present in the dbSNP database using Mutation Surveyor (Softgenetics, PA) and additional automated sequence alignment programs. The putative variants identified were confirmed by DNA sequencing or mass spectrometry analysis (Sequenom, CA) of the original tumor DNA followed by confirmation of its absence in the adjacent matched normal DNA by a similar process applied to the tumor DNA. Representative normal ERBB3 nucleic acid and amino acid sequences are provided in FIGS. 2 and 3, respectively.

TABLE 1
Primers used for PCR and sequencing

| ERBB3 | | | |
|---|---|---|---|
| exon | Target_ID | 5p Outer primer | 3p Outer Primer |
| 1 | DNA519201 | TCCCCTGCCATCC | CCCGAGCCTGACC |
| 2 | DNA519202 | GGCCACTACAGCTTC | TCCCAGATGACAGCC |
| 3 | DNA519203 | GCGTAACTCCGTCTCA | GGCCCTCTATTGCTTAG |
| 4 | DNA519204 | CTCCTCATCTTATAAAGGG | TGGTTTAGATTCCAGGAGA |
| 5 | DNA519205 | CGCCCCTTGTTGACA | CACTGAGGAGCACAGAT |
| 6 | DNA519206 | ATCAGAAGACTGCCAGA | TGTGGACAGCGAGGT |
| 7 | DNA519207 | CCAGTGCTGCCATGAT | GGAGGACTGGACGTA |
| 8 | DNA519208 | CAAATAGTGAAGAGACTTTTGAAT | ATCTTGGTGCAGTTCACAA |
| 9 | DNA519209 | CTGTCCTCCTGACAAGA | ATGGAGGATGTGTTAAGCA |
| 10 | DNA519210 | CTTGTTTGCACAAGATGCT | GACTGGATGTTCAGGTA |
| 11 | DNA519211 | TCACAGGTGAGTGGC | GATCCACTGAGAGGG |
| 12 | DNA519212 | CCTCAAAACCAAAGGGTTT | AGGACTCCCAGCAAG |
| 13 | DNA519213 | AGGGTCTGCTAGGTG | CCAAGTCCTGACCTTC |
| 14 | DNA519214 | CAGTCAAGGATGGGTG | TCCCAAGGTCAATTCCATA |
| 15 | DNA519215 | TGGAGCATCTGGGGA | CACCCACCTCGGC |
| 16 | DNA519216 | TCAAGGGAGTTTCACAGAA | CAGTCTTAGACTACTGAAAG |
| 17 | DNA517682 | CTTTCAGTAGTCTAAGACTG | ACCACACTACTTCCTTGA |
| 18 | DNA517683 | CAGGGTCTGTACCTC | TGCAGACTGGAATCTTGAT |
| 19 | DNA517684 | GAAGCTTAAAGTGCTTGG | GAAACCAACAGGTTCACA |
| 20 | DNA517685 | GGAGAGAGGACAATATTAG | CGCTCACATGCTCTG |
| 21 | DNA517686 | CCCAAAACCAACCCTC | CCAGTCCCAAGTTCTTG |
| 22 | DNA517687 | AGAGCGAGACTCCGT | CTGTCACACCTGTTGC |
| 23 | DNA517688 | GATGCCCTCTCTACC | CAGCCTGGGTGACAAT |
| 24 | DNA517689 | AGATGGGGTTTCACTATGT | CTCTACTTCCTCTAGCTT |
| 25 | DNA519217 | GCCCAACCTTTAAAGAAC | TGATGGACTTAAAAGGCTC |
| 26 | DNA519218 | GCCTACCAGTTGGAAC | CCTCAGGTGATCCACT |
| 27a | DNA519219_1 | GGCAGTGAACAACCCA | ATAACCGTTGACATCCTC |
| 27b | DNA519219_2 | CGTCCAGTCTCTCTACA | GAGGAGGGAGTACCT |
| 28a | DNA519220_1 | CTCAAAGGTGCCTGAC | CCCCTGAAAAGCTCTC |
| 28b | DNA519220_2 | CTTGAGGAGCTGGGTT | GTCAAAATGTTTAAAAGCCTCC |

| ERBB3 | | | Sequencing |
|---|---|---|---|
| exon | 5p Inner Primer (F) | 3p Inner Primer (F) | primers |
| 1 | CGCGGCCGTGACT | AATGCCGCCCTCG | F & R |
| 2 | AGAAGAGAGAAAGCTCTC | TACAACAGTGAGACCATAG | F & R |
| 3 | AGATCGCACTATTGTACTC | TAGCTCCCCCTACTG | F & R |
| 4 | CTGGACAGGTGACTGA | CTGCTCCTTTTCTTGAAACA | F & R |
| 5 | CTGGGTTGGGACTAG | GGCCCAAAGCAGTGA | F & R |
| 6 | TTGCAAGGGGCGATG | AGCTGGAAAGTTAGCTTG | F & R |
| 7 | TGTGCTCCTCAGTGTAA | GGTGATAGCTGAAGTCAT | F & R |
| 8 | CTTACTTCTGCTCCTTGTA | AAGTCCAGGTTGCCC | F & R |
| 9 | GATCAAACATCCTGTGTC | GATGTTCCTGAGGGGA | F & R |
| 10 | CCCTTAATTCTTTGAGTCTTG | ACACTGAAGTTGTGCATGT | F & R |
| 11 | GTCTTCCGGACAGTAC | GAAATTTGCTCAGTGCTAGT | F & R |
| 12 | CACTGTCTCATACAGCA | GGAGAGGAGTCTGAG | F & R |
| 13 | CAGAGACTGCGGTGA | TCCCTGTAGTGGGGA | F & R |
| 14 | CTTTCTGAATGGGTACAGTA | GTCAGGAAGAATCAGATC | F & R |
| 15 | GATCTCCAAGGGAGAC | TCTCGAACTCCCGAC | F & R |
| 16 | GAACCTGGAATAACCTCA | GACCAACCTAAATCTGG | F & R |
| 17 | GCTTCTGGACTTCCC | CCAGTGTTCTTCTAGGG | F & R |
| 18 | GCACAAATAACTTCCTCAGTT | CCGTCCACTCTTGTC | F & R |
| 19 | CTTCAAAGAGACAGAGCTAA | TAAGAGACACAAAAGGTATTATCT | F & R |
| 20 | AAGGAAATTCTGTATGCCG | CTTCACTCGCTTGCC | F & R |
| 21 | AAGGATCTAGGTTGTGC | GCGTGAGCCACCG | F & R |
| 22 | CACTGCACTCCAGTCT | CCGAAGGTCATCAACTC | F, R & R1 |
| 23 | CTGGAGCTATGGTCAGT | CCAAGATTGATTGCACC | F, F1 & R |
| 24 | AGATAGCTGGGACTTTAG | GTCTAGGTCTAGTTCTG | F & R |
| 25 | GTTGGATGATTGATGAGAAC | AAGATTACCCTGGTTCATG | F & R |
| 26 | CAACCACCACACTGG | ATTACAGGTGTGCACCA | F & R |
| 27a | GCGACAAGAACAAGACT | GTGTGTATCTGGCATGA | F, R & R2 |
| 27b | TGGGAGCAGTGAACG | CAGAACTGAGACCCAC | F & R |
| 28a | CATGCCAGATACACACC | GGCGGGCATAATGGA | F & R |
| 28b | ATCCCCCTAGGCCAA | TACATACCATAAGAATTTTGTGTC | F & R |

F1 = TCACTGGCCCCAGTT; R1 = GCAGGAAGACATGGACT; R2 = CTCTTCCTCTAACCCG

Table 1 discloses the “5p Outer Primer” sequences as SEQ ID NOS 3-32, the “3p Outer Primer” sequences as SEQ ID NOS 33-62, the “5p Inner Primer” sequences as SEQ ID NOS 63-92, the “3p Inner Primer” sequences as SEQ ID NOS 93-122, and the “F1,” “R1,” and “R2” sequences as SEQ ID NOS 123-125, all respectively, in order of appearance.

Cell Lines

The IL-3-dependent mouse pro-B cell line BaF3 and MCF10A, a mammary epithelial cell, was purchased from ATCC (American Type Culture Collection, Manassas, Va.). BaF3 cells were maintained in RPMI 1640 supplemented with 10% (v/v) fetal bovine serum (Thermo Fisher Scientific, IL), 2 mM L-glutamine, 100 U/ml penicillin, 100 mg/ml streptomycin (complete RPMI) and 2 ng/mL mouse IL-3. MCF10A cells were maintained in DMEM: F12 supplemented with 5% (v/v) horse serum, 0.5 ng/ml hydrocortisone, 100 ng/ml cholera toxin, 10 μg/ml insulin, 20 ng/ml EGF, 2 mM L-glutamine, 100 U/ml penicillin and 100 mg/ml streptomycin.

Plasmids and Antibodies

A retroviral vector, pRetro-IRES-GFP (Jaiswal, B. S. et al. Cancer Cell 16, 463-474 (2009)), was used to stably express c-terminal FLAG-tagged ERBB3 wildtype and mutants. ERBB3 mutants used in the study were generated using Quick Change Site-Directed Mutagenesis Kit (Stratagene, Calif.). Retroviral constructs that express full length ERBB2 with an herpes simplex signal sequence of glycoprotein D (gD) N-terminal tag or EGFR fused to gD coding sequence after removing the native secretion signal sequence, as done with ERBB2 previously, was expressed using pLPCX retroviral vector (Clontech, CA) (Schaefer et al. J Biol Chem 274, 859-866 (1999)).

Antibodies that recognize pERBB3 (Y1289), pEGFR (Y1068), pERBB2 (T1221/2), pAKT (Ser473), pMAPK, total MAPK and AKT (Cell Signaling Technology, MA), gD (Genentech Inc., CA), β-ACTIN and FLAG M2 (Sigma Life Science, MO) and HRP-conjugated secondary antibodies (Pierce Biotechnology, IL) for western blots were used in the study.

Generation of Stable Cell Lines

Retroviral constructs encoding wild type or mutants ERBB3-FLAG and gD-EGFR or gD ERBB2 were transfected into Pheonix amphoteric cells using Fugene 6 (Roche, Basal). The resulting virus was then transduced into either BaF3 or MCF10A cells. Top 10% of the either empty vector, wild type or ERBB3 mutant retrovirus infected cells based on the expression of retroviral IRES driven GFP was sterile sorted by flow cytometry and characterized for expression of proteins by western blot. To generate stable lines expressing ERBB3 mutants along with EGFR or ERBB2, FACS sorted ERBB3 wild type or mutants expressing cells were infected with either wild type EGFR or ERBB2 virus. Infected cells were then selected with 1 μg/ml puromycin for 7 days. Pools of these cells were then used in further studies.

Survival and Proliferation Assay

BaF3 cells stably expressing the wild-type and mutant ERBB3 alone or together with EGFR or ERBB2, were washed twice in PBS and plated in 3×96-well plates in replicates of eight in complete RPMI medium without IL3. As needed cells were then treated with different concentration of NRG1 and anti-NRG1 antibody or different ERBB antibodies, tyrosine kinase or PI3K small molecule inhibitors to test their effects on survival or cell proliferation, where relevant as depicted in the figures. Viable cells at 0 h and 120 h were determined using Cell Titer-Glo luminescence cell viability kit (Promega Corp., WI) and Synergy 2 (Biotek Instrument, CA) luminescence plate reader. All the cell number values were normalized against Oh values. In order to assess proliferation of MCF10A stably expressing ERBB3-WT or mutants were washed twice in PBS and 5000 cells plated in 96-well plates in replicates of eight in triplicates serum-free media and allowed to proliferate for 5 days. Cell numbers were measure at day 0 and day 5 using the luminescence cell viability kit. Data presented shows mean±SEM of survival at day 5 relative to day 0. Mean and statistical significance was determined using GraphPad V software (GraphPad, CA).

Immunoprecipitation and Western Blot

To assess the level of heterodimeric ERBB3-ERBB2 receptor complex expressed on the cell surface, we crossed linked the cell surface proteins using membrane-impermeable cross-linkers bis(sulfosuccinimidyl) suberate (BS3) (Thermo scientific, IL), prior to immunoprecipitation. BaF3 cells either with or without ligand (NRG1) treatment were washed twice in cold 50 mM HEPES pH 7.5 and 150 mM NaCl were treated with 1 mM BS3 in HEPES buffer for 60 min at 4° C. The cross-linking was stopped by washing the cells with twice with 50 mM Tris-Cl and 150 mM NaCl, pH 7.5. Cells were then lysed in lysis buffer I (50 mM TrisHCl pH 7.5, 150 mM NaCl, 1 mM EDTA, 1% Triton X-100). For immunoprecipitation, clarified lysated were incubated overnight at 4° C. with anti-FLAG-M2 antibody coupled beads (Sigma, Mo.). The FLAG beads were washed three times using the lysis buffer I. The immunoprecipitated proteins remaining on the beads were boiled in SDS-PAGE loading buffer, resolved on a 4-12% SDS-PAGE (Invitrogen, CA) and transferred onto a nitrocellulose membrane. Immunoprecipitated proteins or proteins from lysates were detected using appropriate primary, HRP-conjugated secondary antibody and chemiluminescences Super signal West Dura chemiluminescence detection substrate (Thermo Fisher Scientific, IL).

For western blot studies MCF10A cells were serum starved and grown in the absence of EGF or NRG1. Similarly, status of ERBB receptors and downstream signaling components were assessed in BaF3 cells grown in the absence of IL-3.

Proximity Ligation Assay

BaF3 cell lines stably expressing wild type or P262H, G284R and Q809R ERBB3 mutants along with ERBB2 were grown to subconfluency. Cells were washed twice with PBS and incubated overnight in IL3-free RPMI medium. Cytospin preparations of these cells were made, air dried and fixed with 4% paraformaldehyde for 15 min and then permeabilized with 0.05% Triton in PBS for 10 min. After blocking for 60 min with Duolink blocking solution (Soderberg et al. Nat Methods 3, 995-1000 (2006)), cells were either incubated with anti-FLAG (rabbit) and anti-gD (mouse) or anti-ERBB3 (mouse) (Labvision, CA) and anti-ERBB2 (rabbit) (Dako, Denmark) antibodies for 1 hrs at room temperature. Duolink staining were performed using Duolink anti-rabbit plus and anti-mouse minus PLA probes and Duolink II detection reagents (Uppsala, Sweden) far red following manufacturer protocols (Soderberg et al. Nat Methods 3, 995-1000 (2006)). Image acquisition was done using Axioplan2, Zeiss microscope and appropriate filter for DAPI and Texas red at 63× objective. For quantitative measurement of signal, tiff image files were analyzed with Duolink image tool software after applying user-defined threshold.

Colony Formation Assay

BaF3 cells stably expressing EGFR (2×10⁵) or ERBB2 (50,000) along with ERBB3 wild-type or mutants, was mixed with 2 mls of IL3-free Methylcellulose (STEMCELL Technologies, Canada) and plated on to 6 well plates and when indicated, cells were treated with different ERBB antibodies or tyrosine kinase or PI3K small molecule inhibitors before plating. Plates were then incubated at 37° C. for 2 weeks. For MCF10A colony formation, 20,000 MCF10A cells stably expressing ERBB3-WT or mutants alone or in combination with EGFR or ERBB2 were mixed with 0.35% agar in DMEM: F12 lacking serum, EGF, and NRG1 and plated on 0.5% base agar. Plates were then incubated at 37° C. for 3 weeks. The presence of colonies was assessed using Gel count imager (Oxford Optronix Ltd, UK). The number of colonies in each plate was quantified using Gel count software (Oxford Optronix Ltd, UK).

Three-Dimensional Morphogenesis or Acini Formation Assay

MCF10A cells stably expressing ERBB3 wild type or mutants either alone or in combination of either EGFR or ERBB2 were seeded on growth factor reduced Matrigel (BD Biosciences, CA) in 8-well chamber slides following the protocol described previously (Debnath et al. Methods 30, 256-268 (2003)). Morphogenesis of acini was photographed on day 12-15 using zeiss microscope using 10× objective.

Complete extraction, fixation and immunostaining of day 13 3D cultures was performed as previously described (Lee et al. Nat Methods 4, 359-365 (2007)). Briefly, after extraction, the acini were fixed with methanol-acetone (1;1) and stained with rat anti-α6 integrin (Millipore, Billerica Mass.), rabbit anti Ki67 (Vector Labs, Burlingame, Calif.) and DAPI. Goat anti-rat Alexa Fluor 647 (Invitrogen, CA) and goat anti-rabbit Alexa Fluor 532 (Invitrogen, CA) secondary antibodies were used in the study. Confocal imaging was performed with a 40× oil immersion objective, using a Leica SPE confocal microscope.

Transwell Migration Study

MCF-10A cells stably expressing empty vector, wildtype ERBB3 or various mutants of ERBB3 (50,000 cells) were seeded on to 8 μm transwell migration chambers (Corning, #3422). The cells were allowed to migrate for 20 h in serum-free assay medium. Cells on the upper part of the membrane were scraped using a cotton swab and the migrated cells were fixed in 3.7% (v/v) paraformaldehyde and stained with 0.1% Crystal Violet. From every transwell, images were taken from five different fields under a phase contrast microscope at 20× magnification and the number of migrated cells was counted. The numbers obtained were also verified by staining the nuclei by Hoechst dye. The fold increase in migration observed in ERBB3 mutant expressing cells in comparison to the wild type ERBB3 expressing cells was calculated and Student t-test was performed to test for the significance with prism pad software.

Animal Studies

BaF3 cells (2×10⁶) expressing the ERBB3 wild-type or mutants along with ERBB2 were implanted into 8-12 week old Balb/C nude mice by tail vein injection. For in vivo antibody efficacy study, mice were treated with 40 mg/kg QW anti-Ragweed (control), 10 mg/kg QW trastuzumab, 50 mg/kg QW anti-ERBB3.1 and 100 mg/kg QW anti-ERBB3.2 starting on day 4 after cell implant. A total of 13 animals per treatment were injected. Of this 10 mice were followed for survival and 3 were used for necropsy at day 20 to assess disease progression by histological analysis of bone marrow, spleen and liver. Bone marrow and spleen single cell suspension obtained from these animals was also analyzed for the presence and proportion of GFP positive BaF3 cells by FACS analysis. When possible dead or moribund animals in the survival study were dissected to confirm the cause of death. Morphologic and histological analyses of spleen, liver and bone marrow was also done on these animals. Bone marrow, spleen and liver were fixed in 10% neutral buffered formalin, then processed in an automated tissue processor (TissueTek, CA) and embedded in paraffin. Four-micron thick sections were stained with H&E (Sigma, Mo.), and analyzed histologically for presence of infiltrating tumor cells. Photographs of histology were taken on a Nikon 80i compound microscope with a Nikon DS-R camera. All animal studies were performed under Genentech's Institutional Animal Care and Use Committee (IACUC) approved protocols.

Statistical Analyses

Error bars where presented represent mean±SEM. Student's t-test (two tailed) was used for statistical analyses to compare treatment groups using GraphPad Prism 5.00 (GraphPad Software, San Diego, Calif.). A P-value<0.05 was considered statistically significant (*p<0.05, **p<0.01, ***p<0.001 and ****p<0.0001). For Kaplan-Meier Method of survival analysis, log-rank statistics were used to test for difference in survival.

### Results

Identification of ERBB3 Mutations

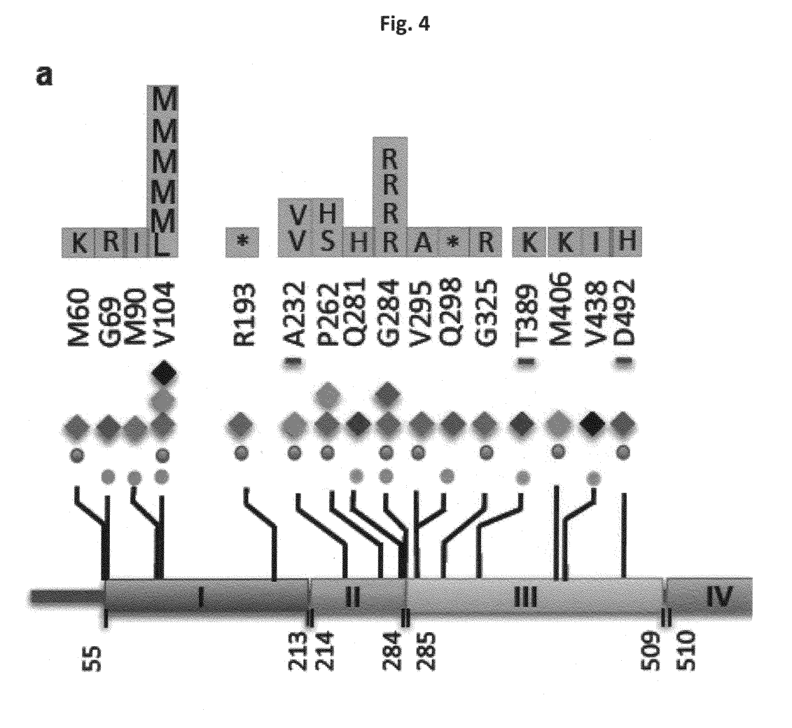

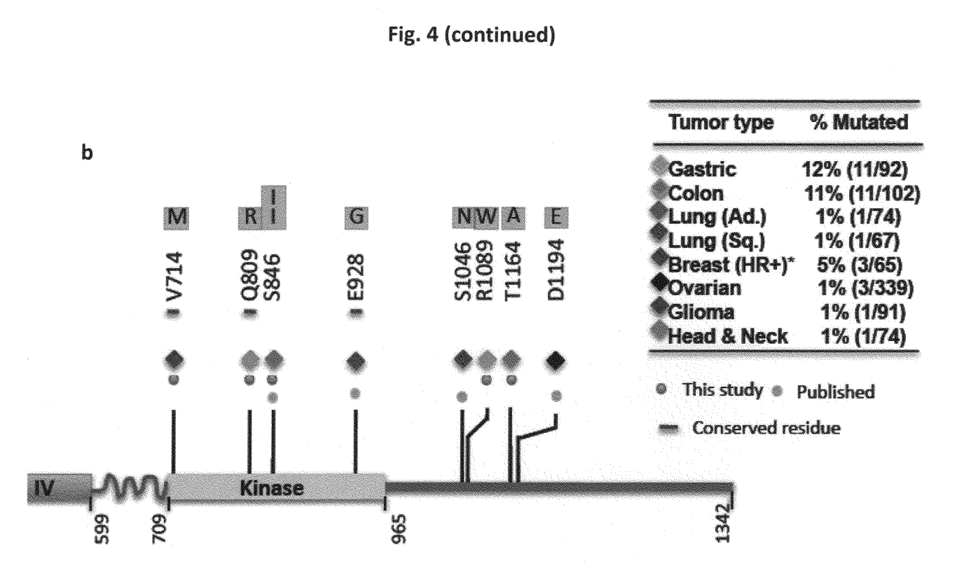

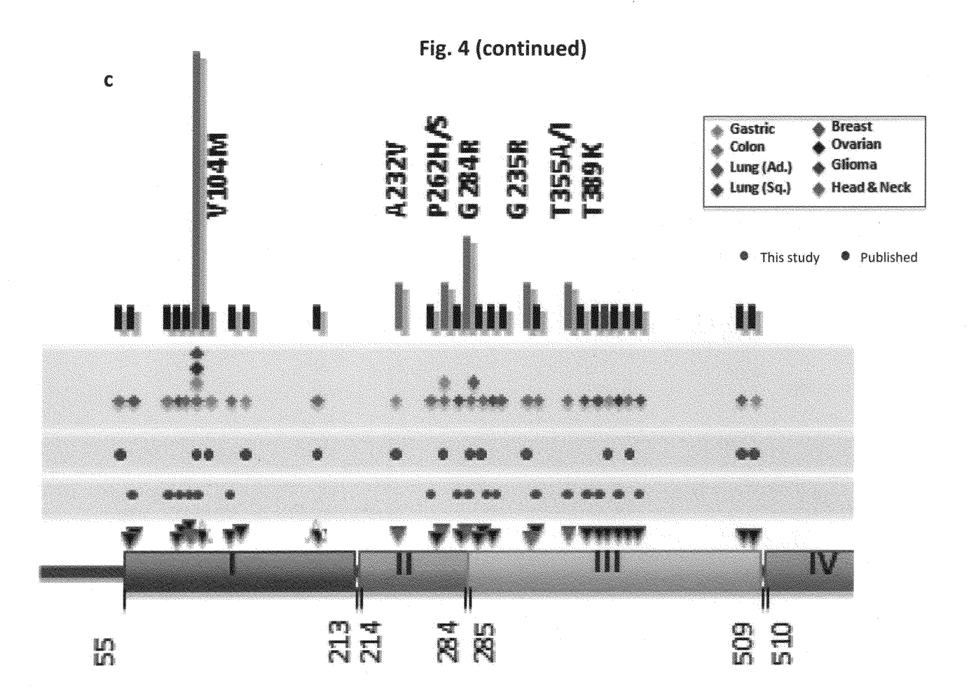

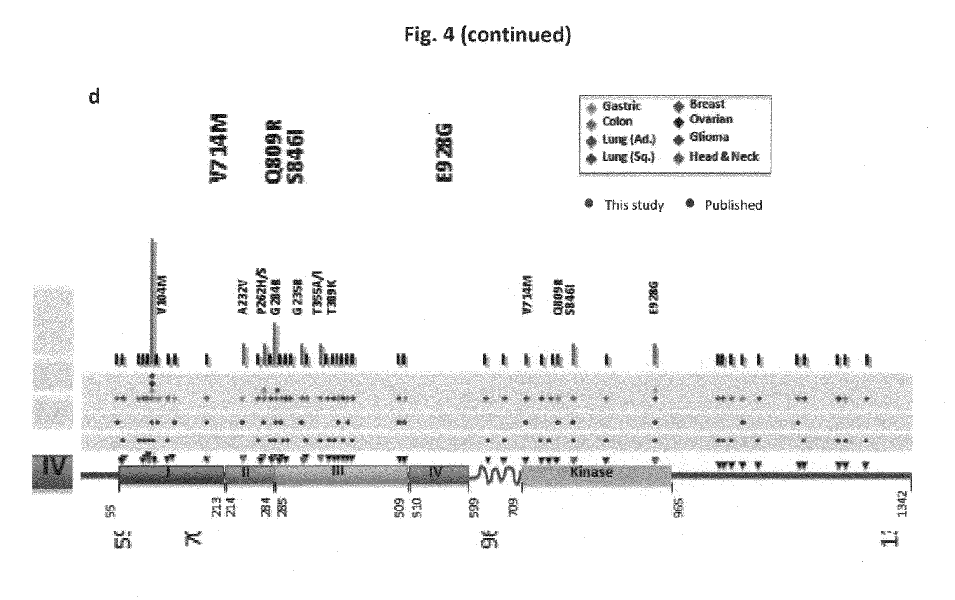

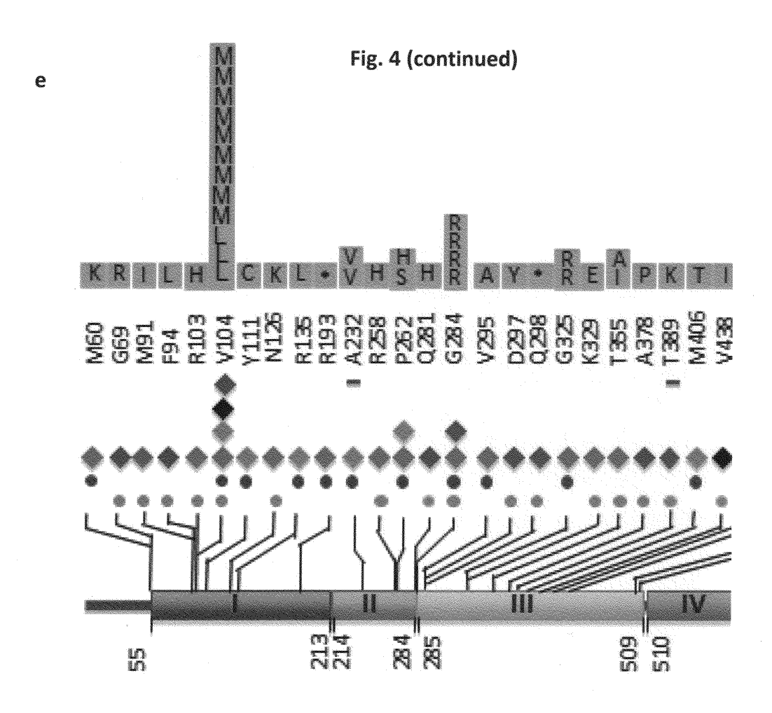

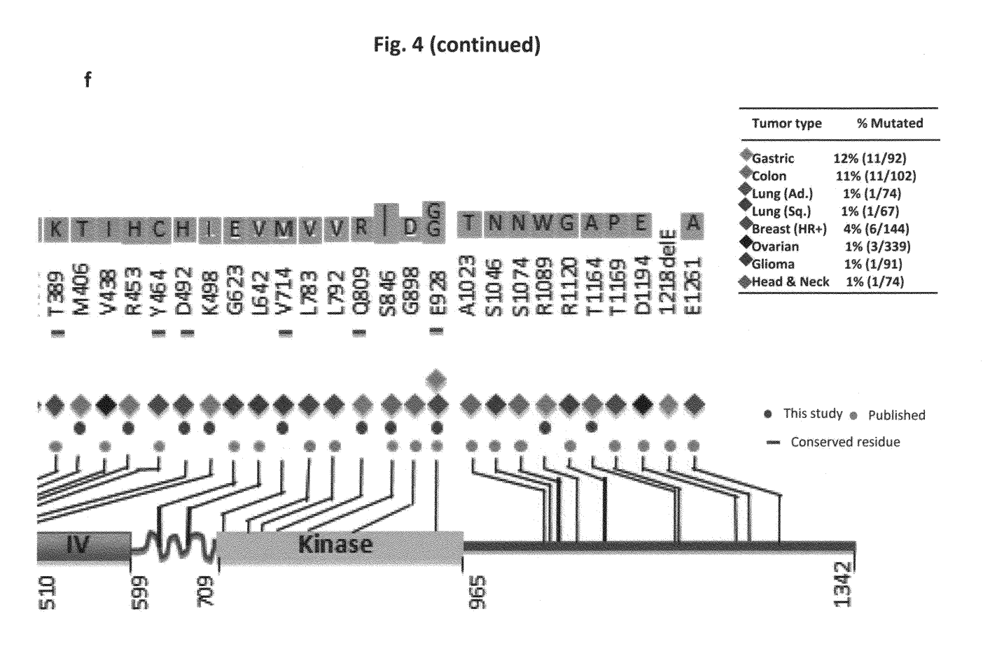

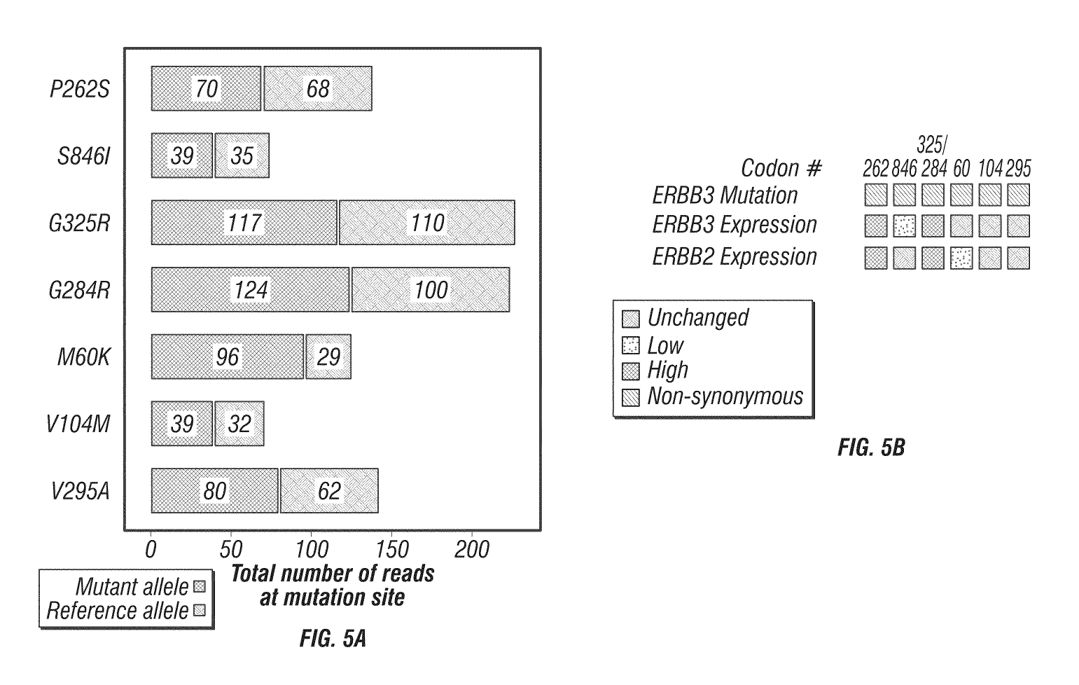

In performing whole exome sequencing of seventy primary colon tumors along with their matched normal samples, we identified somatic mutations in ERBB3 (Seshagiri, S. et al. Comprehensive analysis of colon cancer genomes identifies recurrent mutations and R-spondin fusions. (Mansuscript in Preparation 2011)). To further understand the prevalence of ERBB3 mutation in human solid tumors, we sequenced coding exons of ERBB3 in a total of 512 human primary tumor samples consisting of 102 (70 samples from the whole exome screen (Seshagiri, S. et al. Comprehensive analysis of colon cancer genomes identifies recurrent mutations and R-spondin fusions. (Mansuscript in Preparation 2011)) and 32 additional colon samples) colorectal, 92 gastric, 74 non-small-cell lung (NSCLC) adenocarinoma (adeno), 67 NSCLC (Squamous carcinoma), 45 renal carcinoma, 37 melanoma, 32 ovarian, 16 lung large cell, 15 esophageal, 12 small-cell lung cancer (SCLC), 11 hepatocellular (HCC), and 9 other cancers [4 lung cancer (other), 2 cecum, 1 lung (neuroendocrine), 1 pancreatic and 1 rectal cancer] (FIG. 1). We found protein altering ERBB3 mutations in 12% of gastric (11/92), 11% of colon (11/102), 1% of NSCLC (adeno; 1/74) and 1% of NSCLC (squamous; 1/67) cancers (FIG. 4). Though previous studies report sporadic protein altering ERBB3 mutations in NSCLC (squamous; 0.5% [3/188]), glioblastoma (1% [1/91]), hormone positive breast cancer (5% [3/65]), colon (1% [1/100]), ovarian cancer (1% [3/339]), and head and neck cancer (1%[1/74]), none have reported recurrent mutations nor have evaluated the functional relevance of these mutation in cancer (FIG. 4, and Tables 2 and 3). We confirmed all the mutations reported in this study to be somatic by testing for their presence in the original tumor DNA and absence in the matched adjacent normal tissue through additional sequencing and/or mass spectrometric analysis. Besides the missense mutations, we also found three synonymous (non-protein altering) mutations, one each in colon, gastric and ovarian cancers. Further, in colon tumors, using RNA-seq data (Seshagiri, S. et al. Comprehensive analysis of colon cancer genomes identifies recurrent mutations and R-spondin fusions. (Mansuscript in Preparation 2011)), we confirmed the expression of the ERBB3 mutants and the expression of ERBB2 in these samples (FIG. 5).

A majority of the mutations clustered mainly in the ECD region although some mapped to the kinase domain and the intracellular tail of ERBB3. Interestingly, among the ECD mutants were four positions, V104, A232, P262 and G284, that contained recurrent substitutions across multiple samples, indicating that these are mutational hotspots. Two of the four ECD hotspot positions identified in our analysis, V104 and G284, were previously reported mutated in an ovarian and a lung (adenocarinoma) sample respectively (Greenman et al. Nature 446, 153-158 (2007); Ding et al. Nature 455, 1069-1075 (2008)). Furthermore, most of the recurrent missense substitutions at each of the hotspot positions resulted in the same amino acid change indicative of a potential driver role for these mutations. We also identified a hotspot mutation, S846I, in the kinase domain when we combined our data with a single ERBB3 mutation previously published in colon cancer (Jeong et al. International Journal of Cancer 119, 2986-2987 (2006)).

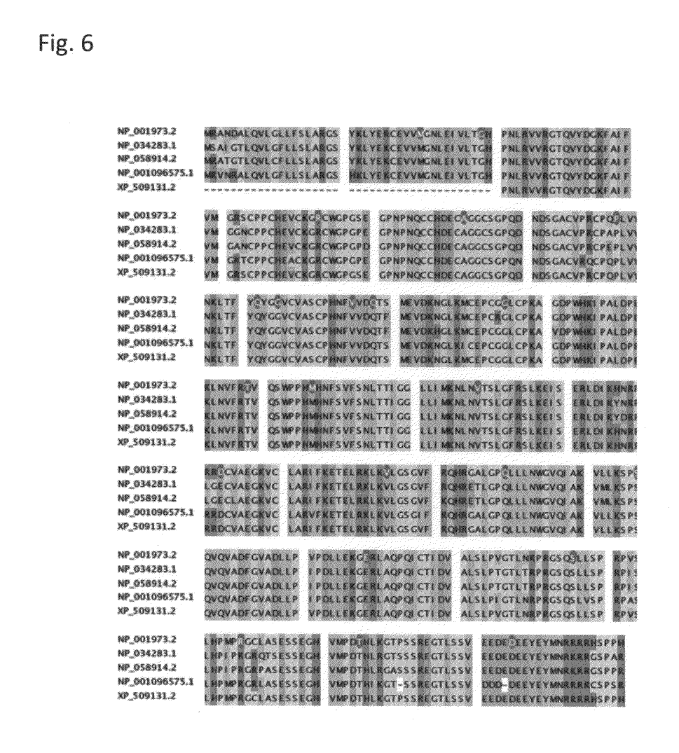

It is interesting to note that a majority of the mutated residues identified were conserved across ERBB3 orthologs (shown in FIG. 6, as well as the C. lupus (XP_{—}538226.2) sequence of SEQ ID NO:) and some of the residues were conserved between ERBB family members, which further suggest that these mutations likely have a functional effect.

TABLE 2
ERBB3 somatic mutations

| ENTREZ_GENE_ID | HUGO_GENE_SYMBOL | MUT_TYPE | MUT_EFFECT | MUT_LOCATION | CHROMOSOME | STRAND |
|---|---|---|---|---|---|---|
| 2065 | ERBB3 | Substitution | Nonsynonymous | Coding | 12 | + |
| 2065 | ERBB3 | Substitution | Nonsynonymous | Coding | 12 | + |
| 2065 | ERBB3 | Substitution | Nonsynonymous | Coding | 12 | + |
| 2065 | ERBB3 | Substitution | Nonsynonymous | Coding | 12 | + |
| 2065 | ERBB3 | Substitution | Nonsense | Coding | 12 | + |
| 2065 | ERBB3 | Substitution | Nonsynonymous | Coding | 12 | + |
| 2065 | ERBB3 | Substitution | Nonsynonymous | Coding | 12 | + |
| 2065 | ERBB3 | Substitution | Nonsynonymous | Coding | 12 | + |
| 2065 | ERBB3 | Substitution | Nonsynonymous | Coding | 12 | + |
| 2065 | ERBB3 | Substitution | Nonsynonymous | Coding | 12 | + |
| 2065 | ERBB3 | Substitution | Nonsynonymous | Coding | 12 | + |
| 2065 | ERBB3 | Substitution | Nonsynonymous | Coding | 12 | + |
| 2065 | ERBB3 | Substitution | Nonsynonymous | Coding | 12 | + |
| 2065 | ERBB3 | Substitution | Nonsynonymous | Coding | 12 | + |
| 2065 | ERBB3 | Substitution | Synonymous | Coding | 12 | + |
| 2065 | ERBB3 | Substitution | Nonsynonymous | Coding | 12 | + |
| 2065 | ERBB3 | Substitution | Synonymous | Coding | 12 | + |
| 2065 | ERBB3 | Substitution | Nonsynonymous | Coding | 12 | + |
| 2065 | ERBB3 | Substitution | Nonsynonymous | Coding | 12 | + |
| 2065 | ERBB3 | Substitution | Nonsynonymous | Coding | 12 | + |
| 2065 | ERBB3 | Substitution | Nonsynonymous | Coding | 12 | + |
| 2065 | ERBB3 | Substitution | Synonymous | Coding | 12 | + |
| 2065 | ERBB3 | Substitution | Nonsynonymous | Coding | 12 | + |
| 2065 | ERBB3 | Substitution | Nonsynonymous | Coding | 12 | + |
| 2065 | ERBB3 | Substitution | Nonsynonymous | Coding | 12 | + |
| 2065 | ERBB3 | Substitution | Nonsynonymous | Coding | 12 | + |
| 2065 | ERBB3 | Substitution | Nonsynonymous | Coding | 12 | + |
| 2065 | ERBB3 | Substitution | Nonsynonymous | Coding | 12 | + |
| 2065 | ERBB3 | Substitution | Nonsynonymous | Coding | 12 | + |
| 2065 | ERBB3 | Substitution | Nonsynonymous | Coding | 12 | + |
| 2065 | ERBB3 | Substitution | Nonsynonymous | Coding | 12 | + |

| ENTREZ_GENE_ID | GENOME_NT_POSITION_FROM* | GENOME_NT_POSITION_TO* | REFSEQ_TRANSCIPT_ID | NT_CHGE |
|---|---|---|---|---|
| 2065 | 56477631 | 56477631 | NM_001982.2 | 372T > A |
| 2065 | 56478854 | 56478854 | NM_001982.2 | 503G > T |
| 2065 | 56478854 | 56478854 | NM_001982.2 | 503G > A |
| 2065 | 56478854 | 56478854 | NM_001982.2 | 503G > A |
| 2065 | 56481390 | 56481390 | NM_001982.2 | 770C > T |
| 2065 | 56481660 | 56481660 | NM_001982.2 | 888C > T |
| 2065 | 56481856 | 56481856 | NM_001982.2 | 977C > T |
| 2065 | 56481857 | 56481857 | NM_001982.2 | 978C > A |
| 2065 | 56481922 | 56481922 | NM_001982.2 | 1043G > A |
| 2065 | 56481922 | 56481922 | NM_001982.2 | 1043G > A |
| 2065 | 56481922 | 56481922 | NM_001982.2 | 1043G > A |
| 2065 | 56482336 | 56482336 | NM_001982.2 | 1077T > C |
| 2065 | 56482425 | 56482425 | NM_001982.2 | 1166G > A |
| 2065 | 56482425 | 56482425 | NM_001982.2 | 1166G > A |
| 2065 | 56487150 | 56487150 | NM_001982.2 | 1489C > T |
| 2065 | 56487328 | 56487328 | NM_001982.2 | 1667G > C |
| 2065 | 56487675 | 56487675 | NM_001982.2 | 1801G > A |
| 2065 | 56490371 | 56490371 | NM_001982.2 | 2333G > A |
| 2065 | 56490980 | 56490980 | NM_001982.2 | 2619A > G |
| 2065 | 56491645 | 56491645 | NM_001982.2 | 2730G > T |
| 2065 | 56495133 | 56495133 | NM_001982.2 | 3683A > G |
| 2065 | 56495713 | 56495713 | NM_001982.2 | 4096G > A |
| 2065 | 56478854 | 56478854 | NM_001982.2 | 503G > A |
| 2065 | 56478854 | 56478854 | NM_001982.2 | 503G > A |
| 2065 | 56478876 | 56478876 | NM_001982.2 | 525A > G |
| 2065 | 56478948 | 56478948 | NM_001982.2 | 597G > T |
| 2065 | 56481660 | 56481660 | NM_001982.2 | 888C > T |
| 2065 | 56486803 | 56486803 | NM_001982.2 | 1410T > C |
| 2065 | 56487212 | 56487212 | NM_001982.2 | 1551G > A |
| 2065 | 56487560 | 56487560 | NM_001982.2 | 1686A > T |
| 2065 | 56494908 | 56494908 | NM_001982.2 | 3458C > T |

| ENTREZ_GENE_ID | AA_CHGE | PROTEIN_DOMAIN | COSMIC_IDS | SAMPLE_ID | DISEASE_CATEGORY |
|---|---|---|---|---|---|
| 2065 | 60M > K | Recep_L_domain\|PF01030.15 | | 96391 | Colorectal Cancer |
| 2065 | 104V > L | Recep_L_domain\|PF01030.15 | | 86336 | Colorectal Cancer |
| 2065 | 104V > M | Recep_L_domain\|PF01030.15 | 20710 | 96445 | Colorectal Cancer |
| 2065 | 104V > M | Recep_L_domain\|PF01030.15 | 20710 | 95735 | Colorectal Cancer |
| 2065 | 193R > O | Furin-like\|PF00757.11 | | 95735 | Colorectal Cancer |
| 2065 | 232A > V | Furin-like\|PF00757.11 | | 94200 | Gastric Cancer |
| 2065 | 262P > S | Furin-like\|PF00757.11 | | 96157 | Colorectal Cancer |
| 2065 | 262P > H | Furin-like\|PF00757.11 | | 101592 | Gastric Cancer |
| 2065 | 284G > R | Furin-like\|PF00757.11 | | 96115 | Colorectal Cancer |
| 2065 | 284G > R | Furin-like\|PF00757.11 | | 94592 | Colorectal Cancer |
| 2065 | 284G > R | Furin-like\|PF00757.11 | | 96562 | Colorectal Cancer |
| 2065 | 295V > A | Furin-like\|PF00757.11 | | 96737 | Colorectal Cancer |
| 2065 | 325G > R | Furin-like\|PF00757.11 | | 96115 | Colorectal Cancer |
| 2065 | 325G > R | Furin-like\|PF00757.11 | | 96115 | Colorectal Cancer |
| 2065 | 432I > I | Recep_L_domain\|PF01030.15 | | 98204 | Gastric Cancer |
| 2065 | 492D > H | Toxin_7\|PF05980.3 | | 100695 | Non-Small Cell Lung Cancer |
| 2065 | 536L > L | | | 90574 | Ovarian Cancer |
| 2065 | 714V > M | Pkinase\|PF00069.16, Pkinase_Tyr\|PF07714.8 | | 86582 | Non-Small Cell Lung Cancer |
| 2065 | 809Q > R | Pkinase_Tyr\|PF07714.8, Pkinase\|PF00069.16 | | 101592 | Gastric Cancer |
| 2065 | 846S > I | Pkinase\|PF00069.16, Pkinase_Tyr\|PF07714.8 | | 101763 | Colorectal Cancer |
| 2065 | 1164T > A | | | 95504 | Colorectal Cancer |
| 2065 | 1301Q > Q | | | 96630 | Colorectal Cancer |
| 2065 | 104V > M | | | 94120 | Gastric Cancer |
| 2065 | 104V > M | | | 98988 | Gastric Cancer |
| 2065 | 111Y > C | | | 94271 | Gastric Cancer |
| 2065 | 135R > L | | | 94138 | Gastric Cancer |
| 2065 | 232A > V | | | 94128 | Gastric Cancer |
| 2065 | 406M > T | | | 94117 | Gastric Cancer |
| 2065 | 453R > H | | | 94255 | Gastric Cancer |
| 2065 | 498K > I | | | 94137 | Gastric Cancer |
| 2065 | 1089R > W | | | 92177 | Gastric Cancer |

*Genomic positions based on version NCBI R37
WES = whole exome sequencing

TABLE 3
Published ERBB3 mutations in human cancers

| | | # of | # of | % | Mutations (amino | |
|---|---|---|---|---|---|---|
| | Tissue Diagnosis | mutants | samples | Frequency | acid change) | Reference |
| 1 | Breast Cancer (HR+) | 3 | 65 | 4.62 | Q281H, T389R, E928G | Nature (2010) 466: 869 |
| 2 | NSCLC (Adeno) | 3 | 188 | 1.60 | G69R, G284R, Q298* | Nature (2008) 455: 1069 |
| 3 | Glioblastoma | 1 | 91 | 1.10 | S1046N | Nature (2008) 455: 1061 |
| 4 | Ovarian | 3 | 339 | 0.88 | V104M, V438I, D1149E | Nature (2007) 446: 153 [23 sampl [symbol] (23 + 316) |
| 5 | colon | 1 | 100 | 1.00 | S846I | Int J of Ca (2006) 119: 2986 |
| 6 | Head and Neck Cancer | 1 | 74 | 1.35 | M90I | Science (2011) - Epub data 2011/07/30 |

[symbol]

indicates data missing or illegible when filed

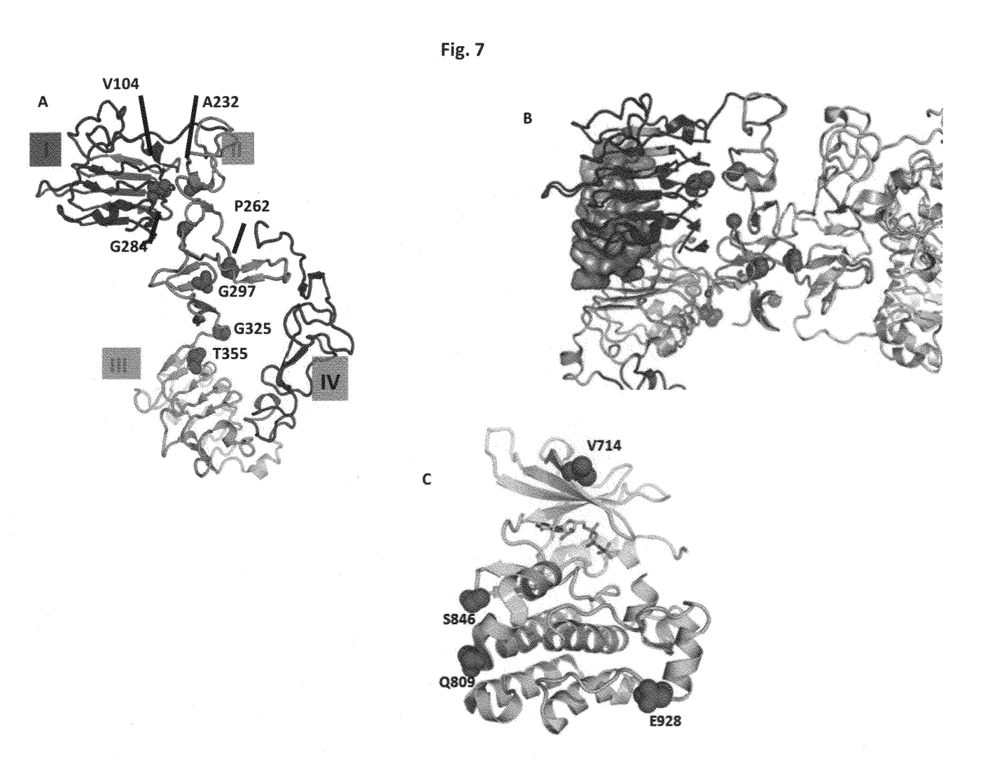

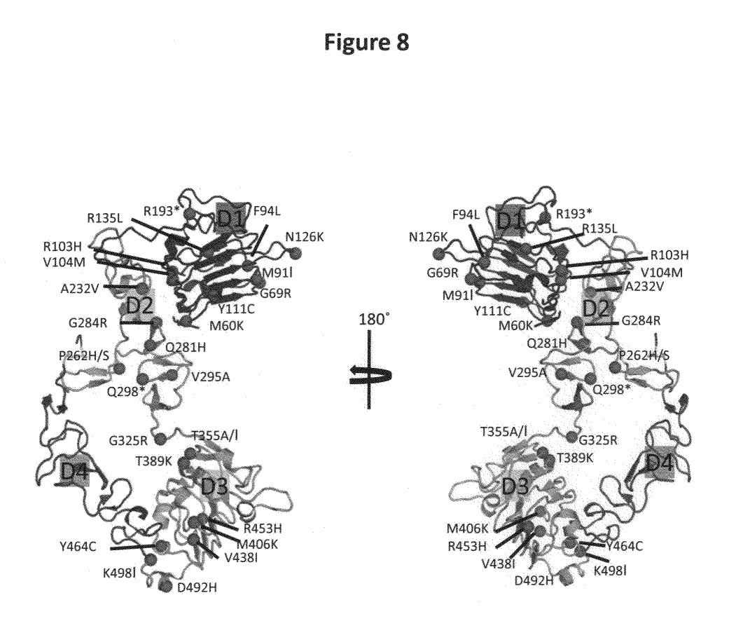

To further understand the mutations we mapped them to published ERBB3 ECD⁷ and kinase domain (Jura et al. Proceedings of the National Academy of Sciences 106, 21608-21613 (2009); Shi et al. Proceedings of the National Academy of Sciences 107, 7692-7697 (2010)) crystal structures (FIG. 7 and FIG. 8). Interestingly, the hotspot mutations at V104, A232 and G284 cluster in the domains I/II interface. The clustering of these three sites at the interface between domains II and III suggests they may act by a common mechanism. Domain II comprises several cystine-rich modules arranged like vertebrae. Small changes in the relationship amongst these semi-independent features have been assigned functional importance among family members (Alvarado et al. Nature 461, 287-291 (2009). The V104/A232/G284 mutations may shift one or more of these modules and cause an altered phenotype. The mutation at P262 is at the base of domain II, close to Q271 involved in the domain II/IV interaction required for the tethered, closed confirmation. Kinase domain mutations at residues 809 and 846 are homologous to positions proximal to the path taken by the C-terminal tail in the EGFR kinase structure, a segment that has been assigned a role in endocytosis. Sites of other mutations appear in FIG. 8.

ERBB3 Mutants Promote Ligand-Independent Proliferation of MCF10a Mammary Epithelial Cells

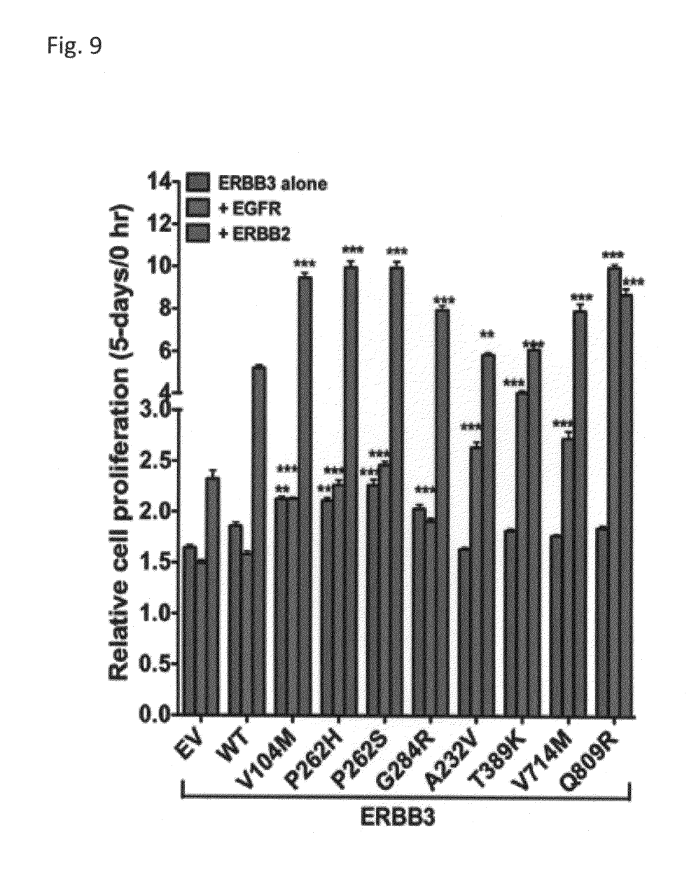

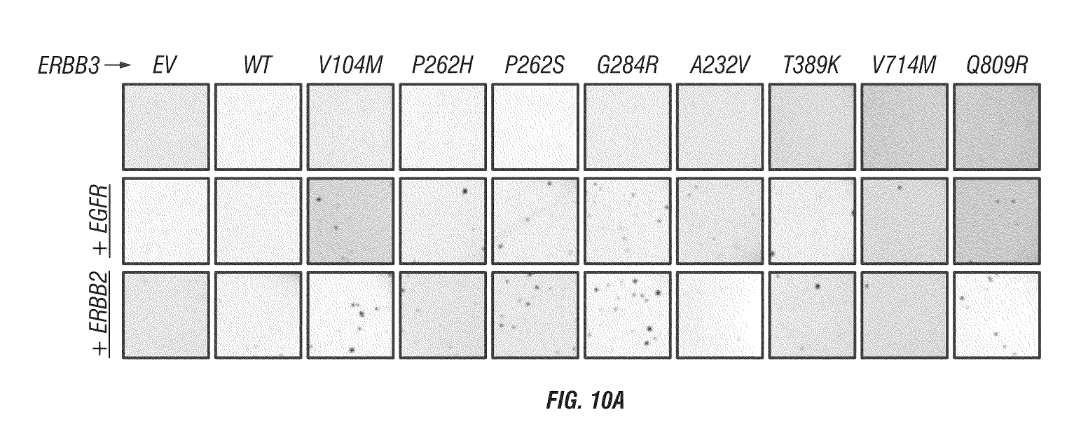

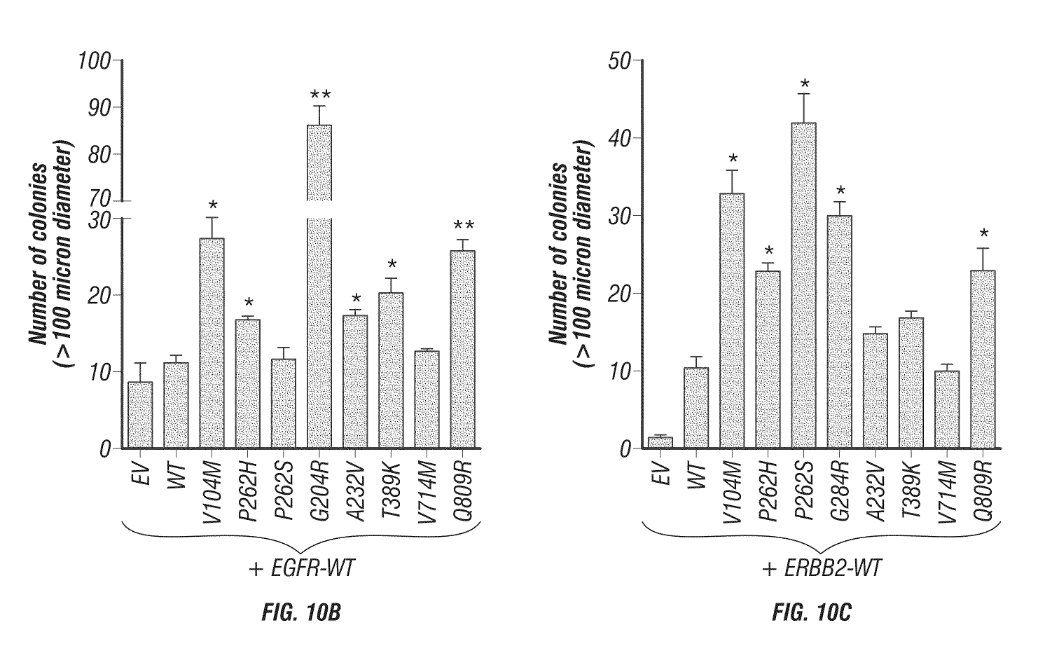

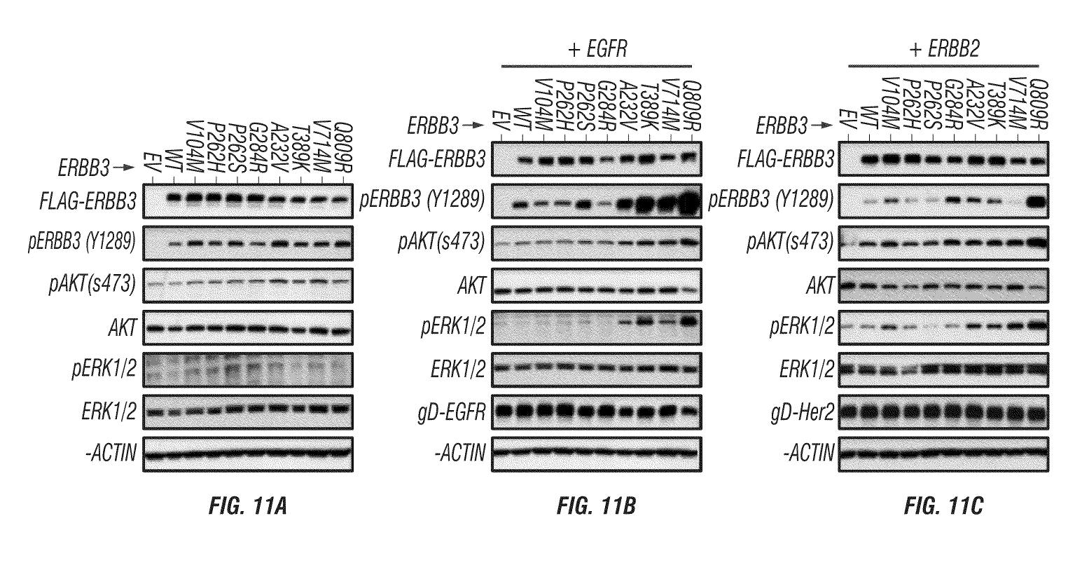

MCF-10A mammary epithelial cells require EGF for proliferation (Soule, H. D. et al. Cancer Res 50, 6075-6086 (1990); Petersen et al. Proceedings of the National Academy of Sciences of the United States of America 89, 9064-9068 (1992)). Oncogenes when expressed in MCF10A cells, can render them EGF-independent (Debnath et al. The Journal of cell biology 163, 315-326 (2003); Muthuswamy et al. Nat Cell Biol 3, 785-792 (2001)). In order to understand the oncogenic potential of the ERBB3 mutations we tested the ability of a select set of the ERBB3 mutants to support cellular transformation and proliferation. We tested six (V104M, A232V, P262H, P262S, G284R and T389K) ERBB3 ECD mutants including the four ECD-hotspot mutants and two (V714M and Q809R) ERBB3 kinase-domain mutants for their effects on cell proliferation, signaling, acinar formation, anchorage-independent growth and migration by stably expressing them in MCF10A cells. Since ERBB family members function as heterodimers in signaling and cellular transformation, we also tested the functional effects of ERBB3 mutants by co-expressing them with wild-type (WT) EGFR or ERBB2. We found that the ERBB3 mutants when expressed alone in MCF10A, in the absence of exogenous ERBB3 ligand NRG1 or EGF, showed very little increase in ligand-independent proliferation (FIG. 9), colony formation (FIG. 10) or elevation in signaling-activation status markers like pERBB3, pAKT and pERK (FIG. 11A) compared to ERBB3-WT. However, expression of ERBB3 mutants in combination with EGFR or ERBB2 showed a significant increase in proliferation and colony formation compared to ERBB3-WT (FIG. 9 and FIG. 10). In addition, majority of the ERBB3 mutants in combination with EGFR or ERBB2 led to elevated pERBB3, pAKT and pERK (FIGS. 11B and C).

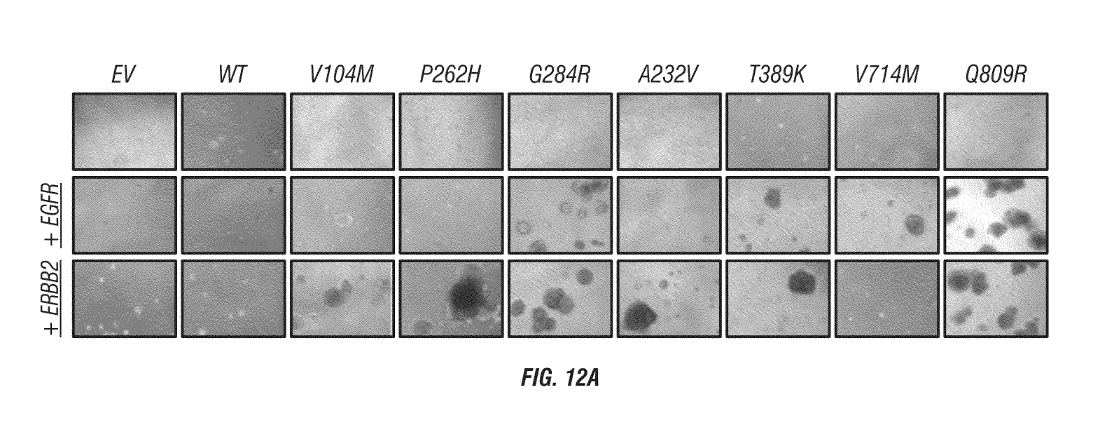

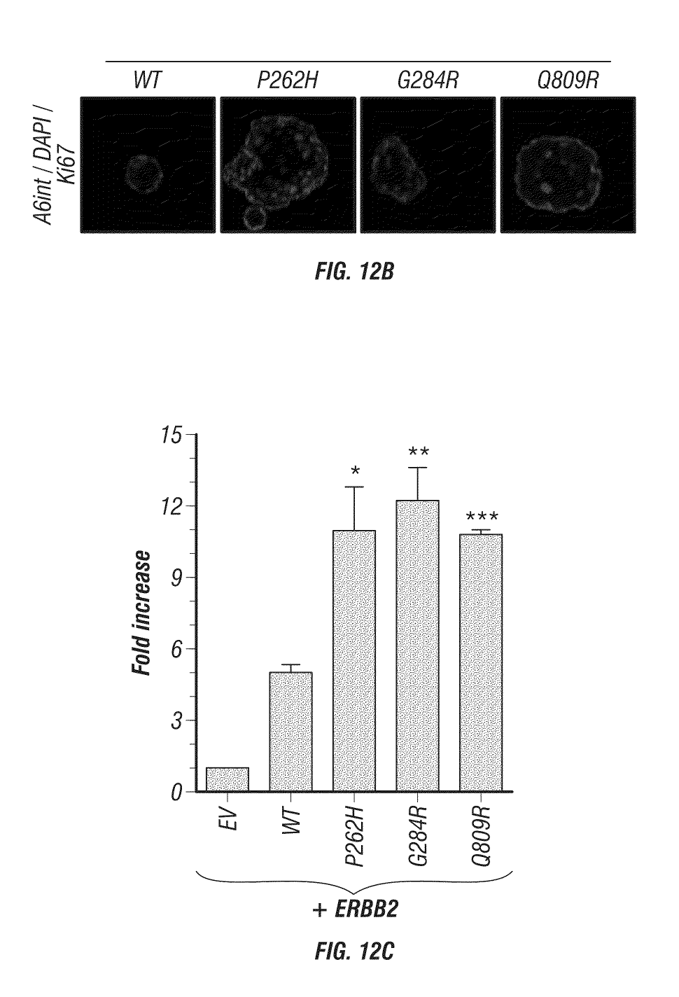

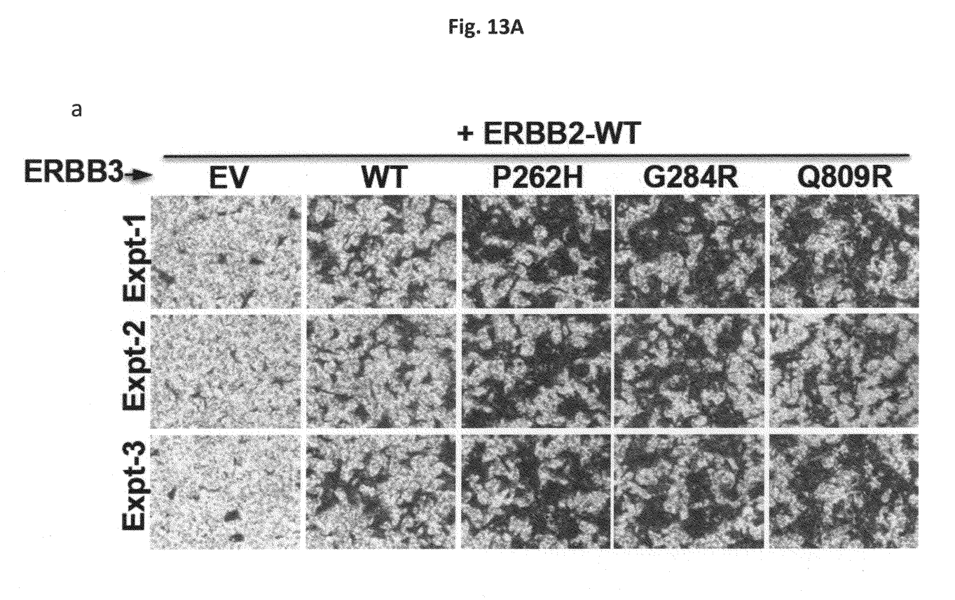

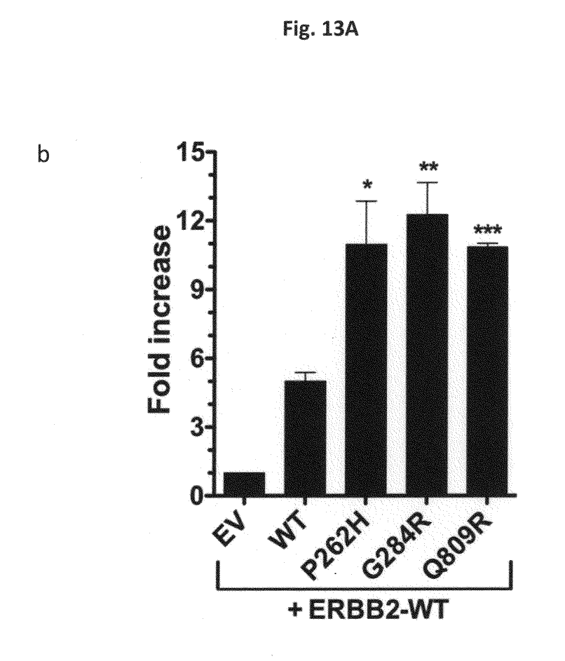

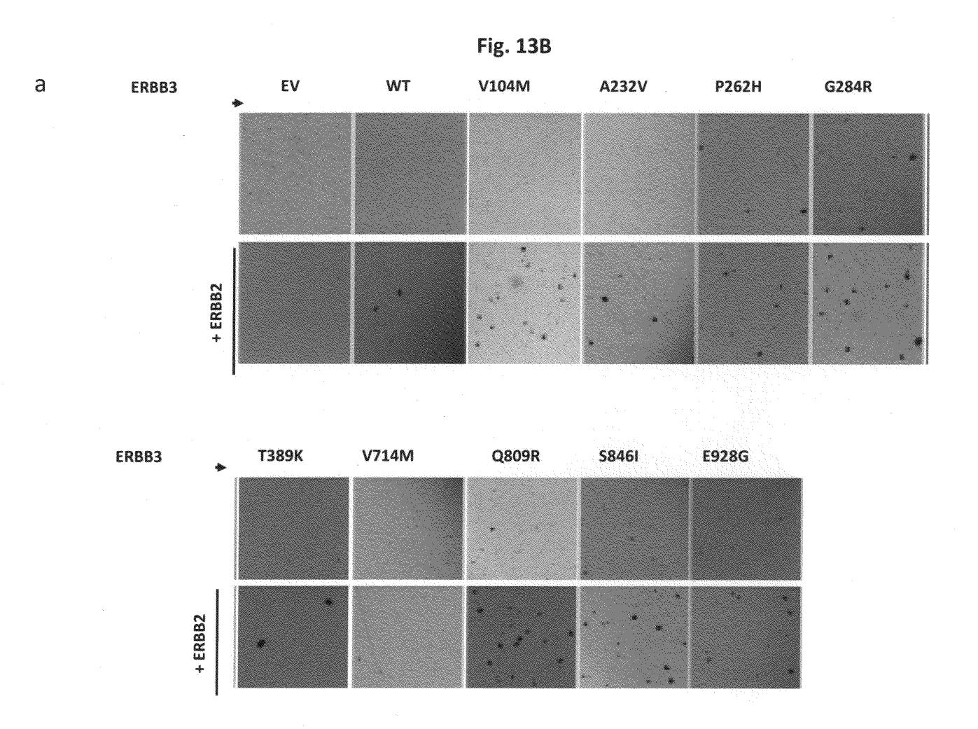

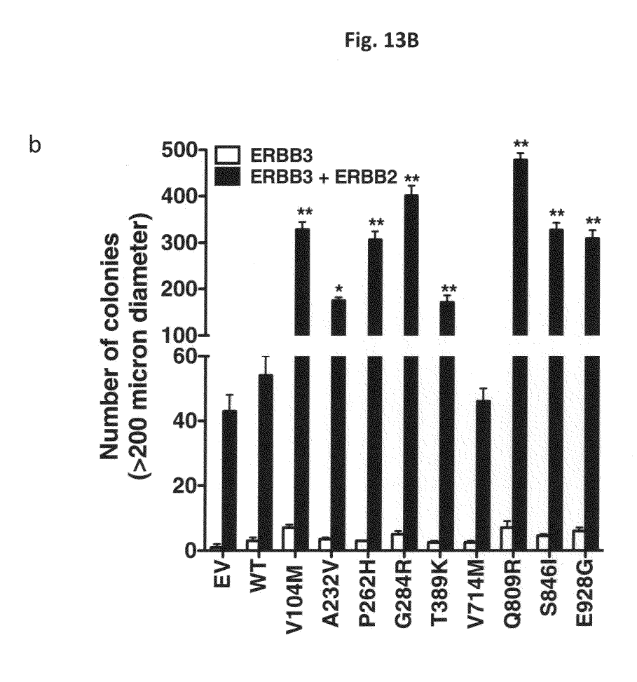

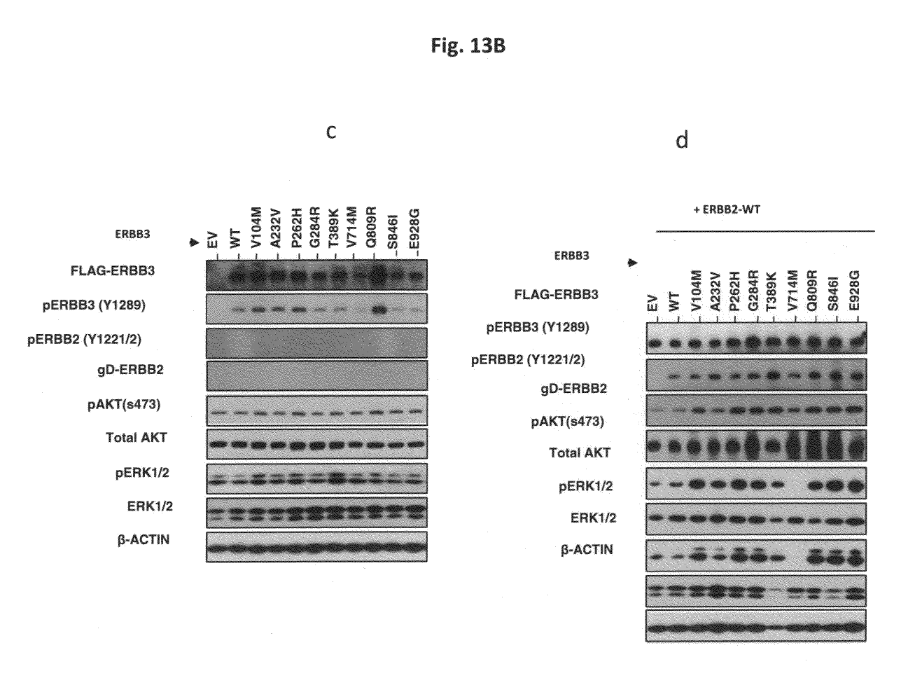

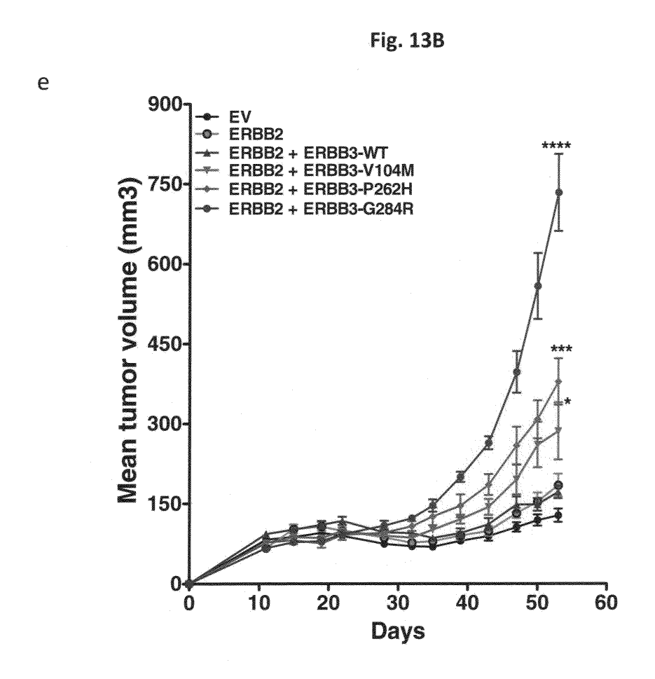

MCF10A cells form acinar-cell spheroids when cultured on reconstituted three dimensional (3D) basement membrane gel cultures, in the presence of EGF (Muthuswamy et al. Nat Cell Biol 3, 785-792 (2001); Muthuswamy Breast Cancer Research 13, 103 (2011)). However, expression of some oncogenes can render them EGF-independent and also result in complex multiacinar structures (Debnath et al. The Journal of cell biology 163, 315-326 (2003); Brummer et al. Journal of Biological Chemistry 281, 626-637 (2005); Bundy et al. Molecular Cancer 4, 43 (2005)). In 3D culture studies lacking serum, EGF and NRG1, ectopic expression of ERBB3 mutants in combination with EGFR or ERBB2 in MCF10A cells promoted large acinar structures, compared to MCF10A cells that co-express ERBB3-WT with EGFR or ERBB2 (FIG. 12A). Staining for Ki67, a marker for proliferation, in acini derived from ERBB3 mutant/ERBB2 co-expressing MCF10 cells showed increased proliferation in all the mutants tested (FIG. 12B). Further, the same MCF10A cells expressing a subset of the ERBB3-mutant/ERBB2 also showed increased migration (FIG. 12C and FIG. 13A) compared to ERBB3-WT/ERBB2 cells. These results taken together confirm the oncogenic nature of the ERBB3 mutants.

ERBB3 Mutants Promote Anchorage-Independent Growth of Colonic Epithelial Cells

IMCE are immortalized mouse colonic epithelial cells that can be transformed by expression of oncogenic Ras (D'Abaco et al. (1996). Mol Cell Biol 16, 884-891; Whitehead et al. (1993). PNAS 90, 587-591). We used IMCE cells and tested ERBB3 mutants for anchorage-independent growth, signaling and in vivo tumorigenesis by stably expressing the ERBB3 mutants either alone or in combination with ERBB2. As shown in FIG. 13B (a-b), we found that the ERBB3-WT or the mutants on their own, when expressed did not promote anchorage independent growth. However, a majority of the ERBB3 mutants, unlike the ERBB3-WT, when co-expressed with ERBB2 promoted anchorage independent growth (FIG. 13B (a-b)). Consistent with the anchorage independent growth observed, a majority of the IMCE cells expressing ERBB3 mutants along with ERBB2 showed elevated pERBB3 and/or pERBB2 and a concomitant increase in pAKT and/or pERK (FIG. 13B (c-d)). Although some of the ERBB3 mutants on their own showed elevated ERBB3 mutants, it did not promoted anchorage independent growth or downstream signaling. To further confirm that oncogenic activity of the ERBB3 mutants, we tested several hotspot ECD-mutant expressing cells for their ability to promote tumor growth in vivo. Consistent with their ability to support anchorage independent growth and signaling, IMCE cells co-expressing ERBB3 V104M, P262H or G284R, unlike WT, along with ERBB2 promoted tumor growth (FIG. 13B (e)).

ERBB3 Mutants Promote IL3-Independent Cell Survival and Transformation

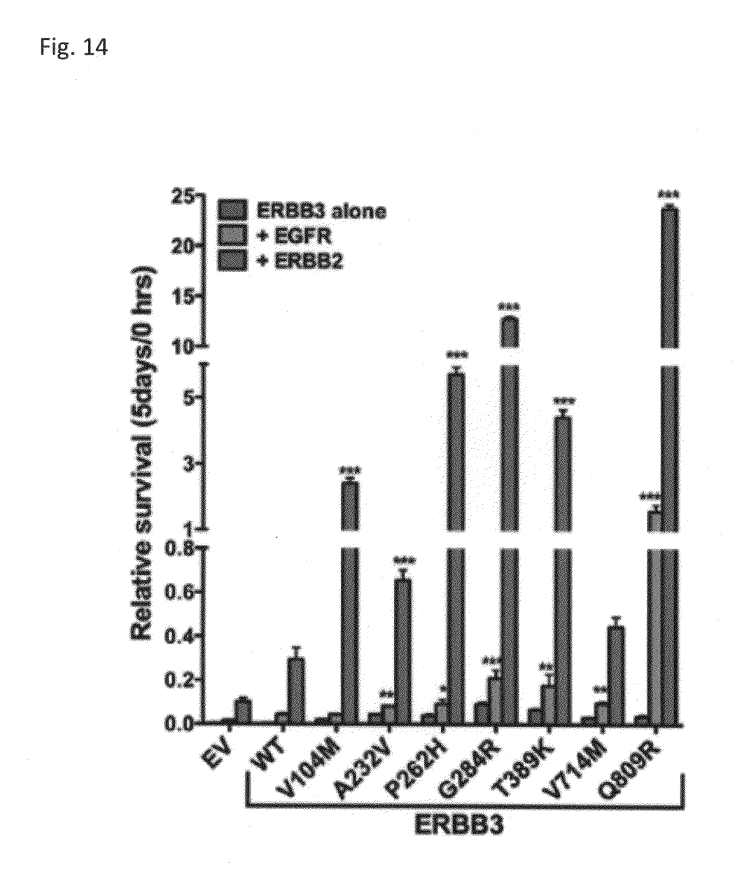

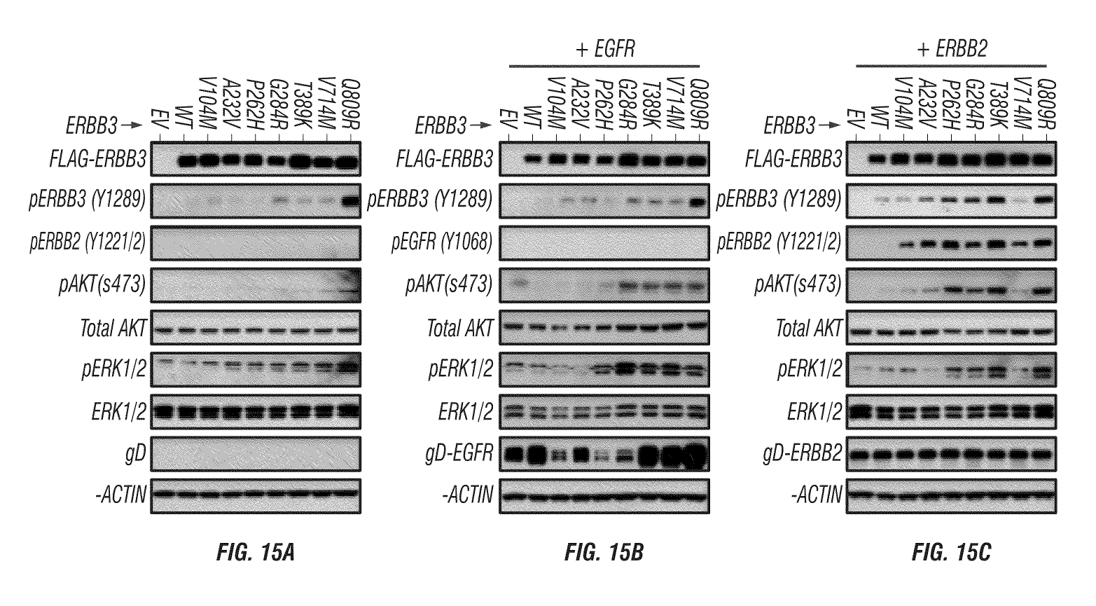

In order to further confirm the oncogenic relevance of the ERBB3 mutations we tested the ERBB3 mutants for their effects on signaling, cell survival and anchorage-independent growth by stably expressing them either alone or in combination with EGFR or ERBB2 in IL-3 dependent BaF3 cells. BaF3 is an interleukin (IL)-3 dependent pro-B cell line that has been widely used to study oncogenic activity of genes and development of drugs that target oncogenic drivers (Lee et al. (2006). PLoS medicine 3, e485; Warmuth et al. (2007) Current opinion in oncology 19, 55-60). While the ERBB3 mutants promoted little or no IL-3-independent survival of BaF3 cells when expressed alone, they were far more effective than WT-ERBB3, when co-expressed in combination with EGFR-WT or ERBB2-WT (FIG. 14 and FIG. 15A,B). ERBB3 mutants, co-expressed with ERBB2, were ˜10-50 fold more effective in promoting IL-3 independence survival than when co-expressed with EGFR (FIG. 14). This is consistent with previous studies that show ERBB3-ERBB2 heterodimers, formed following activation, to be among the most potent activators of cell signaling (Pinkas-Kramarski et al. The EMBO journal 15, 2452-2467 (1996); Tzahar et al. Molecular and cellular biology 16, 5276-5287 (1996); Holbro et al. PNAS 100, 8933-8938 (2003)). Interestingly, the Q809R kinase domain mutant, in combination with ERBB2 or EGFR was the more effective in promoting IL-3 independent survival of BaF3 cells, than any of the ECD mutants tested. Consistent with the IL-3-independent cell survival activity observed, a majority of the ERBB3 mutants showed increased phosphorylation, a signature of active ERBB receptors, when expressed alone or in combination with ERBB2 or EGFR (FIG. 15A-C). Further, the ERBB3 mutants co-expressed with ERBB2 showed elevated p-ERBB2 (Y1221/2), compared to the ERBB3-WT (FIG. 15C). Also, in combination with EGFR or ERBB2, a majority of the ERBB3 mutations showed elevated p-AKT and p-ERK levels, consistent with constitutive downstream signaling by the ERBB3 mutants (FIG. 15B,C). Having established the ability of the ERBB3 mutants to promote IL3-independent survival of BaF3 cells, we next investigated the ability of these mutants to promote anchorage-independent growth. We found that the BaF3 cells stably expressing P262H, G284R and Q809R ERBB3-mutants in combination with ERBB2 promoted robust anchorage-independent growth compared to ERBB3-WT (FIG. 16). Although several of the mutants promoted some anchorage-independent growth when expressed with EGFR, the effect was not as pronounced as observed in combination with ERBB2. This is consistent with previous reports that establish the requirement for ERBB3 in ERBB2-mediated oncogenic signaling (Holbro et al. PNAS 100, 8933-8938 (2003); Lee-Hoeflich et al. Cancer Research 68, 5878-5887 (2008)).

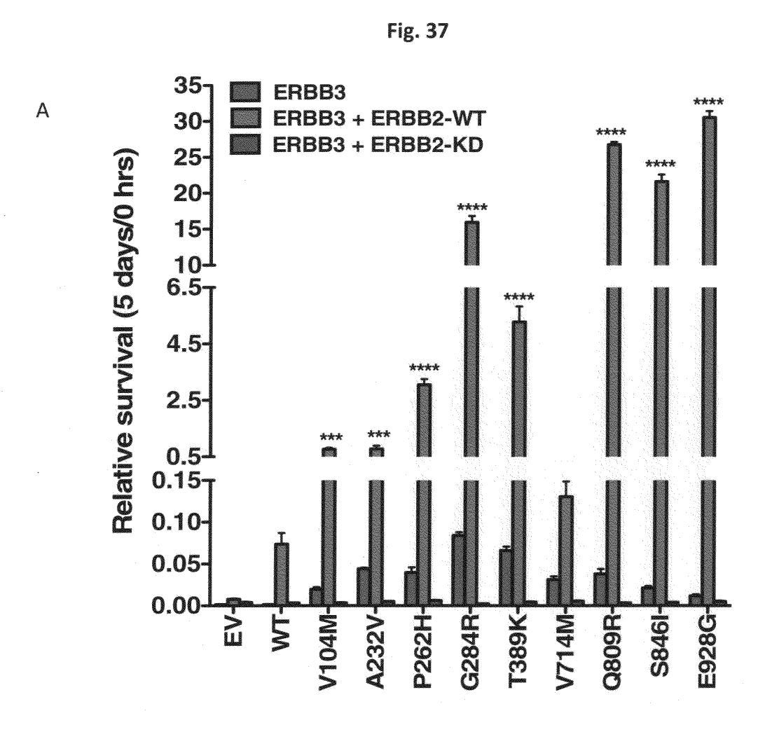

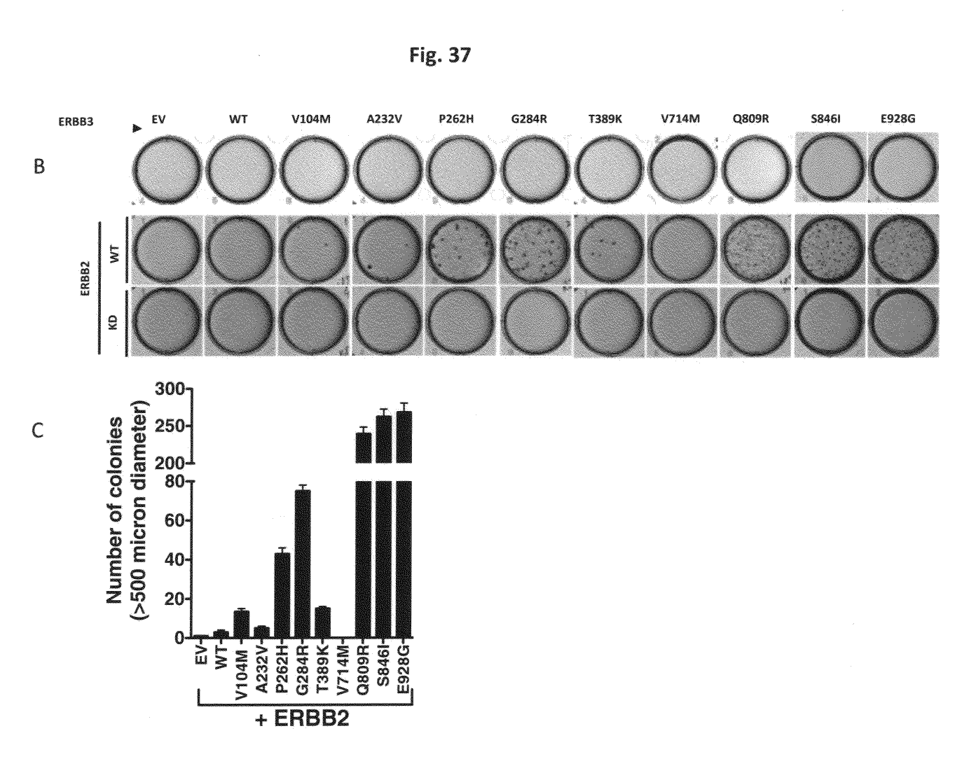

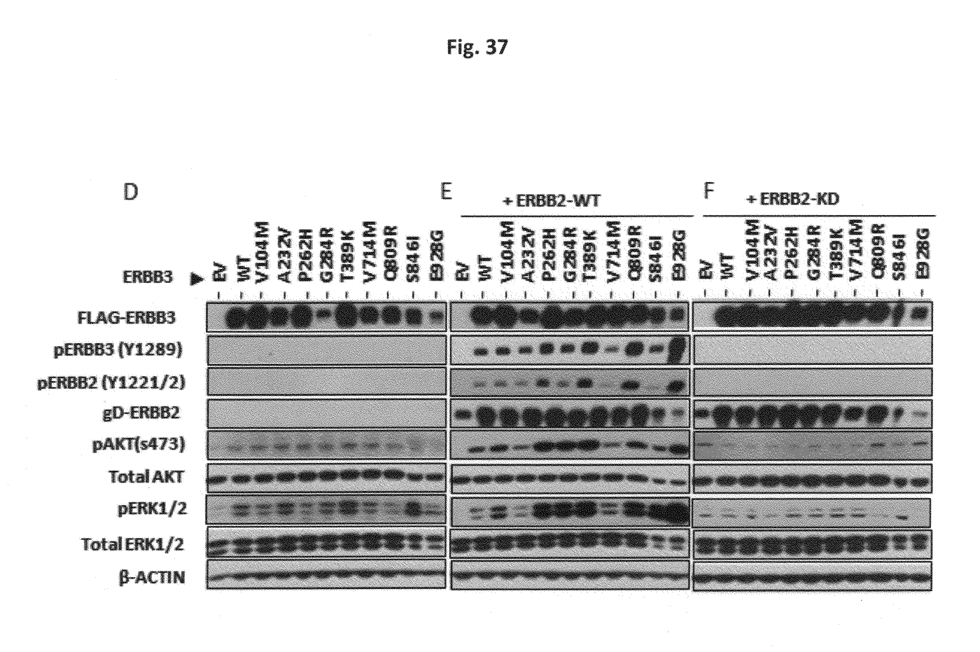

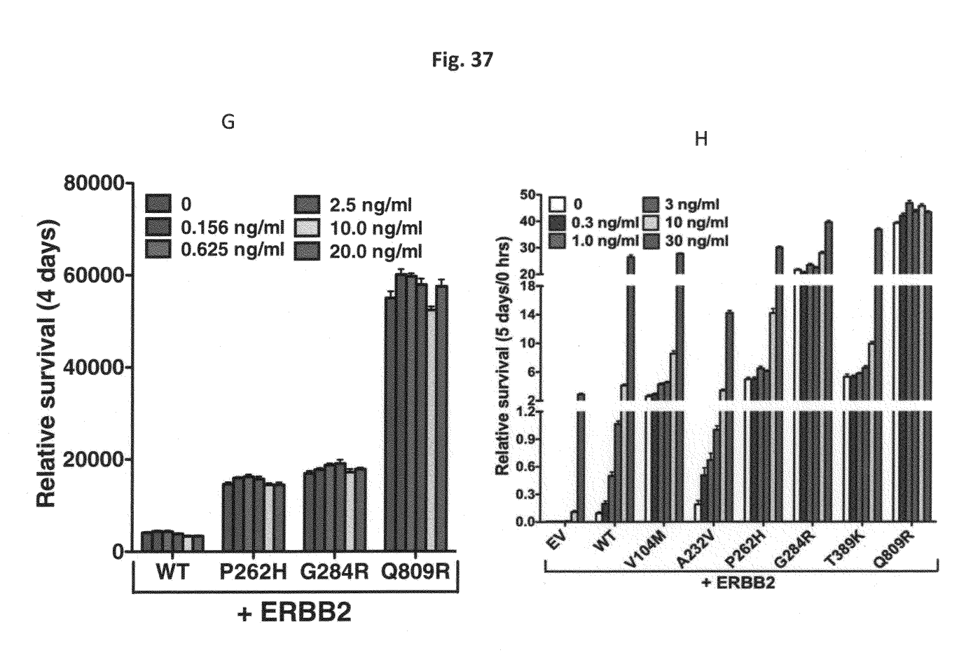

The BaF3 system was used to test several ERBB3 ECD mutants (V104M, A232V, P262H, P262S, G284R and, T389K) that included six ECD-hotspot mutants and four ERBB3 kinase-domain mutants (V714M, Q809R, S846I and E928G) for their effects on IL-3 independent cell survival, signaling, and anchorage-independent growth by stably expressing the ERBB3 mutants either alone or in combination with ERBB2. ERBB3 is kinase impaired and following ligand binding it preferentially forms heterodimers with ERBB2 to promote signaling (Holbro et al. (2003) supra; Karunagaran et al. (1996). The EMBO journal 15, 254-264; Lee-Hoeflich et al. (2008) supra; Sliwkowski et al. (1994) supra). Consistent with this, in the absence of exogenous ligand, ERBB3 wild type (WT) and the ERBB3 mutants on their own did not promote IL-3-independent survival of BaF3 cells (FIG. 37A). However, in the absence of exogenous ERBB3 ligand, the ERBB3 mutants, unlike ERBB3-WT, promoted IL3-independent BaF3 cell survival when co-expressed with ERBB2 (FIG. 37A), indicting the ERBB3 mutants may function in a ligand independent fashion. The cell survival activity of ERBB3 mutants was abrogated when they were co-expressed with a kinase dead (KD) ERBB2 K753M mutant, confirming the requirement for a kinase active ERBB2 (FIG. 37A). We further investigated ERBB3 mutants for their ability to promote anchorage-independent growth. The ERBB3 mutants, as observed in the survival assay, on their own did not support anchorage independent growth (FIG. 37B). However, we found that a majority of the ERBB3-mutants tested in combination with ERBB2, promoted anchorage-independent growth when compared to ERBB3-WT/ERBB2 expressing BaF3 cells (FIG. 37B-C). The anchorage-independent growth promoted by ERBB3 was confirmed dependent on that kinase activity of ERBB2, as the ERBB3 mutants in combination with ERBB2-KD did not promote colony formation (FIG. 37B-C). Western blot analysis of the BaF3 cells showed that the expression of ERBB3 mutants in combination with ERBB2 led to an increase in pERBB3, pERBB2, pAKT and/or pERK compared to ERBB3-WT (FIG. 37D-F). Consistent with the lack of cell survival activity or anchorage independent growth, the ERBB3 mutants on their own or in combination with ERBB2-KD did not show elevated pERBB2 and/or pAKT/pERK (FIG. 37D-F), though ERBB3 mutants on their own showed some elevated pERBB3 levels which likely due to endogenous ERBB2 expressed by BaF3 cells. In combination with ERBB2, the ERBB3 V714M kinase domain mutant consistent with its weak signaling showed only a modest cell survival activity and no anchorage independent growth (FIG. 37A-C). In contrast, the most active Q809R mutant in combination with ERBB2 showed robust downstream signaling compared to ERBB3-WT (FIG. 37A-C).

Ligand-Independent Oncogenic Signaling by ERBB3 Mutants

In an effort to understand the mechanism by which the ERBB3 mutants promote oncogenic signaling, we tested the ligand dependency of the ERBB3 mutants using our BaF3 system.

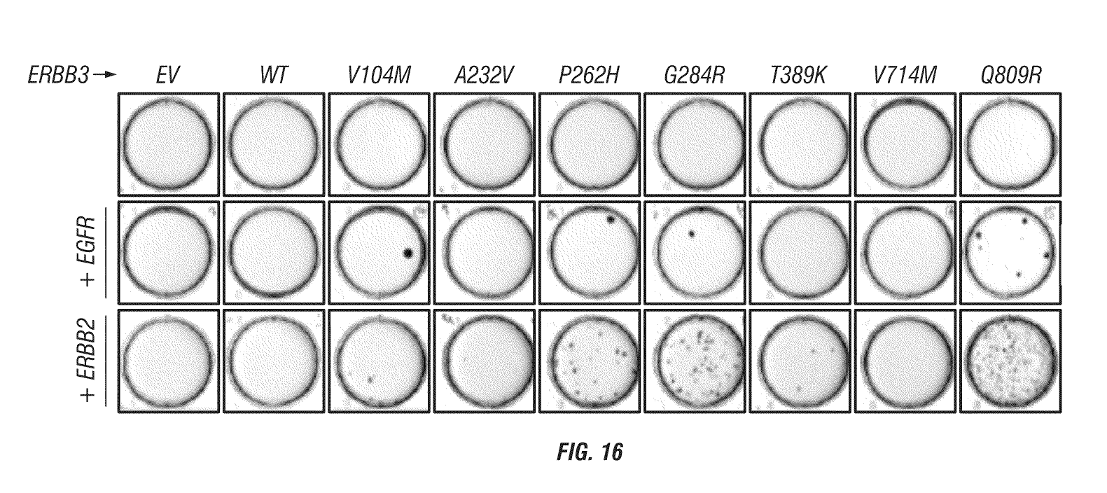

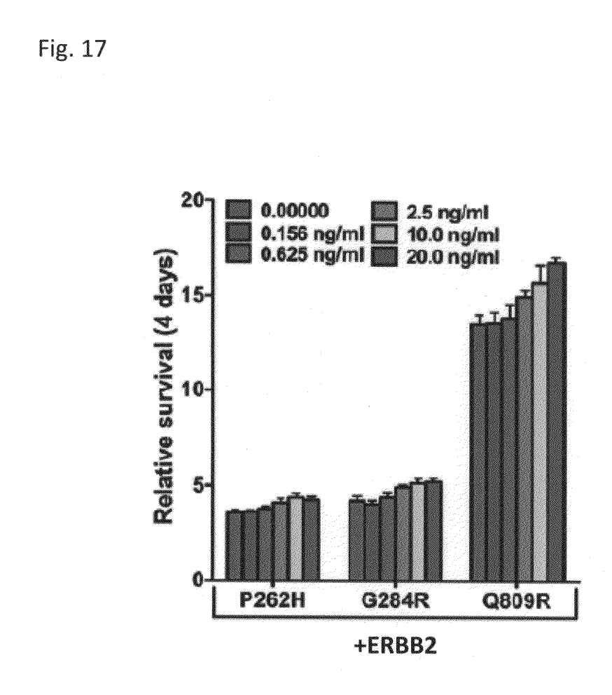

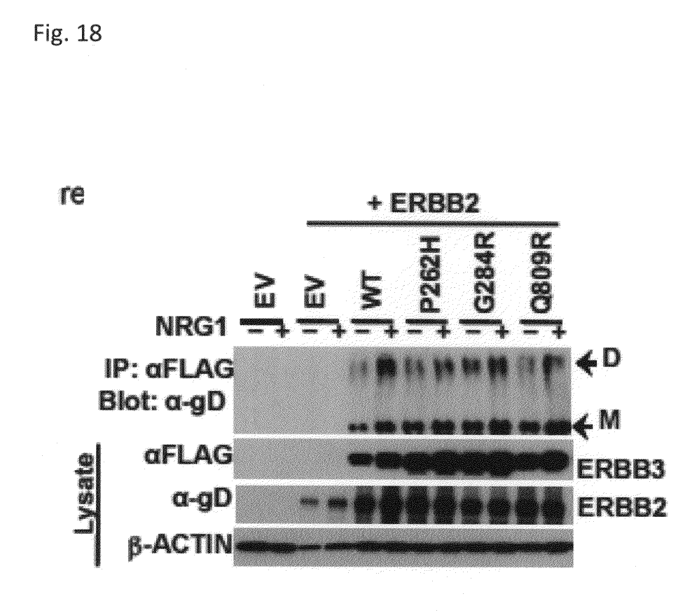

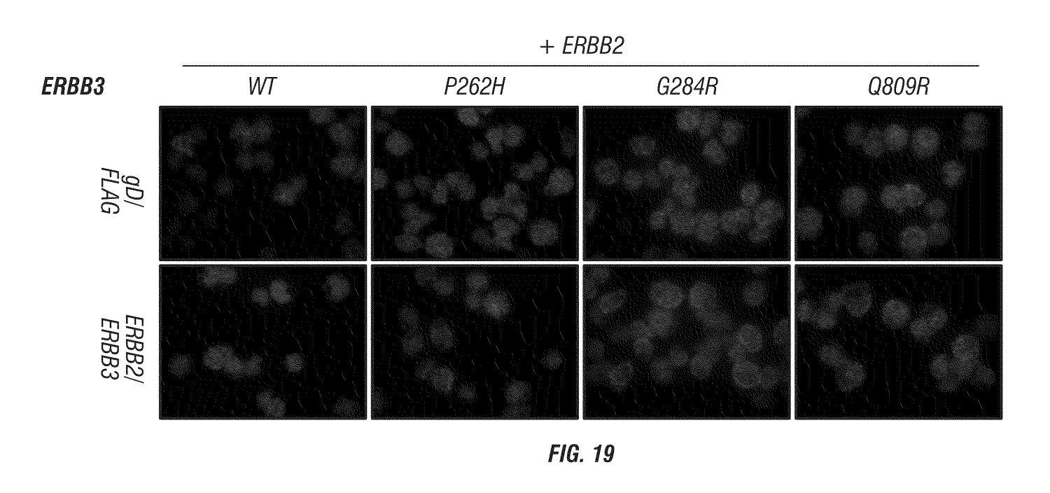

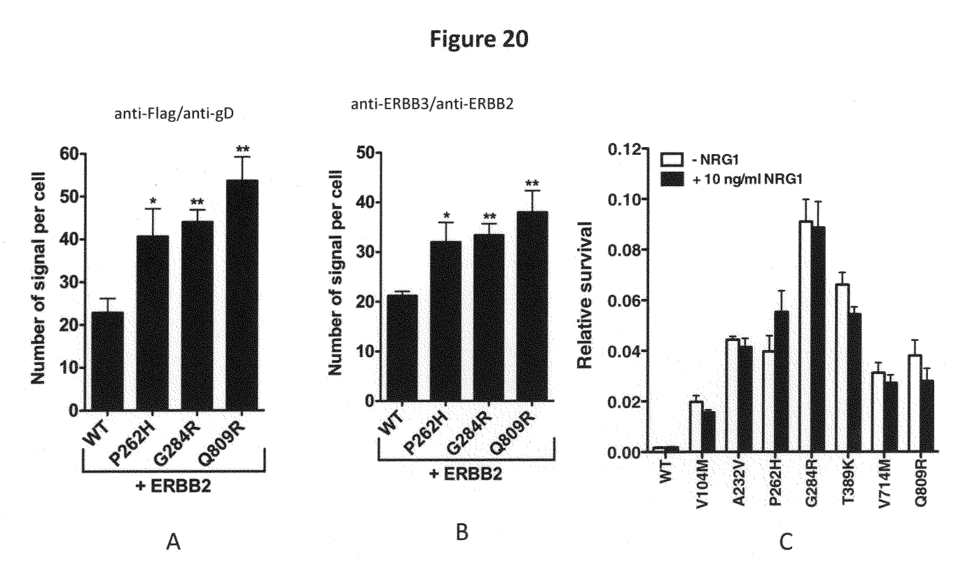

To establish the ligand-independent signaling by the ERBB3 mutants we tested their ability to promote IL-3-independent BaF3 survival under increasing dose of anti-NRG1 antibody, an ERBB ligand neutralizing antibody. We found that the addition of a NRG1 neutralizing antibody (Hegde et al. Manuscript submitted (2011) had no adverse effect on the ability of the ERBB3-mutants to promote IL-3 independent survival or anchorage independent colony formation (FIG. 17). Consistent with this, in immunopreciptation performed following cell surface receptor crosslinking, we found evidence for increased levels of ERBB3-mutant/ERBB2 heterodimers, in the absence of ligand, compared to the BaF3 cells co-expressing ERBB3-WT and ERBB2 (FIG. 18). This was further confirmed by the elevated levels of cell surface heterodimers in BaF3 cells expressing ERBB3-mutant/ERBB2, cultured in the absence of IL-3 or NRG1, using a proximity ligation assay (Soderberg et al. Nat Methods 3, 995-1000 (2006)) (FIG. 19 and FIG. 20A-B) when compared to cells expressing ERBB3-WT/ERBB2. These data suggest that the ERBB3 mutants, in combination with ERBB2, are capable of promoting IL-3 survival of BaF3 in a NRG1 independent manner.

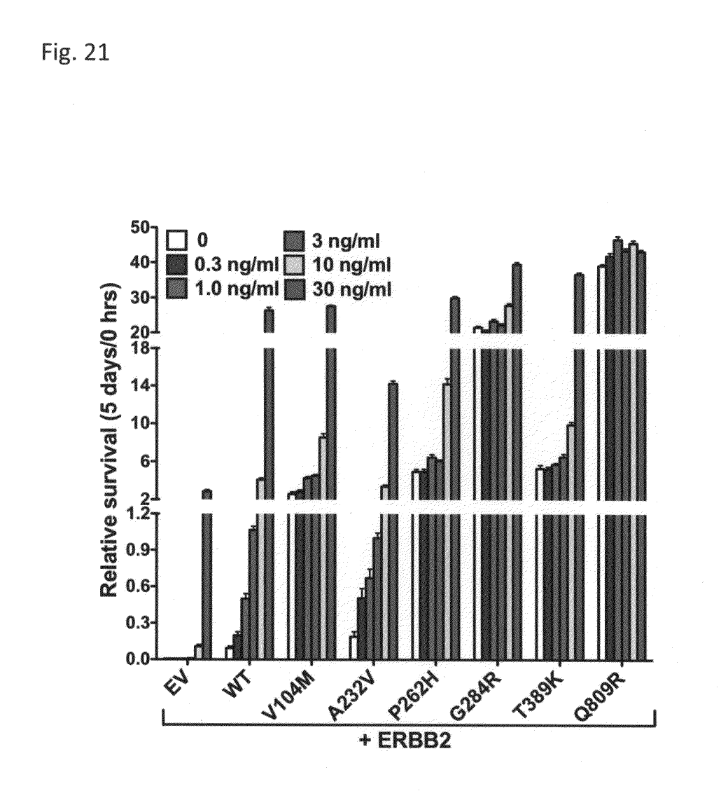

Having established that the ERBB3 mutants can signal independent of ligand, we tested if their activity could be augmented by ligand addition. We found that NRG1 was unable to support survival of BaF3 cells expressing ERBB3-WT or the mutants alone (FIG. 20C). However, at the highest concentration tested, increased the IL-3-independent survival of BaF3 cells expressing a majority of the ERBB3 mutants along with ERBB2, in a manner similar to the ERBB3-WT/ERBB2 expressing cells (FIG. 21). Interestingly, the A232V ERBB3 mutant, like the WT ERBB3, showed a NRG1 dose-dependent IL-3-independent survival response (FIG. 21). In contrast, G284R and Q809R did not show a significant increase in survival following ligand addition when compared to untreated cells expressing these mutants. The minimal response to ligand addition by G284R ECD and Q809R kinase domain mutants suggests a dominant role for the ligand-independent mode of signaling by these mutants (FIG. 21). Consistent with this, following ligand addition, while the P262H and the WT ERBB3 showed elevated heterodimer formation, the G284R ECD mutant and the Q809R kinase domain mutant showed only a modest increase in heterodimer formation when compared to the unstimulated cells (FIG. 18). These results show that while all the ERBB3 mutants are capable of ligand-independent signaling, some of them are still capable of responding to ligand stimulation.

To further understand the mechanism by which the ERBB3 mutants promote oncogenic signaling, we tested the ligand dependency of the ERBB3 mutants in our BaF3 system by treating these cells with increasing dose of an ERBB3-ligand neutralizing anti-NRG1 antibody (Hegde et al. (2011) supra). We found that the addition of a NRG1 neutralizing antibody (Id.) had no effect on the ability of the ERBB3-mutants to promote IL-3 independent survival (FIG. 37G). In FIG. 37H, ERBB3 ECD mutants show increased IL-3 independent BaF3 survival in response to increasing dose of exogenous NRG1.

ERBB3 Mutants Promote Oncogenesis In Vivo

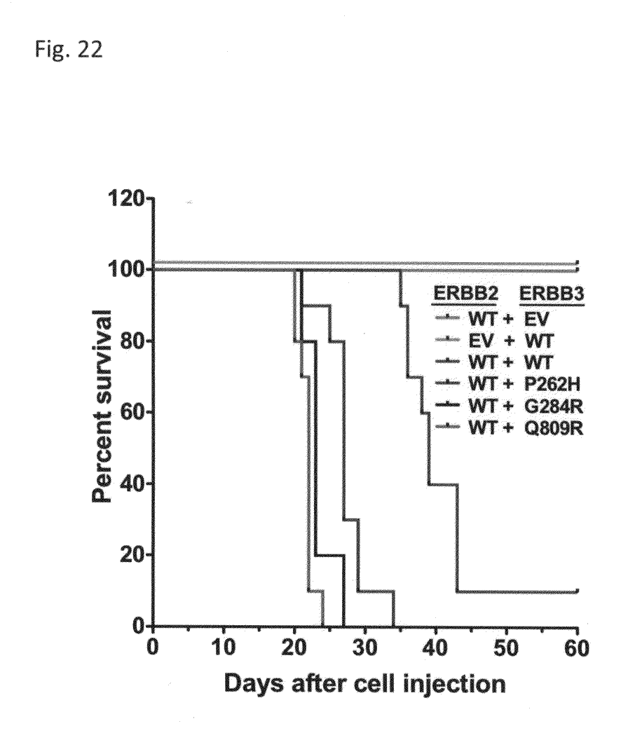

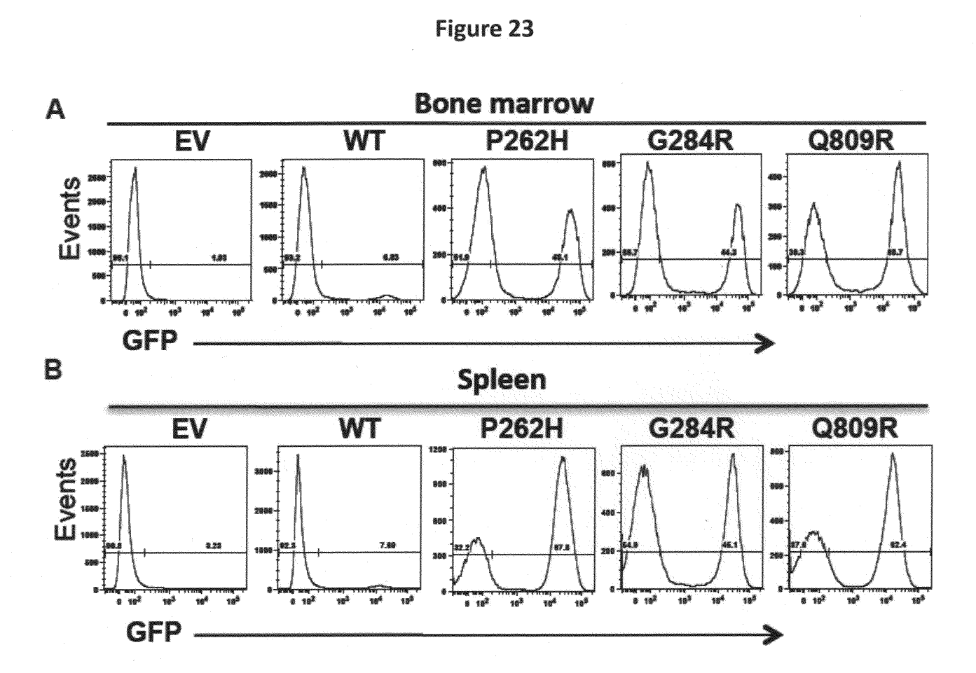

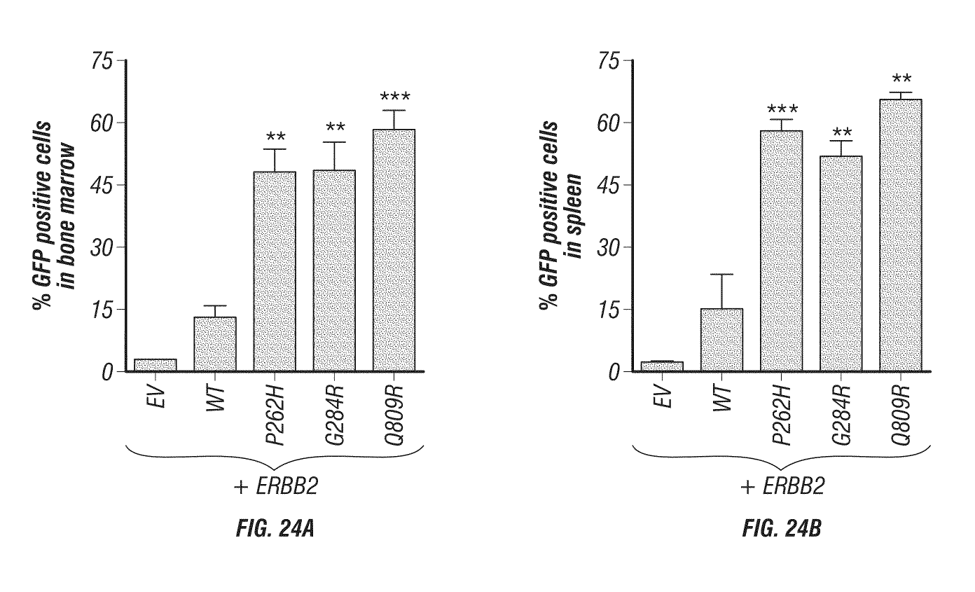

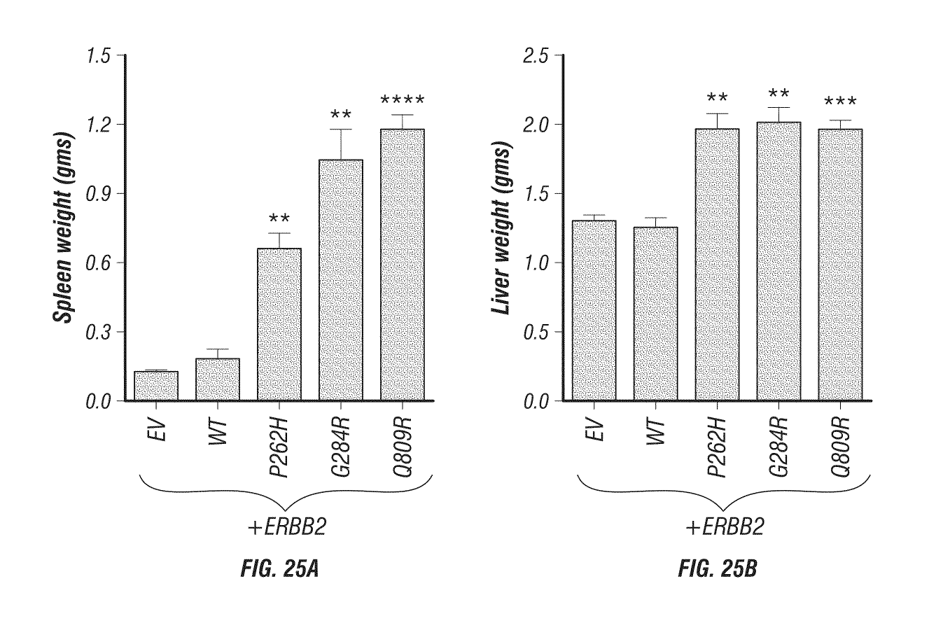

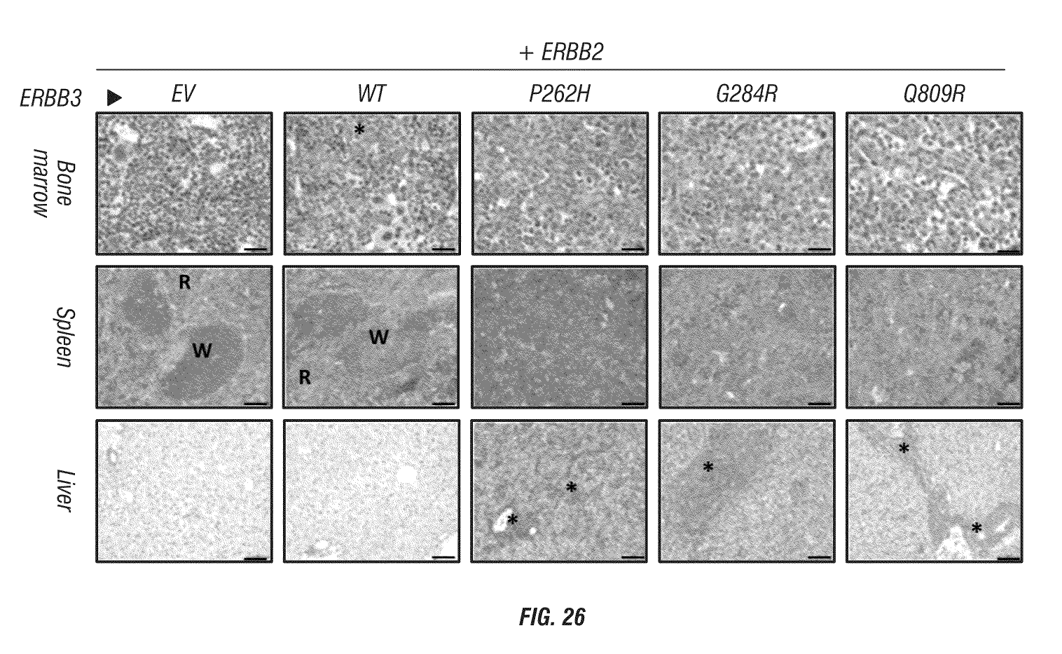

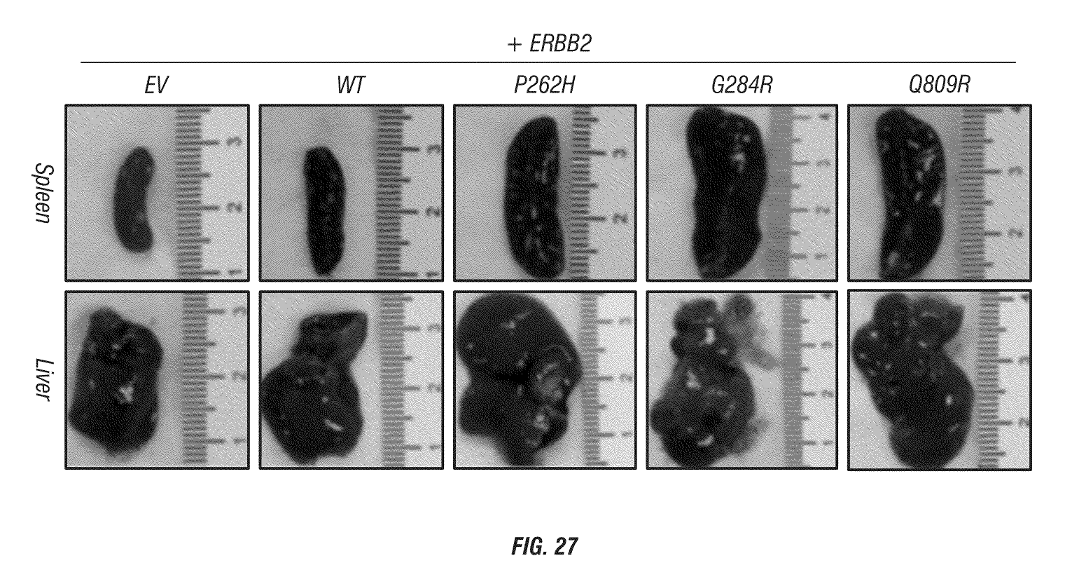

We and others have shown that BaF3 cells, rendered IL-3-independent by ectopic expression of oncogenes, promote leukemia-like disease when implanted in mice and lead to reduced overall survival (Horn et al. Oncogene 27, 4096-4106 (2008); Jaiswal et al. Cancer Cell 16, 463-474 (2009)). We tested the ability of BaF3 cells expressing ERBB3-WT, ECD-mutants (P262H or G284R) or the kinase domain ERBB3-mutant (Q809R) in combination with ERBB2 for their ability to promote leukemia-like disease. BaF3 cells transduced with ERBB3-WT alone or ERBB2 together with empty vector were used as controls. We found that mice transplanted with BaF3 cells expressing ERBB3 mutants together with ERBB2 showed a median survival of 22 to 27 days (FIG. 22). In contrast, mice receiving BaF3 cells expressing either ERBB3-WT alone or ERBB2 with empty vector were all alive at the end of the 60-day study period. However, animals receiving BaF3 cells co-expressing ERBB3-WT and ERBB2 developed leukemia like disease with a significantly longer latency (39 days; FIG. 22). Though the ERBB3-WT/ERBB2 BaF3 cells in vitro did not show IL-3 independence, their activity in the animal model is likely due to the presence of growth factors and cytokines in the in vivo environment that can activate ERBB3-WT/ERBB2 dimers and in part due to ligand-dependent signaling reported for ERBB3-ERBB2 heterodimers (Junttila et al. Cancer Cell 15, 429-440 (2009)). To follow disease progression we conducted necropsies at 20 days on an additional cohort of three mice per treatment. Bone marrow, spleen, and liver samples from these animals were reviewed for pathological abnormalities. As the BaF3 cells were tagged with eGFP, we examined isolated bone marrow and spleen for infiltrating cells by fluorescence-activated cell sorting (FACS). Consistent with the decreased survival, bone marrow and spleen from mice transplanted with cells expressing ERBB3□ mutants/ERBB2 showed a significant proportion of infiltrating eGFP-positive cells compared with bone marrow and spleen from mice receiving ERBB3-WT or ERBB2/empty-vector control cells (FIGS. 23-26). Further, concordant with the longer latency observed, a very low level of infiltrating eGFP positive cells was detected in the liver and spleen from animals receiving ERBB3-WT/ERBB2-WT cells. Also, animals from the ERBB3 mutant/ERBB2 arm showed increased spleen (FIG. 25A and FIG. 27) and liver (FIG. 25B and FIG. 27) size and weight compared to empty vector control or ERBB3-WT/ERBB2 at 20 days, further confirming the presence of infiltration cells. Additionally, histological evaluation of hematoxylin and eosin (H&E) stained bone marrow, spleen and liver sections showed significant infiltration of blasts in animals with cells expressing ERBB3-mutant/ERBB2 when compared to control at day 20 (FIG. 26). These results demonstrate the in vivo oncogenic potential of the ERBB3 mutants.

Targeted Therapeutics are Effective Against ERBB3 Mutants

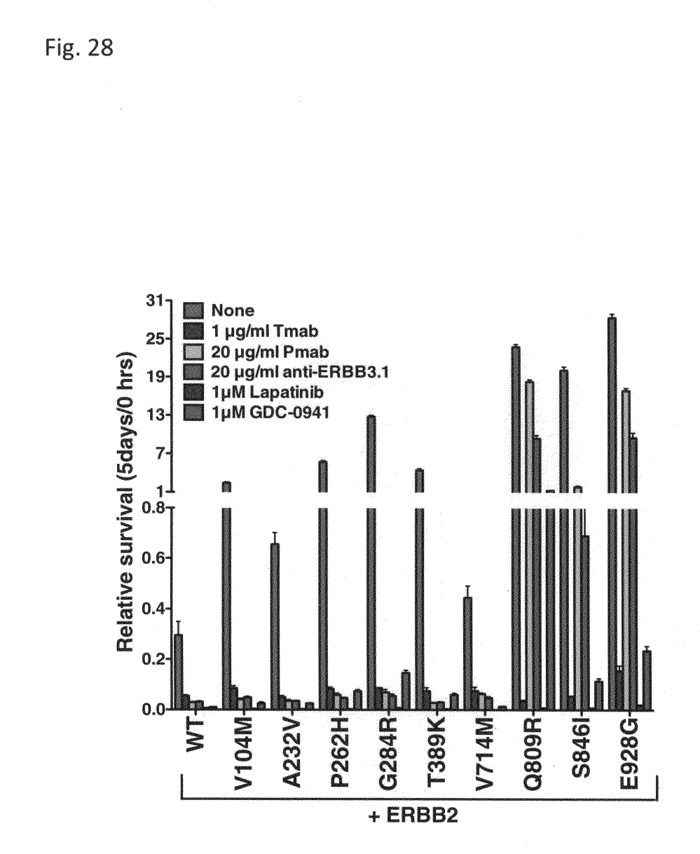

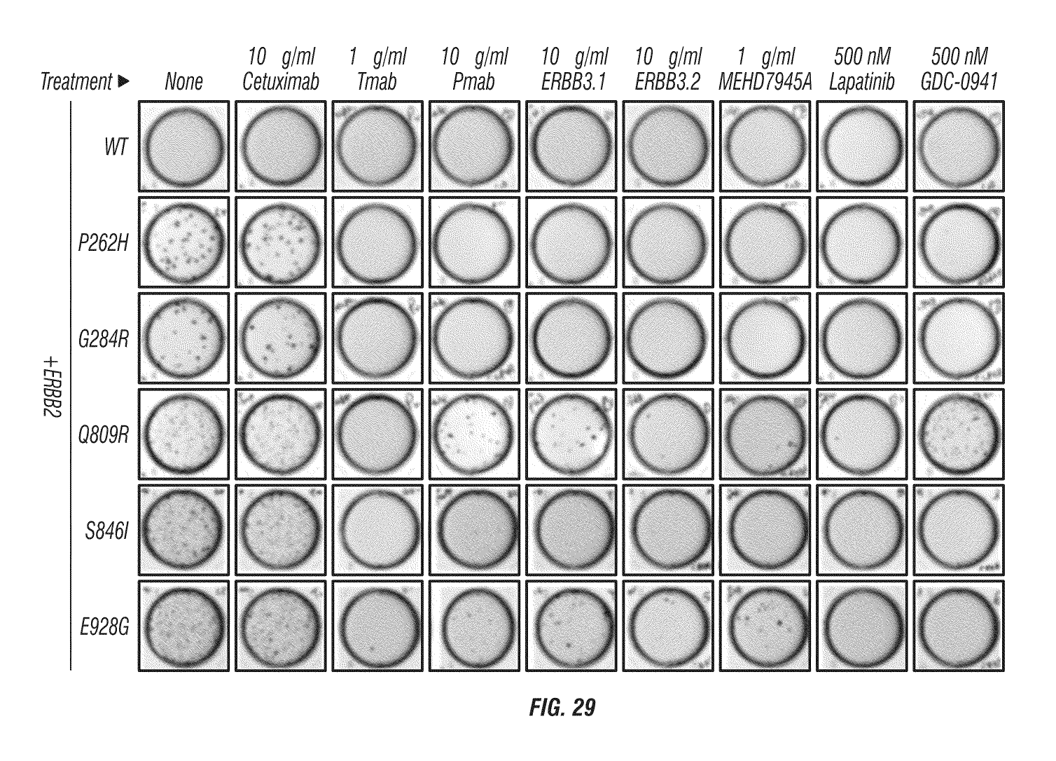

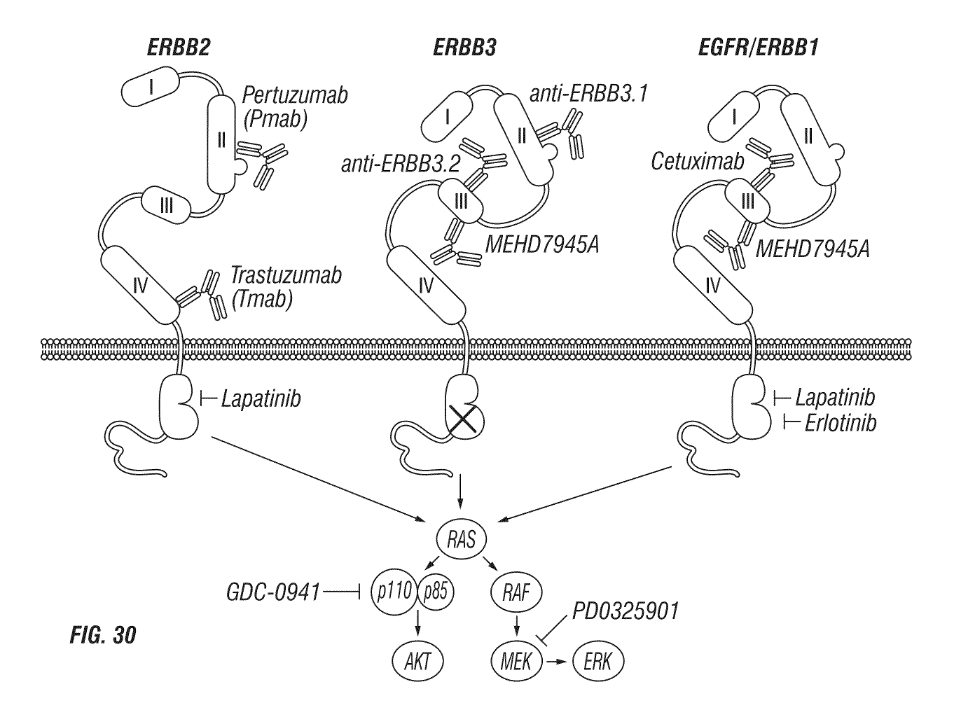

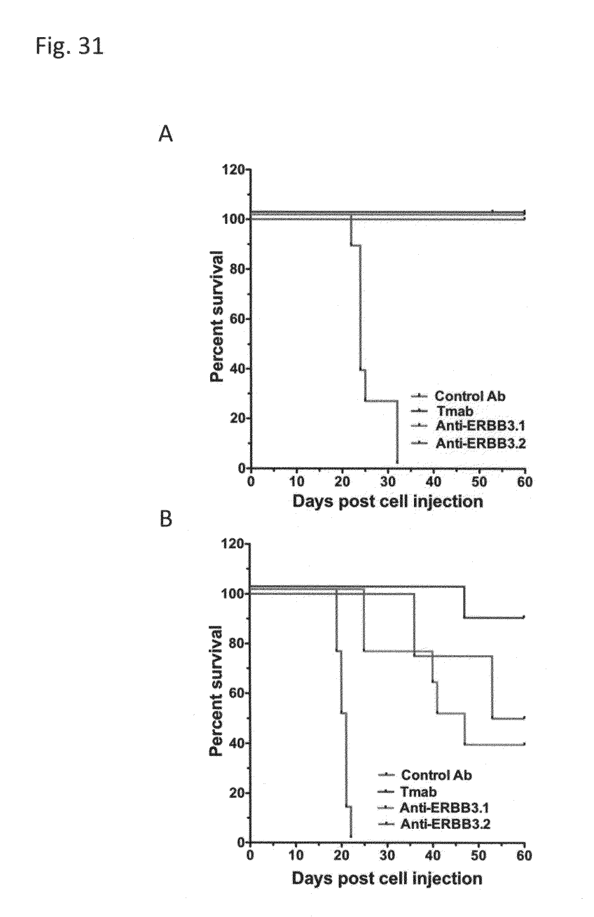

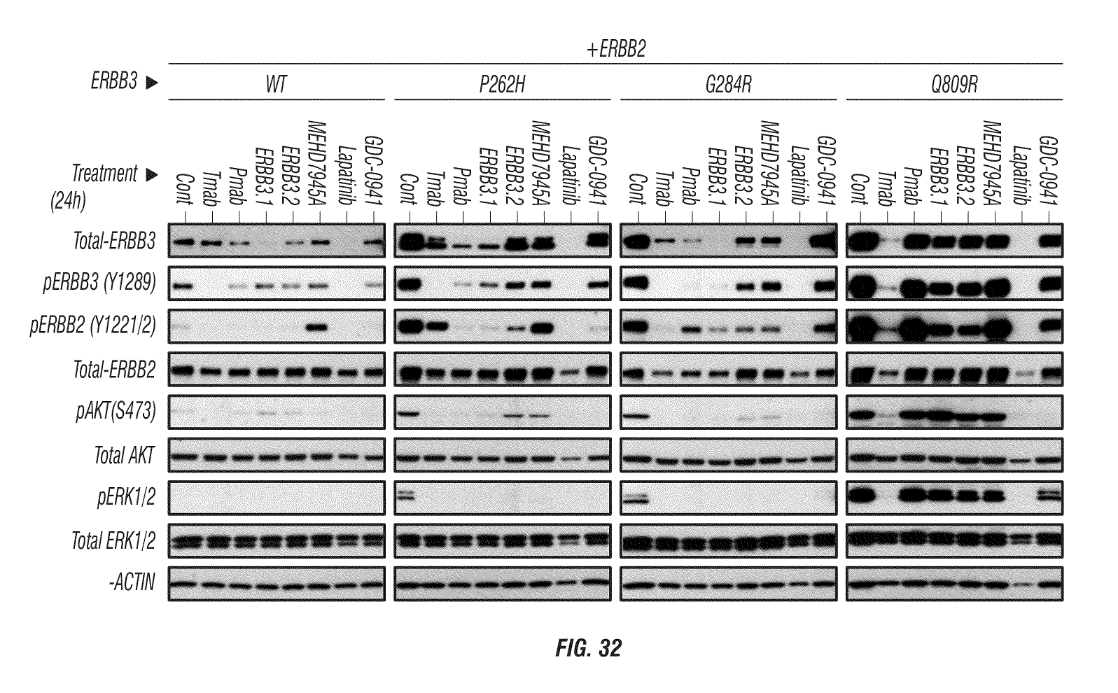

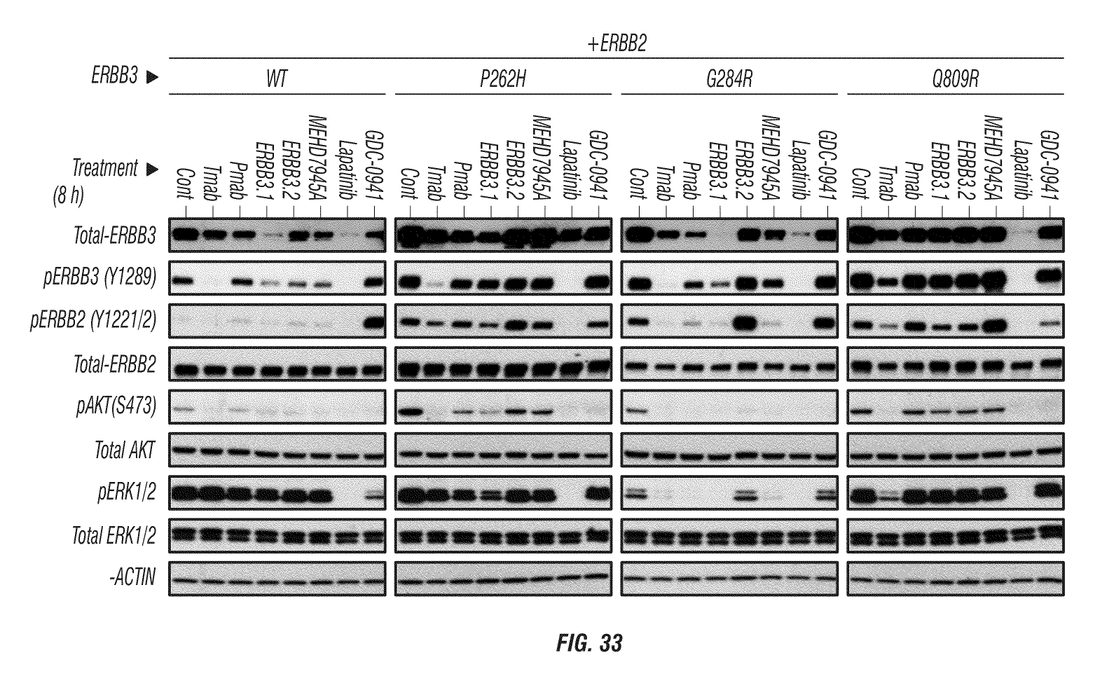

Multiple agents that target the ERBB receptors directly are approved for treating various cancers (Baselga and Swain Nature Reviews Cancer 9, 463-475 (2009); Alvarez et al. Journal of Clinical Oncology 28, 3366-3379 (2010)). Several additional candidate drugs that target ERBB family members, including ERBB3, and their downstream components are in various stages of clinical testing and development (Alvarez et al. Journal of Clinical Oncology 28, 3366-3379 (2010)). We tested trastuzumab—an anti-ERBB2 antibody that binds ERBB2 domain IV (Junttila et al. Cancer Cell 15, 429-440 (2009)), pertuzumab—an anti-ERBB2 antibody that binds ERBB2 domain II and prevents dimerization (Junttila et al. Cancer Cell 15, 429-440 (2009)), anti-ERBB3.1—an anti-ERBB3 that block ligand binding (binds domain III) (Schaefer, G. et al. Cancer Cell (2011)), anti-ERBB3.2—an anti-ERBB3 antibody, that bind domain III and blocks ligand binding (Wilson et al. Cancer Cell 20, 158-172 (2011)), MEHD7945A—a dual ERBB3/EGFR antibody that blocks ligand binding (binds domain III of EGFR and ERBB3) (Schaefer, G. et al. Cancer Cell (2011)), cetuximab—an EGFR antibody that blocks ligand binding (binds to domain III of EGFR) (Li, S. et al. Cancer Cell 7, 301-311 (2005)), Lapatinib (Medina, P. J. & Goodin, S. Clin Ther 30, 1426-1447 (2008))—a dual ERBB2/EGFR small molecule inhibitor and GDC-0941 (Edgar, K. A. et al. Cancer Research 70, 1164-1172 (2010))—a PI3K inhibitor, for their effect on blocking cell proliferation and colony formation using the BaF3 system (FIG. 28, FIG. 29 and FIG. 30). We also tested a subset of the antibodies for in vivo for efficacy (FIG. 31). We found that in both the proliferation and colony formation assays, the small molecular inhibitor lapatinib to be quite effective against all the mutants and GDC-0941 to be effective against all the mutants tested except against Q809R were it was only partially effective at the tested dose (FIGS. 28 and 29). Among the antibodies tested in the colony formation assay, trastuzumab anti-ERBB3.2 and MEHD7945A were all effective against all the mutants tested (FIGS. 28 and 29). However, pertuzumab, anti-ERBB3.1 and GDC-0941 though very effective in blocking proliferation and colony formation induced by ERBB3 ECD mutants, were only modestly effective against the Q809R kinase domain ERBB3 mutant (FIGS. 28 and 29). Consistent with this, in vitro in BaF3 cells co-expressing mutant ERBB3 and ERBB2, when efficacious, these agents, blocked or reduced pAKT and/or pERK levels, and also the levels of ERBB3 and/or pERBB3 (FIG. 32 and FIG. 33).

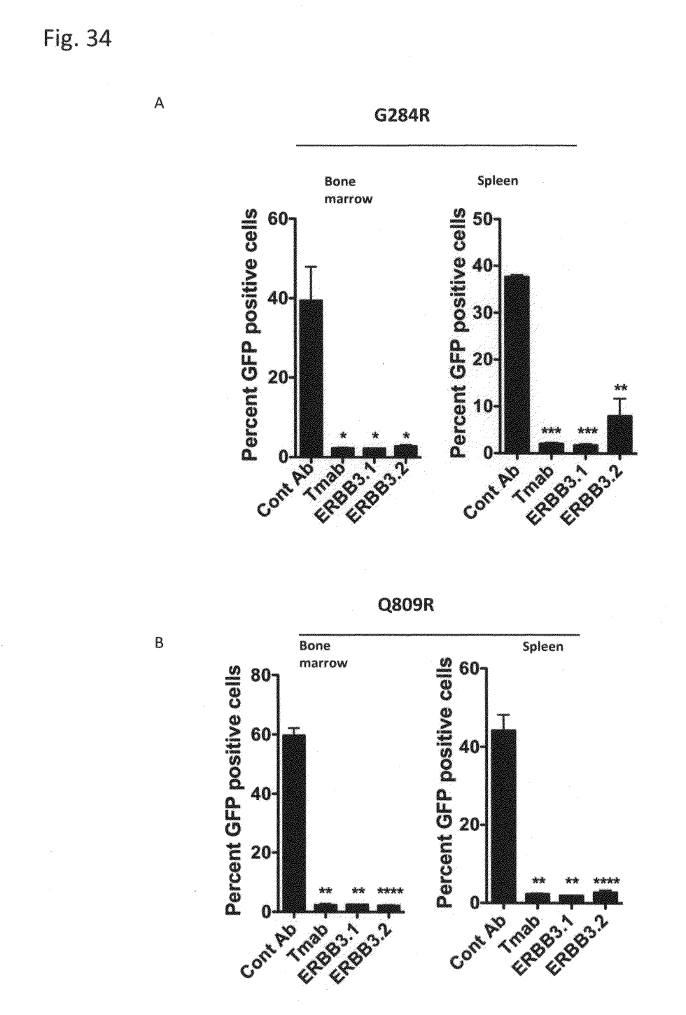

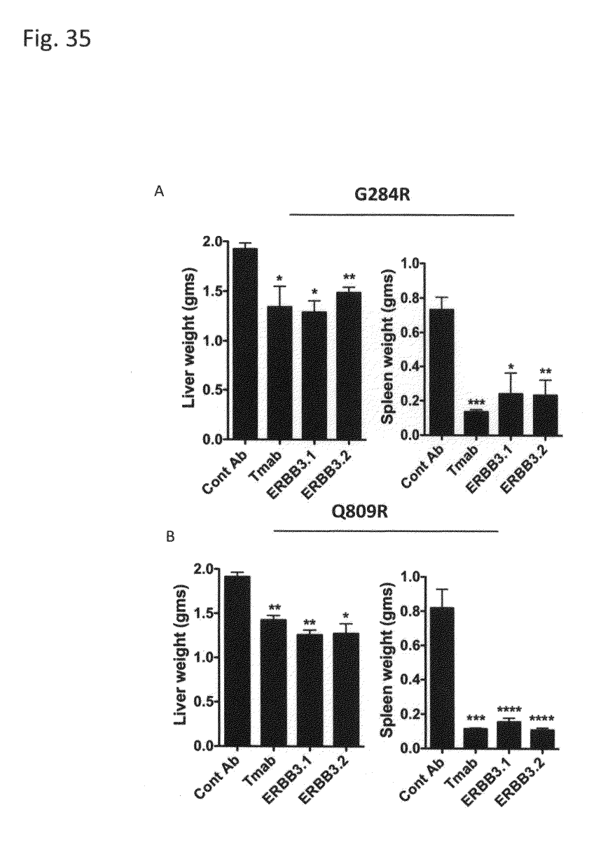

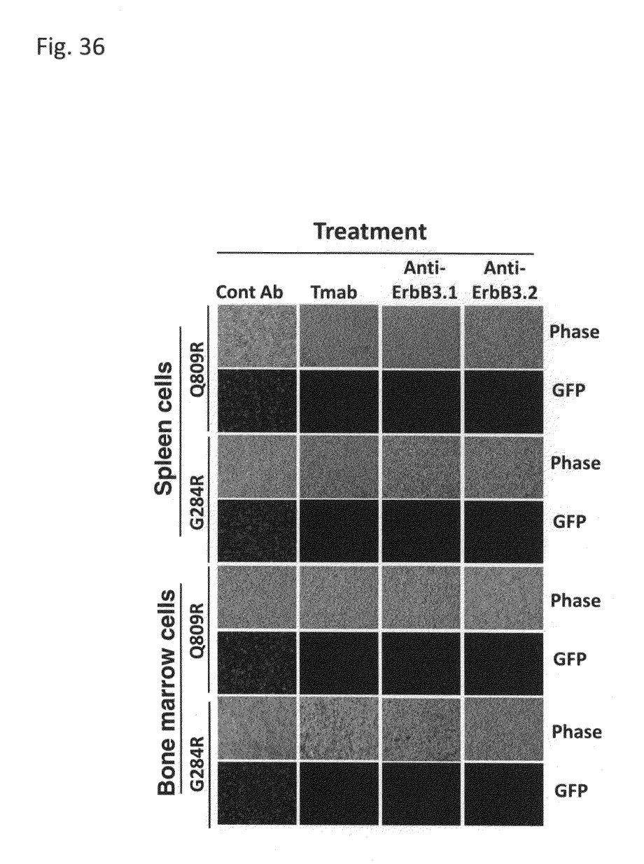

We also tested trastuzumab, anti-ERBB3.1 and anti-ERBB3.2 against G284R and Q809R ERBB3 mutants using the BaF3 system in vivo (FIGS. 31, 34 and 35). As observed in vitro, trastuzumab was very effective in blocking leukemia-like disease in mice receiving BaF3 expressing G284R or Q809R ERBB3/ERBB2 (FIG. 31A). Similarly, both anti-ERBB3.1 and anti-ERBB3.2 blocked the development of leukemia-like disease in mice receiving BaF3 co-expressing G284R ERBB3-ECD and ERBB2 (FIG. 31A). However, these anti-ERBB3 antibodies were only partially effective in blocking disease development in mice receiving BaF3 cells expressing Q809R ERBB3/ERBB2, although they significantly improved survival compared to untreated control animals (FIG. 31B). Consistent with the efficacy observed for the targeted therapeutics we found a significant decrease in infiltrating BaF3 cells expressing the ERBB3 mutants in the spleen and bone marrow (FIG. 34 and FIG. 36). Concomitant with the reduced infiltration of BaF3 cells observed, the spleen and liver weights were within the normal range expected for Balb/C nude mice (FIG. 35 and FIG. 25). These data indicate that multiple therapeutics, either in development or approved for human use, can be effective against ERBB3-mutant driven tumors.

In this study we report the identification of frequent ERBB3 somatic mutations in colon and gastric cancers. Several of the mutations we identified occur in multiple independent samples forming hotspots characteristic of oncogenic mutations.

These in vitro and in vivo functional studies demonstrate the oncogenic nature of both the ECD and kinase domain ERBB3 mutations. Further, using ligand titration experiments we show that some of the ECD mutants, V104M, P262H, Q284R and T389K, while oncogenic in the absence of ERBB3 ligand NRG1, can be further stimulated by addition of NRG1. ECD mutations may shift the equilibrium between tethered and untethered ERBB3 ECD towards an untethered confirmation relative to WT.

Having tested several therapeutic agents for their utility in targeting ERBB3-mutant driven oncogenic signaling both in vitro and in vivo, we found that multiple small molecule inhibitors, anti-ERBB2 and anti-ERBB3 ECD antibodies to be quite effective in blocking oncogenic signaling by a majority of the ERBB3 mutants tested. Interestingly, pertuzumab, anti-ERBB3.1 and GDC-0941 were not as effective in blocking the kinase domain mutant Q809R, indicating a distinct mode of action by this mutant. Previous studies have shown that while pertuzumab is quite effective in blocking ligand-mediated ERBB3/ERBB2 dimerization, trastuzumab is more effective in blocking ligand-independent ERBB2/ERBB3 dimer formation (Junttila, T. T. et al. Cancer Cell 15, 429-440 (2009)). Consistent with this, the ligand non-responsive kinase domain ERBB3 mutant Q809R is much more responsive to inhibition by trastuzumab compared to pertuzumab suggesting a potential role for a non-liganded heterodimeric complex in Q809R ERBB3 signaling. Although the PI3K inhibitor GDC-0941 is quite active against most of the ERBB3 mutants tested, its reduced efficacy in blocking kinase domain mutant Q809R, suggest the engagement of other downstream signaling molecules, besides the PI3Kinase.

shRNA-Mediated ERBB3-Knock-Down Affects In Vivo Growth

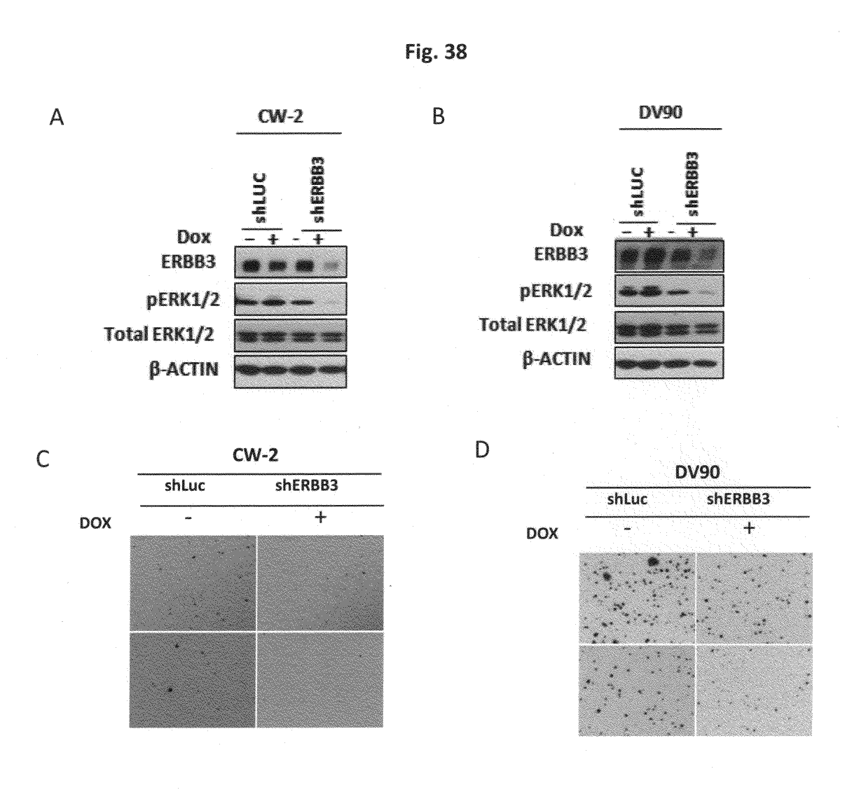

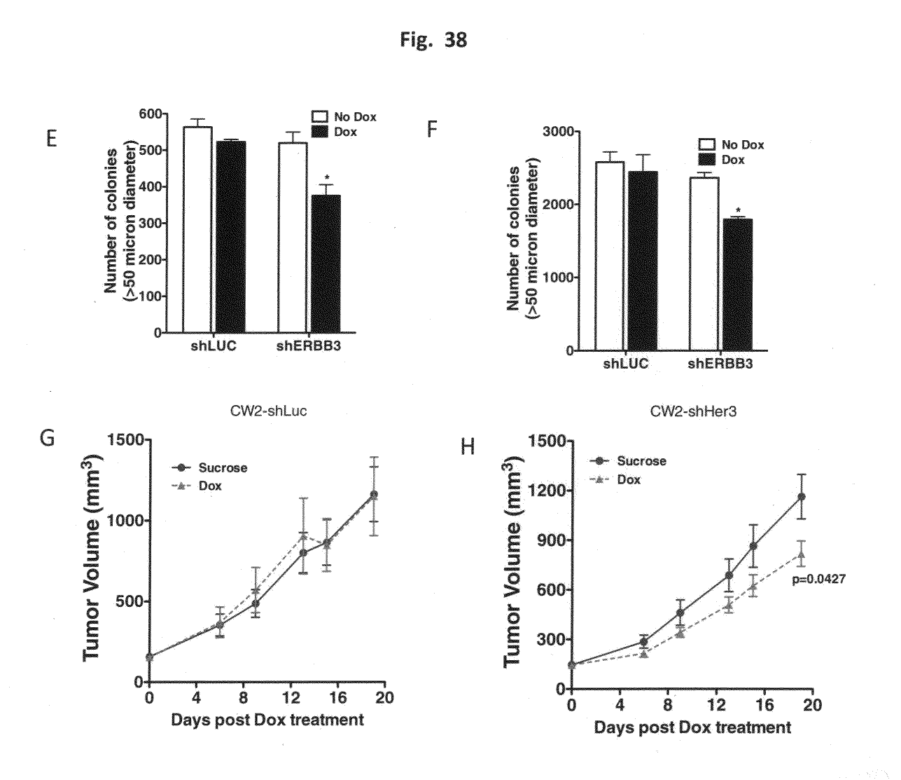

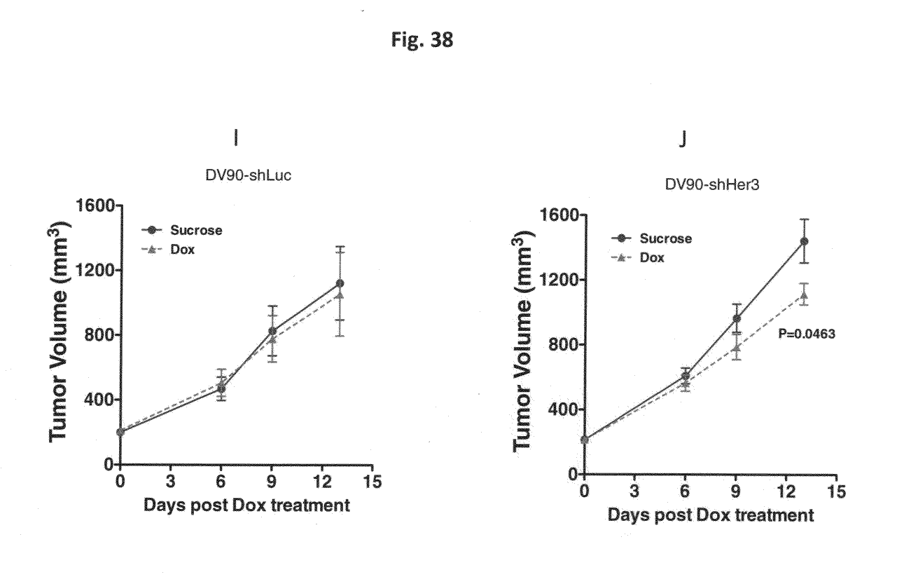

Having established the oncogenic activity of ERBB3 mutants in IMCE cells, we sought to test the effect of knocking down ERBB3 in tumor cell lines. A recent study reported CW-2, a colon cell line, and DV90, a lung line, that express ERBB3 E928G and V104M mutants, respectively. We generated stable CW-2 and DV90 cell lines that express a doxycycline (dox)-inducible shRNA that targets ERBB3 using a previously published targeting constructs (Garnett et al. (2012) Nature 483, 570-575). We also generated control lines that expressed an dox-inducible luciferace (luc) targeting sequencing. Upon dox-induction, in contrast to the luc shRNA expressing lines, levels of ERBB3 and pERK was decreased in cells that expressed the ERBB3 shRNA (FIG. 38A-B). Consistent with the loss of ERBB3 following dox-induction both DV90 and CW-2 showed reduced anchorage independent growth compared to luciferase shRNA lines or uninduced lines (FIG. 38C-F). We next tested whether knockdown of ERBB3 in DV90 and CW-2 cells might affect their ability to form tumors in vivo. Upon dox-mediated induction of ERBB3 targeting shRNA, we found that both DV90 and CW-2 cells showed a significantly decrease in tumor growth compared to animals bearing DV90 or CW-2 cell that expressed luc-shRNA or were not induced to express the ERBB3 shRNA (FIG. 38G-J). These data taken together further confirm the role of ERBB3 mutations in tumorigenesis.

What is claimed is:

1. An ErbB3 gastrointestinal cancer detecting agent comprising a reagent capable of specifically binding to an ErbB3 mutation in an ErbB3 nucleic acid sequence.

2. The cancer detecting agent of claim 1, wherein the ErbB3 nucleic acid sequence comprises SEQ ID NO:3 or 1.

3. The cancer detecting agent of claim 1, wherein the reagent comprises a polynucleotide of formula
5′Xₐ—Y—Z_{b}3′ Formula I,
wherein
X is any nucleic acid and a is between about 0 and about 250;
Y is an ErbB3 mutation codon; and
Z is any nucleic acid and b is between about 0 and about 250.

4. The cancer detecting agent of claim 3, wherein the mutation codon encodes (i) an amino acid at a position of SEQ ID NO:2 selected from the group consisting of 104, 809, 232, 262, 284, 325, 846, 928, 60, 111, 135, 295, 406, 453, 498, 1089, and 1164; or (ii) a stop codon at position 193.

5. A method of determining the presence of ErbB3 gastrointestinal cancer in a subject comprising detecting in a biological sample obtained from the subject a mutation in a nucleic acid sequence encoding ErbB3, wherein the mutation results in an amino acid change at at least one position of the ErbB3 amino acid sequence and wherein the mutation is indicative of an ErbB3 gastrointestinal cancer in the subject.

6. The method of claim 5, wherein the mutation resulting in an amino acid change is at a position of SEQ ID NO:2 selected from the group consisting of 104, 809, 232, 262, 284, 325, 846, 928, 60, 111, 135, 295, 406, 453, 498, 1089, 1164, and 193.

7. A method of determining the presence of ErbB3 cancer in a subject comprising detecting in a biological sample obtained from the subject the presence or absence of an amino acid mutation in a nucleic acid sequence encoding ErbB3, wherein the mutation results in an amino acid change at at least one position in SEQ ID NO: 2 selected from the group consisting of 104, 809, 232, 262, 284, 325, 846, 928, 60, 111, 135, 295, 406, 453, 498, 1089, 1164, 193, 492, and 714, and wherein the presence of the mutation is indicative of an ErbB3 cancer in the subject.

8. The method of claim 5 or 7, further comprising administering a therapeutic agent to said subject.

9. The method of claim 8, wherein the therapeutic agent is an ErbB inhibitor.

10. The method of claim 9, wherein the ErbB inhibitor is selected from the group consisting of an EGFR antagonist, an ErbB2 antagonist, an ErbB3 antagonist, an ErbB4 antagonist, and an EGFR/ErbB3 antagonist.

11. The method of claim 10, wherein the inhibitor is a small molecule inhibitor.

12. The method of claim 10, wherein the antagonist is an antagonist antibody.

13. The method of claim 12, wherein the antibody is selected from the group consisting of a monoclonal antibody, a bispecific antibody, a chimeric antibody, a human antibody, a humanized antibody and an antibody fragment.

14. The detecting agent of claim 1 or the method of claim 5, wherein the gastrointestinal cancer is gastric cancer or colon cancer.

15. The method of claim 7, wherein the ErbB3 cancer is selected from the group consisting of gastric, colon, esophageal, rectal, cecum, non-small-cell lung (NSCLC) adenocarinoma, NSCLC (Squamous carcinoma), renal carcinoma, melanoma, ovarian, lung large cell, small-cell lung cancer (SCLC), hepatocellular (HCC), lung, and pancreatic.

16. The method of claim 5 or 7, further comprising (i) identifying the subject in need and/or (ii) obtaining the sample from a subject in need.

17. The method of claim 5 or 7, wherein the detecting comprises amplifying or sequencing the mutation and detecting the mutation or sequence thereof.

18. The method of claim 17, wherein the amplifying comprises admixing an amplification primer or amplification primer pair with a nucleic acid template isolated from the sample.

19. The method of claim 18, wherein the primer or primer pair is complementary or partially complementary to a region proximal to or including said mutation, and is capable of initiating nucleic acid polymerization by a polymerase on the nucleic acid template.

20. The method of claim 18, further comprising extending the primer or primer pair in a DNA polymerization reaction comprising a polymerase and the template nucleic acid to generate an amplicon.

21. The method of claim 17, wherein the mutation is detected by a process that includes one or more of: sequencing the mutation in a genomic DNA isolated from the biological sample, hybridizing the mutation or an amplicon thereof to an array, digesting the mutation or an amplicon thereof with a restriction enzyme, or real-time PCR amplification of the mutation.

22. The method of claim 17, comprising partially or fully sequencing the mutation in a nucleic acid isolated from the biological sample.

23. The method of claim 17, wherein the amplifying comprises performing a polymerase chain reaction (PCR), reverse transcriptase PCR (RT-PCR), or ligase chain reaction (LCR) using a nucleic acid isolated from the biological sample as a template in the PCR, RT-PCR, or LCR.

24. A method of treating gastrointestinal cancer in a subject in need comprising
a) detecting in a biological sample obtained from the subject a mutation in a nucleic acid sequence encoding ErbB3, wherein the mutation results in an amino acid change at at least one position of the ErbB3 amino acid sequence and wherein the mutation is indicative of an ErbB3 gastrointestinal cancer in the subject; and
b) administering a therapeutic agent to said subject.

25. The method of claim 24, wherein the mutation resulting in an amino acid change is at a position of SEQ ID NO:2 selected from the group consisting of 104, 809, 232, 262, 284, 325, 846, 928, 60, 111, 135, 295, 406, 453, 498, 1089, 1164, and 193.

26. A method of treating an ErbB3 cancer in a subject comprising:
a) detecting in a biological sample obtained from the subject the presence or absence of an amino acid mutation in a nucleic acid sequence encoding ErbB3, wherein the mutation results in an amino acid change at at least one position in SEQ ID NO: 2 selected from the group consisting of 104, 809, 232, 262, 284, 325, 846, 928, 60, 111, 135, 295, 406, 453, 498, 1089, 1164, 193, 492, and 714, and wherein the presence of the mutation is indicative of an ErbB3 cancer in the subject; and
b) administering a therapeutic agent to said subject.

27. The method of claim 24 or 26, wherein the therapeutic agent is an ErbB inhibitor.

28. The method of claim 27, wherein the ErbB inhibitor is selected from the group consisting of an EGFR antagonist, an ErbB2 antagonist, an ErbB3 antagonist, an ErbB4 antagonist, and an EGFR/ErbB3 antagonist.

29. The method of claim 28, wherein the antagonist is a small molecule inhibitor.

30. The method of claim 28, wherein the antagonist is an antagonist antibody.

31. The method of claim 30, wherein the antibody is selected from the group consisting of a monoclonal antibody, a bispecific antibody, a chimeric antibody, a human antibody, a humanized antibody and an antibody fragment.

32. The method of claim 24, wherein the gastrointestinal cancer is gastric cancer or colon cancer.

33. The method of claim 26, wherein the ErbB3 cancer is selected from the group consisting of gastric, colon, esophageal, rectal, cecum, colorectal, non-small-cell lung (NSCLC) adenocarinoma, NSCLC (Squamous carcinoma), renal carcinoma, melanoma, ovarian, lung large cell, small-cell lung cancer (SCLC), hepatocellular (HCC), lung, and pancreatic.


SEQUENCE LISTING (231 sequences)

SEQ ID NO: 1 - protein, 1342 aa, Homo sapiens
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 2 - DNA, 5765 bp, Homo sapiens
actccagcctcgcgcgggagggggcgcggccgtgactcacccccttccctctgcgttcctccctccctctctctctctctctcacacacacacacccctcccctgccatccctccccggactccggctccggctccgattgcaatttgcaacctccgctgccgtcgccgcagcagccaccaattcgccagcggttcaggtggctcttgcctcgatgtcctagcctaggggcccccgggccggacttggctgggctcccttcaccctctgcggagtcatgagggcgaacgacgctctgcaggtgctgggcttgcttttcagcctggcccggggctccgaggtgggcaactctcaggcagtgtgtcctgggactctgaatggcctgagtgtgaccggcgatgctgagaaccaataccagacactgtacaagctctacgagaggtgtgaggtggtgatggggaaccttgagattgtgctcacgggacacaatgccgacctctccttcctgcagtggattcgagaagtgacaggctatgtcctcgtggccatgaatgaattctctactctaccattgcccaacctccgcgtggtgcgagggacccaggtctacgatgggaagtttgccatcttcgtcatgttgaactataacaccaactccagccacgctctgcgccagctccgcttgactcagctcaccgagattctgtcagggggtgtttatattgagaagaacgataagctttgtcacatggacacaattgactggagggacatcgtgagggaccgagatgctgagatagtggtgaaggacaatggcagaagctgtcccccctgtcatgaggtttgcaaggggcgatgctggggtcctggatcagaagactgccagacattgaccaagaccatctgtgctcctcagtgtaatggtcactgctttgggcccaaccccaaccagtgctgccatgatgagtgtgccgggggctgctcaggccctcaggacacagactgctttgcctgccggcacttcaatgacagtggagcctgtgtacctcgctgtccacagcctcttgtctacaacaagctaactttccagctggaacccaatccccacaccaagtatcagtatggaggagtttgtgtagccagctgtccccataactttgtggtggatcaaacatcctgtgtcagggcctgtcctcctgacaagatggaagtagataaaaatgggctcaagatgtgtgagccttgtgggggactatgtcccaaagcctgtgagggaacaggctctgggagccgcttccagactgtggactcgagcaacattgatggatttgtgaactgcaccaagatcctgggcaacctggactttctgatcaccggcctcaatggagacccctggcacaagatccctgccctggacccagagaagctcaatgtcttccggacagtacgggagatcacaggttacctgaacatccagtcctggccgccccacatgcacaacttcagtgttttttccaatttgacaaccattggaggcagaagcctctacaaccggggcttctcattgttgatcatgaagaacttgaatgtcacatctctgggcttccgatccctgaaggaaattagtgctgggcgtatctatataagtgccaataggcagctctgctaccaccactctttgaactggaccaaggtgcttcgggggcctacggaagagcgactagacatcaagcataatcggccgcgcagagactgcgtggcagagggcaaagtgtgtgacccactgtgctcctctgggggatgctggggcccaggccctggtcagtgcttgtcctgtcgaaattatagccgaggaggtgtctgtgtgacccactgcaactttctgaatggggagcctcgagaatttgcccatgaggccgaatgcttctcctgccacccggaatgccaacccatggagggcactgccacatgcaatggctcgggctctgatacttgtgctcaatgtgcccattttcgagatgggccccactgtgtgagcagctgcccccatggagtcctaggtgccaagggcccaatctacaagtacccagatgttcagaatgaatgtcggccctgccatgagaactgcacccaggggtgtaaaggaccagagcttcaagactgtttaggacaaacactggtgctgatcggcaaaacccatctgacaatggctttgacagtgatagcaggattggtagtgattttcatgatgctgggcggcacttttctctactggcgtgggcgccggattcagaataaaagggctatgaggcgatacttggaacggggtgagagcatagagcctctggaccccagtgagaaggctaacaaagtcttggccagaatcttcaaagagacagagctaaggaagcttaaagtgcttggctcgggtgtctttggaactgtgcacaaaggagtgtggatccctgagggtgaatcaatcaagattccagtctgcattaaagtcattgaggacaagagtggacggcagagttttcaagctgtgacagatcatatgctggccattggcagcctggaccatgcccacattgtaaggctgctgggactatgcccagggtcatctctgcagcttgtcactcaatatttgcctctgggttctctgctggatcatgtgagacaacaccggggggcactggggccacagctgctgctcaactggggagtacaaattgccaagggaatgtactaccttgaggaacatggtatggtgcatagaaacctggctgcccgaaacgtgctactcaagtcacccagtcaggttcaggtggcagattttggtgtggctgacctgctgcctcctgatgataagcagctgctatacagtgaggccaagactccaattaagtggatggcccttgagagtatccactttgggaaatacacacaccagagtgatgtctggagctatggtgtgacagtttgggagttgatgaccttcggggcagagccctatgcagggctacgattggctgaagtaccagacctgctagagaagggggagcggttggcacagccccagatctgcacaattgatgtctacatggtgatggtcaagtgttggatgattgatgagaacattcgcccaacctttaaagaactagccaatgagttcaccaggatggcccgagacccaccacggtatctggtcataaagagagagagtgggcctggaatagcccctgggccagagccccatggtctgacaaacaagaagctagaggaagtagagctggagccagaactagacctagacctagacttggaagcagaggaggacaacctggcaaccaccacactgggctccgccctcagcctaccagttggaacacttaatcggccacgtgggagccagagccttttaagtccatcatctggatacatgcccatgaaccagggtaatcttggggagtcttgccaggagtctgcagtttctgggagcagtgaacggtgcccccgtccagtctctctacacccaatgccacggggatgcctggcatcagagtcatcagaggggcatgtaacaggctctgaggctgagctccaggagaaagtgtcaatgtgtaggagccggagcaggagccggagcccacggccacgcggagatagcgcctaccattcccagcgccacagtctgctgactcctgttaccccactctccccacccgggttagaggaagaggatgtcaacggttatgtcatgccagatacacacctcaaaggtactccctcctcccgggaaggcaccctttcttcagtgggtctcagttctgtcctgggtactgaagaagaagatgaagatgaggagtatgaatacatgaaccggaggagaaggcacagtccacctcatccccctaggccaagttcccttgaggagctgggttatgagtacatggatgtggggtcagacctcagtgcctctctgggcagcacacagagttgcccactccaccctgtacccatcatgcccactgcaggcacaactccagatgaagactatgaatatatgaatcggcaacgagatggaggtggtcctgggggtgattatgcagccatgggggcctgcccagcatctgagcaagggtatgaagagatgagagcttttcaggggcctggacatcaggccccccatgtccattatgcccgcctaaaaactctacgtagcttagaggctacagactctgcctttgataaccctgattactggcatagcaggcttttccccaaggctaatgcccagagaacgtaactcctgctccctgtggcactcagggagcatttaatggcagctagtgcctttagagggtaccgtcttctccctattccctctctctcccaggtcccagccccttttccccagtcccagacaattccattcaatctttggaggcttttaaacattttgacacaaaattcttatggtatgtagccagctgtgcactttcttctctttcccaaccccaggaaaggttttccttattttgtgtgctttcccagtcccattcctcagcttcttcacaggcactcctggagatatgaaggattactctccatatcccttcctctcaggctcttgactacttggaactaggctcttatgtgtgcctttgtttcccatcagactgtcaagaagaggaaagggaggaaacctagcagaggaaagtgtaattttggtttatgactcttaaccccctagaaagacagaagcttaaaatctgtgaagaaagaggttaggagtagatattgattactatcataattcagcacttaactatgagccaggcatcatactaaacttcacctacattatctcacttagtcctttatcatccttaaaacaattctgtgacatacatattatctcattttacacaaagggaagtcgggcatggtggctcatgcctgtaatctcagcactttgggaggctgaggcagaaggattacctgaggcaaggagtttgagaccagcttagccaacatagtaagacccccatctctttaaaaaaaaaaaaaaaaaaaaaaaaaaaactttagaactgggtgcagtggctcatgcctgtaatcccagccagcactttgggaggctgagatgggaagatcacttgagcccagaattagagataagcctatggaaacatagcaagacactgtctctacaggggaaaaaaaaaaaagaaactgagccttaaagagatgaaataaattaagcagtagatccaggatgcaaaatcctcccaattcctgtgcatgtgctcttattgtaaggtgccaagaaaaactgatttaagttacagcccttgtttaaggggcactgtttcttgtttttgcactgaatcaagtctaaccccaacagccacatcctcctatacctagacatctcatctcaggaagtggtggtgggggtagtcagaaggaaaaataactggacatctttgtgtaaaccataatccacatgtgccgtaaatgatcttcactccttatccgagggcaaattcacaaggatccccaagatccacttttagaagccattctcatccagcagtgagaagcttccaggtaggacagaaaaaagatccagcttcagctgcacacctctgtccccttggatggggaactaagggaaaacgtctgttgtatcactgaagttttttgttttgtttttatacgtgtctgaataaaaatgccaaagttttttttcagcaaaaaaaa

SEQ ID NO: 3 - DNA, 13 bp, Artificial Sequence
  source: /note=“Description of Artificial Sequence Synthetic primer”
tcccctgccatcc

SEQ ID NO: 4 - DNA, 15 bp, Artificial Sequence
  source: /note=“Description of Artificial Sequence Synthetic primer”
ggccactacagcttc

SEQ ID NO: 5 - DNA, 16 bp, Artificial Sequence
  source: /note=“Description of Artificial Sequence Synthetic primer”
gcgtaactccgtctca

SEQ ID NO: 6 - DNA, 19 bp, Artificial Sequence
  source: /note=“Description of Artificial Sequence Synthetic primer”
ctcctcatcttataaaggg

SEQ ID NO: 7 - DNA, 15 bp, Artificial Sequence
  source: /note=“Description of Artificial Sequence Synthetic primer”
cgccccttgttgaca

SEQ ID NO: 8 - DNA, 17 bp, Artificial Sequence
  source: /note=“Description of Artificial Sequence Synthetic primer”
atcagaagactgccaga

SEQ ID NO: 9 - DNA, 16 bp, Artificial Sequence
  source: /note=“Description of Artificial Sequence Synthetic primer”
ccagtgctgccatgat

SEQ ID NO: 10 - DNA, 24 bp, Artificial Sequence
  source: /note=“Description of Artificial Sequence Synthetic primer”
caaatagtgaagagacttttgaat

SEQ ID NO: 11 - DNA, 17 bp, Artificial Sequence
  source: /note=“Description of Artificial Sequence Synthetic primer”
ctgtcctcctgacaaga

SEQ ID NO: 12 - DNA, 19 bp, Artificial Sequence
  source: /note=“Description of Artificial Sequence Synthetic primer”
cttgtttgcacaagatgct

SEQ ID NO: 13 - DNA, 15 bp, Artificial Sequence
  source: /note=“Description of Artificial Sequence Synthetic primer”
tcacaggtgagtggc

SEQ ID NO: 14 - DNA, 19 bp, Artificial Sequence
  source: /note=“Description of Artificial Sequence Synthetic primer”
cctcaaaaccaaagggttt

SEQ ID NO: 15 - DNA, 15 bp, Artificial Sequence
  source: /note=“Description of Artificial Sequence Synthetic primer”
agggtctgctaggtg

SEQ ID NO: 16 - DNA, 16 bp, Artificial Sequence
  source: /note=“Description of Artificial Sequence Synthetic primer”
cagtcaaggatgggtg

SEQ ID NO: 17 - DNA, 15 bp, Artificial Sequence
  source: /note=“Description of Artificial Sequence Synthetic primer”
tggagcatctgggga

SEQ ID NO: 18 - DNA, 19 bp, Artificial Sequence
  source: /note=“Description of Artificial Sequence Synthetic primer”
tcaagggagtttcacagaa

SEQ ID NO: 19 - DNA, 20 bp, Artificial Sequence
  source: /note=“Description of Artificial Sequence Synthetic primer”
ctttcagtagtctaagactg

SEQ ID NO: 20 - DNA, 15 bp, Artificial Sequence
  source: /note=“Description of Artificial Sequence Synthetic primer”
cagggtctgtacctc

SEQ ID NO: 21 - DNA, 18 bp, Artificial Sequence
  source: /note=“Description of Artificial Sequence Synthetic primer”
gaagcttaaagtgcttgg

SEQ ID NO: 22 - DNA, 19 bp, Artificial Sequence
  source: /note=“Description of Artificial Sequence Synthetic primer”
ggagagaggacaatattag

SEQ ID NO: 23 - DNA, 16 bp, Artificial Sequence
  source: /note=“Description of Artificial Sequence Synthetic primer”
cccaaaaccaaccctc

SEQ ID NO: 24 - DNA, 15 bp, Artificial Sequence
  source: /note=“Description of Artificial Sequence Synthetic primer”
agagcgagactccgt

SEQ ID NO: 25 - DNA, 15 bp, Artificial Sequence
  source: /note=“Description of Artificial Sequence Synthetic primer”
gatgccctctctacc

SEQ ID NO: 26 - DNA, 19 bp, Artificial Sequence
  source: /note=“Description of Artificial Sequence Synthetic primer”
agatggggtttcactatgt

SEQ ID NO: 27 - DNA, 18 bp, Artificial Sequence
  source: /note=“Description of Artificial Sequence Synthetic primer”
gcccaacctttaaagaac

SEQ ID NO: 28 - DNA, 16 bp, Artificial Sequence
  source: /note=“Description of Artificial Sequence Synthetic primer”
gcctaccagttggaac

SEQ ID NO: 29 - DNA, 16 bp, Artificial Sequence
  source: /note=“Description of Artificial Sequence Synthetic primer”
ggcagtgaacaaccca

SEQ ID NO: 30 - DNA, 17 bp, Artificial Sequence
  source: /note=“Description of Artificial Sequence Synthetic primer”
cgtccagtctctctaca

SEQ ID NO: 31 - DNA, 16 bp, Artificial Sequence
  source: /note=“Description of Artificial Sequence Synthetic primer”
ctcaaaggtgcctgac

SEQ ID NO: 32 - DNA, 16 bp, Artificial Sequence
  source: /note=“Description of Artificial Sequence Synthetic primer”
cttgaggagctgggtt

SEQ ID NO: 33 - DNA, 13 bp, Artificial Sequence
  source: /note=“Description of Artificial Sequence Synthetic primer”
cccgagcctgacc

SEQ ID NO: 34 - DNA, 15 bp, Artificial Sequence
  source: /note=“Description of Artificial Sequence Synthetic primer”
tcccagatgacagcc

SEQ ID NO: 35 - DNA, 17 bp, Artificial Sequence
  source: /note=“Description of Artificial Sequence Synthetic primer”
ggccctctattgcttag

SEQ ID NO: 36 - DNA, 19 bp, Artificial Sequence
  source: /note=“Description of Artificial Sequence Synthetic primer”
tggtttagattccaggaga

SEQ ID NO: 37 - DNA, 17 bp, Artificial Sequence
  source: /note=“Description of Artificial Sequence Synthetic primer”
cactgaggagcacagat

SEQ ID NO: 38 - DNA, 15 bp, Artificial Sequence
  source: /note=“Description of Artificial Sequence Synthetic primer”
tgtggacagcgaggt

SEQ ID NO: 39 - DNA, 15 bp, Artificial Sequence
  source: /note=“Description of Artificial Sequence Synthetic primer”
ggaggactggacgta

SEQ ID NO: 40 - DNA, 19 bp, Artificial Sequence
  source: /note=“Description of Artificial Sequence Synthetic primer”
atcttggtgcagttcacaa

SEQ ID NO: 41 - DNA, 19 bp, Artificial Sequence
  source: /note=“Description of Artificial Sequence Synthetic primer”
atggaggatgtgttaagca

SEQ ID NO: 42 - DNA, 17 bp, Artificial Sequence
  source: /note=“Description of Artificial Sequence Synthetic primer”
gactggatgttcaggta

SEQ ID NO: 43 - DNA, 15 bp, Artificial Sequence
  source: /note=“Description of Artificial Sequence Synthetic primer”
gatccactgagaggg

SEQ ID NO: 44 - DNA, 15 bp, Artificial Sequence
  source: /note=“Description of Artificial Sequence Synthetic primer”
aggactcccagcaag

SEQ ID NO: 45 - DNA, 16 bp, Artificial Sequence
  source: /note=“Description of Artificial Sequence Synthetic primer”
ccaagtcctgaccttc

SEQ ID NO: 46 - DNA, 19 bp, Artificial Sequence
  source: /note=“Description of Artificial Sequence Synthetic primer”
tcccaaggtcaattccata

SEQ ID NO: 47 - DNA, 13 bp, Artificial Sequence
  source: /note=“Description of Artificial Sequence Synthetic primer”
cacccacctcggc

SEQ ID NO: 48 - DNA, 20 bp, Artificial Sequence
  source: /note=“Description of Artificial Sequence Synthetic primer”
cagtcttagactactgaaag

SEQ ID NO: 49 - DNA, 18 bp, Artificial Sequence
  source: /note=“Description of Artificial Sequence Synthetic primer”
accacactacttccttga

SEQ ID NO: 50 - DNA, 19 bp, Artificial Sequence
  source: /note=“Description of Artificial Sequence Synthetic primer”
tgcagactggaatcttgat

SEQ ID NO: 51 - DNA, 18 bp, Artificial Sequence
  source: /note=“Description of Artificial Sequence Synthetic primer”
gaaaccaacaggttcaca

SEQ ID NO: 52 - DNA, 15 bp, Artificial Sequence
  source: /note=“Description of Artificial Sequence Synthetic primer”
cgctcacatgctctg

SEQ ID NO: 53 - DNA, 17 bp, Artificial Sequence
  source: /note=“Description of Artificial Sequence Synthetic primer”
ccagtcccaagttcttg

SEQ ID NO: 54 - DNA, 16 bp, Artificial Sequence
  source: /note=“Description of Artificial Sequence Synthetic primer”
ctgtcacacctgttgc

SEQ ID NO: 55 - DNA, 16 bp, Artificial Sequence
  source: /note=“Description of Artificial Sequence Synthetic primer”
cagcctgggtgacaat

SEQ ID NO: 56 - DNA, 18 bp, Artificial Sequence
  source: /note=“Description of Artificial Sequence Synthetic primer”
ctctacttcctctagctt

SEQ ID NO: 57 - DNA, 19 bp, Artificial Sequence
  source: /note=“Description of Artificial Sequence Synthetic primer”
tgatggacttaaaaggctc

SEQ ID NO: 58 - DNA, 16 bp, Artificial Sequence
  source: /note=“Description of Artificial Sequence Synthetic primer”
cctcaggtgatccact

SEQ ID NO: 59 - DNA, 18 bp, Artificial Sequence
  source: /note=“Description of Artificial Sequence Synthetic primer”
ataaccgttgacatcctc

SEQ ID NO: 60 - DNA, 15 bp, Artificial Sequence
  source: /note=“Description of Artificial Sequence Synthetic primer”
gaggagggagtacct

SEQ ID NO: 61 - DNA, 16 bp, Artificial Sequence
  source: /note=“Description of Artificial Sequence Synthetic primer”
cccctgaaaagctctc

SEQ ID NO: 62 - DNA, 22 bp, Artificial Sequence
  source: /note=“Description of Artificial Sequence Synthetic primer”
gtcaaaatgtttaaaagcctcc

SEQ ID NO: 63 - DNA, 13 bp, Artificial Sequence
  source: /note=“Description of Artificial Sequence Synthetic primer”
cgcggccgtgact

SEQ ID NO: 64 - DNA, 18 bp, Artificial Sequence
  source: /note=“Description of Artificial Sequence Synthetic primer”
agaagagagaaagctctc

SEQ ID NO: 65 - DNA, 19 bp, Artificial Sequence
  source: /note=“Description of Artificial Sequence Synthetic primer”
agatcgcactattgtactc

SEQ ID NO: 66 - DNA, 16 bp, Artificial Sequence
  source: /note=“Description of Artificial Sequence Synthetic primer”
ctggacaggtgactga

SEQ ID NO: 67 - DNA, 15 bp, Artificial Sequence
  source: /note=“Description of Artificial Sequence Synthetic primer”
ctgggttgggactag

SEQ ID NO: 68 - DNA, 15 bp, Artificial Sequence
  source: /note=“Description of Artificial Sequence Synthetic primer”
ttgcaaggggcgatg

SEQ ID NO: 69 - DNA, 17 bp, Artificial Sequence
  source: /note=“Description of Artificial Sequence Synthetic primer”
tgtgctcctcagtgtaa

SEQ ID NO: 70 - DNA, 19 bp, Artificial Sequence
  source: /note=“Description of Artificial Sequence Synthetic primer”
cttacttctgctccttgta

SEQ ID NO: 71 - DNA, 18 bp, Artificial Sequence
  source: /note=“Description of Artificial Sequence Synthetic primer”
gatcaaacatcctgtgtc

SEQ ID NO: 72 - DNA, 21 bp, Artificial Sequence
  source: /note=“Description of Artificial Sequence Synthetic primer”
cccttaattctttgagtcttg

SEQ ID NO: 73 - DNA, 16 bp, Artificial Sequence
  source: /note=“Description of Artificial Sequence Synthetic primer”
gtcttccggacagtac

SEQ ID NO: 74 - DNA, 17 bp, Artificial Sequence
  source: /note=“Description of Artificial Sequence Synthetic primer”
cactgtctcatacagca

SEQ ID NO: 75 - DNA, 15 bp, Artificial Sequence
  source: /note=“Description of Artificial Sequence Synthetic primer”
cagagactgcggtga

SEQ ID NO: 76 - DNA, 20 bp, Artificial Sequence
  source: /note=“Description of Artificial Sequence Synthetic primer”
ctttctgaatgggtacagta

SEQ ID NO: 77 - DNA, 16 bp, Artificial Sequence
  source: /note=“Description of Artificial Sequence Synthetic primer”
gatctccaagggagac

SEQ ID NO: 78 - DNA, 18 bp, Artificial Sequence
  source: /note=“Description of Artificial Sequence Synthetic primer”
gaacctggaataacctca

SEQ ID NO: 79 - DNA, 15 bp, Artificial Sequence
  source: /note=“Description of Artificial Sequence Synthetic primer”
gcttctggacttccc

SEQ ID NO: 80 - DNA, 21 bp, Artificial Sequence
  source: /note=“Description of Artificial Sequence Synthetic primer”
gcacaaataacttcctcagtt

SEQ ID NO: 81 - DNA, 20 bp, Artificial Sequence
  source: /note=“Description of Artificial Sequence Synthetic primer”
cttcaaagagacagagctaa

SEQ ID NO: 82 - DNA, 19 bp, Artificial Sequence
  source: /note=“Description of Artificial Sequence Synthetic primer”
aaggaaattctgtatgccg

SEQ ID NO: 83 - DNA, 17 bp, Artificial Sequence
  source: /note=“Description of Artificial Sequence Synthetic primer”
aaggatctaggttgtgc

SEQ ID NO: 84 - DNA, 16 bp, Artificial Sequence
  source: /note=“Description of Artificial Sequence Synthetic primer”
cactgcactccagtct

SEQ ID NO: 85 - DNA, 17 bp, Artificial Sequence
  source: /note=“Description of Artificial Sequence Synthetic primer”
ctggagctatggtcagt

SEQ ID NO: 86 - DNA, 18 bp, Artificial Sequence
  source: /note=“Description of Artificial Sequence Synthetic primer”
agatagctgggactttag

SEQ ID NO: 87 - DNA, 20 bp, Artificial Sequence
  source: /note=“Description of Artificial Sequence Synthetic primer”
gttggatgattgatgagaac

SEQ ID NO: 88 - DNA, 15 bp, Artificial Sequence
  source: /note=“Description of Artificial Sequence Synthetic primer”
caaccaccacactgg

SEQ ID NO: 89 - DNA, 17 bp, Artificial Sequence
  source: /note=“Description of Artificial Sequence Synthetic primer”
gcgacaagaacaagact

SEQ ID NO: 90 - DNA, 15 bp, Artificial Sequence
  source: /note=“Description of Artificial Sequence Synthetic primer”
tgggagcagtgaacg

SEQ ID NO: 91 - DNA, 17 bp, Artificial Sequence
  source: /note=“Description of Artificial Sequence Synthetic primer”
catgccagatacacacc

SEQ ID NO: 92 - DNA, 15 bp, Artificial Sequence
  source: /note=“Description of Artificial Sequence Synthetic primer”
atccccctaggccaa

SEQ ID NO: 93 - DNA, 13 bp, Artificial Sequence
  source: /note=“Description of Artificial Sequence Synthetic primer”
aatgccgccctcg

SEQ ID NO: 94 - DNA, 19 bp, Artificial Sequence
  source: /note=“Description of Artificial Sequence Synthetic primer”
tacaacagtgagaccatag

SEQ ID NO: 95 - DNA, 15 bp, Artificial Sequence
  source: /note=“Description of Artificial Sequence Synthetic primer”
tagctccccctactg

SEQ ID NO: 96 - DNA, 20 bp, Artificial Sequence
  source: /note=“Description of Artificial Sequence Synthetic primer”
ctgctccttttcttgaaaca

SEQ ID NO: 97 - DNA, 15 bp, Artificial Sequence
  source: /note=“Description of Artificial Sequence Synthetic primer”
ggcccaaagcagtga

SEQ ID NO: 98 - DNA, 18 bp, Artificial Sequence
  source: /note=“Description of Artificial Sequence Synthetic primer”
agctggaaagttagcttg

SEQ ID NO: 99 - DNA, 18 bp, Artificial Sequence
  source: /note=“Description of Artificial Sequence Synthetic primer”
ggtgatagctgaagtcat

SEQ ID NO: 100 - DNA, 15 bp, Artificial Sequence
  source: /note=“Description of Artificial Sequence Synthetic primer”
aagtccaggttgccc

SEQ ID NO: 101 - DNA, 16 bp, Artificial Sequence
  source: /note=“Description of Artificial Sequence Synthetic primer”
gatgttcctgagggga

SEQ ID NO: 102 - DNA, 19 bp, Artificial Sequence
  source: /note=“Description of Artificial Sequence Synthetic primer”
acactgaagttgtgcatgt

SEQ ID NO: 103 - DNA, 20 bp, Artificial Sequence
  source: /note=“Description of Artificial Sequence Synthetic primer”
gaaatttgctcagtgctagt

SEQ ID NO: 104 - DNA, 15 bp, Artificial Sequence
  source: /note=“Description of Artificial Sequence Synthetic primer”
ggagaggagtctgag

SEQ ID NO: 105 - DNA, 15 bp, Artificial Sequence
  source: /note=“Description of Artificial Sequence Synthetic primer”
tccctgtagtgggga

SEQ ID NO: 106 - DNA, 18 bp, Artificial Sequence
  source: /note=“Description of Artificial Sequence Synthetic primer”
gtcaggaagaatcagatc

SEQ ID NO: 107 - DNA, 15 bp, Artificial Sequence
  source: /note=“Description of Artificial Sequence Synthetic primer”
tctcgaactcccgac

SEQ ID NO: 108 - DNA, 17 bp, Artificial Sequence
  source: /note=“Description of Artificial Sequence Synthetic primer”
gaccaacctaaatctgg

SEQ ID NO: 109 - DNA, 17 bp, Artificial Sequence
  source: /note=“Description of Artificial Sequence Synthetic primer”
ccagtgttcttctaggg

SEQ ID NO: 110 - DNA, 15 bp, Artificial Sequence
  source: /note=“Description of Artificial Sequence Synthetic primer”
ccgtccactcttgtc

SEQ ID NO: 111 - DNA, 24 bp, Artificial Sequence
  source: /note=“Description of Artificial Sequence Synthetic primer”
taagagacacaaaaggtattatct

SEQ ID NO: 112 - DNA, 15 bp, Artificial Sequence
  source: /note=“Description of Artificial Sequence Synthetic primer”
cttcactcgcttgcc

SEQ ID NO: 113 - DNA, 13 bp, Artificial Sequence
  source: /note=“Description of Artificial Sequence Synthetic primer”
gcgtgagccaccg

SEQ ID NO: 114 - DNA, 17 bp, Artificial Sequence
  source: /note=“Description of Artificial Sequence Synthetic primer”
ccgaaggtcatcaactc

SEQ ID NO: 115 - DNA, 17 bp, Artificial Sequence
  source: /note=“Description of Artificial Sequence Synthetic primer”
ccaagattgattgcacc

SEQ ID NO: 116 - DNA, 17 bp, Artificial Sequence
  source: /note=“Description of Artificial Sequence Synthetic primer”
gtctaggtctagttctg

SEQ ID NO: 117 - DNA, 19 bp, Artificial Sequence
  source: /note=“Description of Artificial Sequence Synthetic primer”
aagattaccctggttcatg

SEQ ID NO: 118 - DNA, 17 bp, Artificial Sequence
  source: /note=“Description of Artificial Sequence Synthetic primer”
attacaggtgtgcacca

SEQ ID NO: 119 - DNA, 17 bp, Artificial Sequence
  source: /note=“Description of Artificial Sequence Synthetic primer”
gtgtgtatctggcatga

SEQ ID NO: 120 - DNA, 16 bp, Artificial Sequence
  source: /note=“Description of Artificial Sequence Synthetic primer”
cagaactgagacccac

SEQ ID NO: 121 - DNA, 15 bp, Artificial Sequence
  source: /note=“Description of Artificial Sequence Synthetic primer”
ggcgggcataatgga

SEQ ID NO: 122 - DNA, 24 bp, Artificial Sequence
  source: /note=“Description of Artificial Sequence Synthetic primer”
tacataccataagaattttgtgtc

SEQ ID NO: 123 - DNA, 15 bp, Artificial Sequence
  source: /note=“Description of Artificial Sequence Synthetic primer”
tcactggccccagtt

SEQ ID NO: 124 - DNA, 17 bp, Artificial Sequence
  source: /note=“Description of Artificial Sequence Synthetic primer”
gcaggaagacatggact

SEQ ID NO: 125 - DNA, 16 bp, Artificial Sequence
  source: /note=“Description of Artificial Sequence Synthetic primer”
ctcttcctctaacccg

SEQ ID NO: 126 - protein, 1342 aa, Homo sapiens; identical to SEQ ID NO: 1

SEQ ID NO: 127 - protein, 1339 aa, Mus musculus
MSAIGTLQVLGFLLSLARGSEMGNSQAVCPGTLNGLSVTGDADNQYQTLYKLYEKCEVVMGNLEIVLTGHNADLSFLQWIREVTGYVLVAMNEFSVLPLPNLRVVRGTQVYDGKFAIFVMLNYNTNSSHALRQLRFTQLTEILLGGVYIEKNDKLCHMDTIDWRDIVRVPDAEIVVKNNGGNCPPCHEVCKGRCWGPGPEDCQILTKTICAPQCNGRCFGPNPNQCCHDECAGGCSGPQDTDCFACRHFNDSGACVPRCPAPLVYNKLTFQLEPNPHIKYQYGGVCVASCPHNFVVDQTFCVRACPADKMEVDKNGLKMCEPCRGLCPKACEGTGSGSRYQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRVYISANQQLCYHHSLNWTRLLRGPAEERLDIKYNRPLGECVAEGKVCDPLCSSGGCWGPGPGQCLSCRNYSREGVCVTHCNVLQGEPREFVHEAHCFSCHPECQPMEGTSTCNGSGSDACARCAHFRDGPHCVNSCPHGILGAKGPIYKYPDAQNECRPCHENCTQGCKGPELQDCLGQAEVLMSKPHLVIAVTVGLTVIFLILGGSFLYWRGRRIQNKRAMRRYLERGESIEPLDPSEKANKVLARIFKETELRKLKVLGSGVFGTVHKGIWIPEGESIKIPVCIKVIEDKSGRQSFQAVTDHMLAVGSLDHAHIVRLLGLCPGSSLQLVTQYLPLGSLLDHVRQHRETLGPQLLLNWGVQIAKGMYYLEEHSMVHRDLALRNVMLKSPSQVQVADFGVADLLPPDDKQLLHSEAKTPIKWMALESIHFGKYTHQSDVWSYGVTVWELMTFGAEPYAGLRLAEIPDLLEKGERLAQPQICTIDVYMVMVKCWMIDENIRPTFKELANEFTRMARDPPRYLVIKRASGPGIPPAAEPSALSTKELQDAELEPDLDLDLDVEVEEEGLATTLGSALSLPTGTLTRPRGSQSLLSPSSGYMPMNQSNLGEACLDSAVLGGREQFSRPISLHPIPRGRQTSESSEGHVTGSEAELQERVSMCRSRSRSRSPRPRGDSAYHSQRHSLLTPVTPLSPPGLEEEDGNGYVMPDTHLRGTSSSREGTLSSVGLSSVLGTEEEDEDEEYEYMNRKRRGSPARPPRPGSLEELGYEYMDVGSDLSASLGSTQSCPLHPMAIVPSAGTTPDEDYEYMNRRRGAGGSGGDYAAMGACPAAEQGYEEMRAFQGPGHQAPHVRYARLKTLRSLEATDSAFDNPDYWHSRLFPKANAQRI

SEQ ID NO: 128 - protein, 1339 aa, Rattus norvegicus
MRATGTLQVLCFLLSLARGSEMGNSQAVCPGTLNGLSVTGDADNQYQTLYKLYEKCEVVMGNLEIVLTGHNADLSFLQWIREVTGYVLVAMNEFSVLPLPNLRVVRGTQVYDGKFAIFVMLNYNTNSSHALRQLKFTQLTEILSGGVYIEKNDKLCHMDTIDWRDIVRVRGAEIVVKNNGANCPPCHEVCKGRCWGPGPDDCQILTKTICAPQCNGRCFGPNPNQCCHDECAGGCSGPQDTDCFACRRFNDSGACVPRCPEPLVYNKLTFQLEPNPHTKYQYGGVCVASCPHNFVVDQTFCVRACPPDKMEVDKHGLKMCEPCGGLCPKACEGTGSGSRYQTVDSSNIDGFVNCTKILGNLDFLITGLNVDPWHKIPALDPEKLNVFRTVREITGYLNIQSWPPHMHNFSVFSNLTTIGGRSLYNRGFSLLIMKNLNVTSLGFRSLKEISAGRVYISANQQLCYHHSLNWTRLLRGPSEERLDIKYDRPLGECLAEGKVCDPLCSSGGCWGPGPGQCLSCRNYSREGVCVTHCNFLQGEPREFVHEAQCFSCHPECLPMEGTSTCNGSGSDACARCAHFRDGPHCVNSCPHGILGAKGPIYKYPDAQNECRPCHENCTQGCNGPELQDCLGQAEVLMSKPHLVIAVTVGLAVILMILGGSFLYWRGRRIQNKRAMRRYLERGESIEPLDPSEKANKVLARIFKETELRKLKVLGSGVFGTVHKGIWIPEGESIKIPVCIKVIEDKSGRQSFQAVTDHMLAVGSLDHAHIVRLLGLCPGSSLQLVTQYLPLGSLLDHVKQHRETLGPQLLLNWGVQIAKGMYYLEEHSMVHRDLALRNVMLKSPSQVQVADFGVADLLPPDDKQLLHSEAKTPIKWMALESIHFGKYTHQSDVWSYGVTVWELMTFGAEPYAGLRLAEIPDLLEKGERLAQPQICTIDVYMVMVKCWMIDENIRPTFKELANEFTRMARDPPRYLVIKRASGPGTPPAAEPSVLTTKELQEAELEPELDLDLDLEAEEEGLATSLGSALSLPTGTLTRPRGSQSLLSPSSGYMPMNQSSLGEACLDSAVLGGREQFSRPISLHPIPRGRPASESSEGHVTGSEAELQEKVSVCRSRSRSRSPRPRGDSAYHSQRHSLLTPVTPLSPPGLEEEDGNGYVMPDTHLRGASSSREGTLSSVGLSSVLGTEEEDEDEEYEYMNRKRRGSPPRPPRPGSLEELGYEYMDVGSDLSASLGSTQSCPLHPMAIVPSAGTTPDEDYEYMNRRRGAGGAGGDYAAMGACPAAEQGYEEMRAFQGPGHHAPHVRYARLKTLRSLEATDSAFDNPDYWHSRLFPKANAQRT

SEQ ID NO: 129 - protein, 1336 aa, Bos taurus
MRVNRALQVLGFLLSLARGSEVGNSQAVCPGTLNGLSVTGDAENQYQTLHKLYEKCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNEKLCHMDTIDWRDIVRDRDAEIVVKNNGKTCPPCHEACKGRCWGPGPEDCQTLTKTICAPQCNGHCFGPNPNQCCHDECAGGCSGPQNTDCFACRLFNDSGACVRQCPQPLVYNKLTFQLEPNPHTKYQYGGVCVASCPHNFVVDQTSCVRACPPDKMEVDKNGLKICEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTRLLRGPSEERLDIKHNRPRRDCVAEGKVCDPLCSGGCWGPGPGQCLSCRNYSRGGVCVTHCNFLNGEPREFAHEAECFSCHQECQPMEGTVTCNGSGSDACAQCAHFRDGPHCVSSCPFGVLGAKGPIYKYPDAQNECRPCHENCTQGCKGPELQDCLGQLLPLISKTHLAMALTVVVGLAVTFLILGSTFLYWRGRKIQNKRAMRRYLERGESVEPLDPSEKANKVLARVFKETELRKLKVLGSGIFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYNEAKTPIKWMALESIHFGKYTHQSDVWSYGVTVWELMTFGAEPYAGLRLAEIPDLLEKGERLAQPQICTIDVYMVMVKCWMIDENIRPTFKELANEFTRMARDPPRYLVIKRESGPGITPGAEPPPLTNKELEEVELEPELDLDLELEAEEENLATTLGSALSLPIGTLNRPRGSQSLVSPSSGYMPMNQGNLGEVGQESAVFGGNERYPRPASLHPMPRGRLASESSEGHVTGSEAELQEKVSMCRSQSRSPRPRGDSAYHSQRHSLLTPVTPQSPPGLEEEDVNGYVMPDTHIKGTSSREGTLSSVGLSSVLGTEDDDDEEYEYMNRRRRCSPSRPPRPSSLEELGYEYMDVGSDLSASLGSTQSCPLNPVPNMPNASTTPDEDYEYMNRRRGGGGPGGDYAAMDACPASEQGYEEMRAFQGPVLHGPQVHYARLKTLRSLEATDSAFDNPDYWHSRLFPKANAQRI

SEQ ID NO: 130 - protein, 1342 aa, Pan troglodytes
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSETELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPIPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 131 - protein, 1536 aa, Canis lupus
MGPDHPEVMTGEEAKSWAPARGAAKGLSPRAPLISGRCEPEPRLPVVTLPPGAQLLRGETSAPGGPGARAGSEPRPGGPWKGSRLGAEAARTLSPRSCSLCGGNRRSPALLRIRLALRLGGPPRRQAPRAVLPPTGARVGAAEGPAGLGGRAPVPTQPRARTRERPPEPPRRRCRSLAAQVAPLGCPSRGPRDGSRGASAASAGLMRATAPLQVLGFLLSLVRASYVGNSQAVCPGTLNGLSVTGDAENQYQTLYKLYERCEVVMGNLEIVLTGHNADLSFLQWIREVTGYVLVAMNEFPTLPLPNLRVVRGTQVYDGKFAIFVMLNYNTNSSHALRQLRFTQLTEILAGGVYIEKNDKLCHMDTIDWRDIVRDRDAEIVVKDNGRSCPPCHETCKGRCWGPRPEDCQTLTKTICAPQCNGHCFGPNPNQCCHDECAGGCSGPQDTDCFACRLFNDSGACVRQCPQPLVYNKLTFQLEPNPHTKYQYGGVCVASCPRKCLRRGTMIMEVDKNGSKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNITSLGLRSLKEISAGRIYISANKQLCYHHSLNWTRLLRGPPEERLDIKHNRPRRDCVAEGKVCDPLCSSGGCWGPGPGQCLSCRNYSRGGVCVTHCNFLNGEPREFAHEAECFSCHPECQPMEGTATCNGSGSDACAQCAHFRDGPHCVSSCPNGVLGAKGPIYKYPDTHNECRPCHENCTQGCKGPELQDCLGQTLALISKTHLAVGLTVVVGLAVIFLILGGTLLYWRGRRIQNKRAMRRYLERGESIEPLDPSEKANKVLARIFKETELRKLKVLGSGVFGTVHKGVWIPEGESIKIPVCIKVIEDKSGRQSFQDVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLHSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIPPGAEPPALTNKELEEVELEPELELDLDLETEEDGLAATLNSALGLPVGTLNRPRGSQSLLSPSSGYMPMNQGNLGDTCQESAICGTGERCPRPASLHPMPRGRLASESSEGHVTGSEAELQEKASMCRSRSRSPRPRGDSAYHSQRHSLLTPVTPLSPPGLEEEDVNGYVMPDAHLKGTPSSREGTLSSVGISSVLGTEEEEEDEEYEYMNRRRRHSPPRHPRPSSLEELGYEYMDVGSDLSASLGSTQSCPLNPVPLMPAAGTTPDEDYEYMNRRHAGGAPGGDYAAMGACPAAEQGYEEMRAFQGPGNHAPHVHCARLKPLRSLEATDSAFDNPDYWHSRLFPKADAQRT

SEQ ID NO: 132 - protein, 20 aa, Homo sapiens
MRANDALQVLGLLFSLARGS

SEQ ID NO: 133 - protein, 21 aa, Homo sapiens
YKLYERCEVVMGNLEIVLTGH

SEQ ID NO: 134 - protein, 21 aa, Homo sapiens
PNLRVVRGTQVYDGKFAIFVM

SEQ ID NO: 135 - protein, 21 aa, Homo sapiens
GRSCPPCHEVCKGRCWGPGSE

SEQ ID NO: 136 - protein, 21 aa, Homo sapiens
GPNPNQCCHDECAGGCSGPQD

SEQ ID NO: 137 - protein, 21 aa, Homo sapiens
NDSGACVPRCPQPLVYNKLTF

SEQ ID NO: 138 - protein, 21 aa, Homo sapiens
YQYGGVCVASCPHNFVVDQTS

SEQ ID NO: 139 - protein, 21 aa, Homo sapiens
MEVDKNGLKMCEPCGGLCPKA

SEQ ID NO: 140 - protein, 21 aa, Homo sapiens
GDPWHKIPALDPEKLNVFRTV

SEQ ID NO: 141 - protein, 21 aa, Homo sapiens
QSWPPHMHNFSVFSNLTTIGG

SEQ ID NO: 142 - protein, 21 aa, Homo sapiens
LLIMKNLNVTSLGFRSLKEIS

SEQ ID NO: 143 - protein, 21 aa, Homo sapiens
ERLDIKHNRPRRDCVAEGKVC

SEQ ID NO: 144 - protein, 21 aa, Homo sapiens
LARIFKETELRKLKVLGSGVF

SEQ ID NO: 145 - protein, 21 aa, Homo sapiens
RQHRGALGPQLLLNWGVQIAK

SEQ ID NO: 146 - protein, 21 aa, Homo sapiens
VLLKSPSQVQVADFGVADLLP

SEQ ID NO: 147 - protein, 21 aa, Homo sapiens
VPDLLEKGERLAQPQICTIDV

SEQ ID NO: 148 - protein, 21 aa, Homo sapiens
ALSLPVGTLNRPRGSQSLLSP

SEQ ID NO: 149 - protein, 21 aa, Homo sapiens
RPVSLHPMPRGCLASESSEGH

SEQ ID NO: 150 - protein, 21 aa, Homo sapiens
VMPDTHLKGTPSSREGTLSSV

SEQ ID NO: 151 - protein, 21 aa, Homo sapiens
EEDEDEEYEYMNRRRRHSPPH

SEQ ID NO: 152 - protein, 20 aa, Mus musculus
MSAIGTLQVLGFLLSLARGS

SEQ ID NO: 153 - protein, 21 aa, Mus musculus
YKLYEKCEVVMGNLEIVLTGH

SEQ ID NO: 154 - protein, 21 aa, Mus musculus
PNLRVVRGTQVYDGKFAIFVM

SEQ ID NO: 155 - protein, 21 aa, Mus musculus
GGNCPPCHEVCKGRCWGPGPE

SEQ ID NO: 156 - protein, 21 aa, Mus musculus
GPNPNQCCHDECAGGCSGPQD

SEQ ID NO: 157 - protein, 21 aa, Mus musculus
NDSGACVPRCPAPLVYNKLTF

SEQ ID NO: 158 - protein, 21 aa, Mus musculus
YQYGGVCVASCPHNFVVDQTF

SEQ ID NO: 159 - protein, 21 aa, Mus musculus
MEVDKNGLKMCEPCRGLCPKA

SEQ ID NO: 160 - protein, 21 aa, Mus musculus
GDPWHKIPALDPEKLNVFRTV

SEQ ID NO: 161 - protein, 21 aa, Mus musculus
QSWPPHMHNFSVFSNLTTIGG

SEQ ID NO: 162 - protein, 21 aa, Mus musculus
LLIMKNLNVTSLGFRSLKEIS

SEQ ID NO: 163 - protein, 21 aa, Mus musculus
ERLDIKYNRPLGECVAEGKVC

SEQ ID NO: 164 - protein, 21 aa, Mus musculus
LARIFKETELRKLKVLGSGVF

SEQ ID NO: 165 - protein, 21 aa, Mus musculus
RQHRETLGPQLLLNWGVQIAK

SEQ ID NO: 166 - protein, 21 aa, Mus musculus
VMLKSPSQVQVADFGVADLLP

SEQ ID NO: 167 - protein, 21 aa, Mus musculus
IPDLLEKGERLAQPQICTIDV

SEQ ID NO: 168 - protein, 21 aa, Mus musculus
ALSLPTGTLTRPRGSQSLLSP

SEQ ID NO: 169 - protein, 21 aa, Mus musculus
RPISLHPIPRGRQTSESSEGH

SEQ ID NO: 170 - protein, 21 aa, Mus musculus
VMPDTHLRGTSSSREGTLSSV

SEQ ID NO: 171 - protein, 21 aa, Mus musculus
EEDEDEEYEYMNRKRRGSPAR

SEQ ID NO: 172 - protein, 20 aa, Rattus norvegicus
MRATGTLQVLCFLLSLARGS

SEQ ID NO: 173 - protein, 21 aa, Rattus norvegicus
YKLYEKCEVVMGNLEIVLTGH

SEQ ID NO: 174 - protein, 21 aa, Rattus norvegicus
PNLRVVRGTQVYDGKFAIFVM

SEQ ID NO: 175 - protein, 21 aa, Rattus norvegicus
GANCPPCHEVCKGRCWGPGPD

SEQ ID NO: 176 - protein, 21 aa, Rattus norvegicus
GPNPNQCCHDECAGGCSGPQD

SEQ ID NO: 177 - protein, 21 aa, Rattus norvegicus
NDSGACVPRCPEPLVYNKLTF

SEQ ID NO: 178 - protein, 21 aa, Rattus norvegicus
YQYGGVCVASCPHNFVVDQTF

SEQ ID NO: 179 - protein, 21 aa, Rattus norvegicus
MEVDKHGLKMCEPCGGLCPKA

SEQ ID NO: 180 - protein, 21 aa, Rattus norvegicus
VDPWHKIPALDPEKLNVFRTV

SEQ ID NO: 181 - protein, 21 aa, Rattus norvegicus
QSWPPHMHNFSVFSNLTTIGG

SEQ ID NO: 182 - protein, 21 aa, Rattus norvegicus
LLIMKNLNVTSLGFRSLKEIS

SEQ ID NO: 183 - protein, 21 aa, Rattus norvegicus
ERLDIKYDRPLGECLAEGKVC

SEQ ID NO: 184 - protein, 21 aa, Rattus norvegicus
LARIFKETELRKLKVLGSGVF

SEQ ID NO: 185 - protein, 21 aa, Rattus norvegicus
KQHRETLGPQLLLNWGVQIAK

SEQ ID NO: 186 - protein, 21 aa, Rattus norvegicus
VMLKSPSQVQVADFGVADLLP

SEQ ID NO: 187 - protein, 21 aa, Rattus norvegicus
IPDLLEKGERLAQPQICTIDV

SEQ ID NO: 188 - protein, 21 aa, Rattus norvegicus
ALSLPTGTLTRPRGSQSLLSP

SEQ ID NO: 189 - protein, 21 aa, Rattus norvegicus
RPISLHPIPRGRPASESSEGH

SEQ ID NO: 190 - protein, 21 aa, Rattus norvegicus
VMPDTHLRGASSSREGTLSSV

SEQ ID NO: 191 - protein, 21 aa, Rattus norvegicus
EEDEDEEYEYMNRKRRGSPPR

SEQ ID NO: 192 - protein, 20 aa, Bos taurus
MRVNRALQVLGFLLSLARGS

SEQ ID NO: 193 - protein, 21 aa, Bos taurus
HKLYEKCEVVMGNLEIVLTGH

SEQ ID NO: 194 - protein, 21 aa, Bos taurus
PNLRVVRGTQVYDGKFAIFVM

SEQ ID NO: 195 - protein, 21 aa, Bos taurus
GKTCPPCHEACKGRCWGPGPE

SEQ ID NO: 196 - protein, 21 aa, Bos taurus
GPNPNQCCHDECAGGCSGPQN

SEQ ID NO: 197 - protein, 21 aa, Bos taurus
NDSGACVRQCPQPLVYNKLTF

SEQ ID NO: 198 - protein, 21 aa, Bos taurus
YQYGGVCVASCPHNFVVDQTS

SEQ ID NO: 199 - protein, 21 aa, Bos taurus
MEVDKNGLKICEPCGGLCPKA

SEQ ID NO: 200 - protein, 21 aa, Bos taurus
GDPWHKIPALDPEKLNVFRTV

SEQ ID NO: 201 - protein, 21 aa, Bos taurus
QSWPPHMHNFSVFSNLTTIGG

SEQ ID NO: 202 - protein, 21 aa, Bos taurus
LLIMKNLNVTSLGFRSLKEIS

SEQ ID NO: 203 - protein, 21 aa, Bos taurus
ERLDIKHNRPRRDCVAEGKVC

SEQ ID NO: 204 - protein, 21 aa, Bos taurus
LARVFKETELRKLKVLGSGIF

SEQ ID NO: 205 - protein, 21 aa, Bos taurus
RQHRGALGPQLLLNWGVQIAK

SEQ ID NO: 206 - protein, 21 aa, Bos taurus
VLLKSPSQVQVADFGVADLLP

SEQ ID NO: 207 - protein, 21 aa, Bos taurus
IPDLLEKGERLAQPQICTIDV

SEQ ID NO: 208 - protein, 21 aa, Bos taurus
ALSLPIGTLNRPRGSQSLVSP

SEQ ID NO: 209 - protein, 21 aa, Bos taurus
RPASLHPMPRGRLASESSEGH

SEQ ID NO: 210 - protein, 20 aa, Bos taurus
VMPDTHIKGTSSREGTLSSV

SEQ ID NO: 211 - protein, 20 aa, Bos taurus
DDDDEEYEYMNRRRRCSPSR

SEQ ID NO: 212 - protein, 21 aa, Pan troglodytes
PNLRVVRGTQVYDGKFAIFVM

SEQ ID NO: 213 - protein, 21 aa, Pan troglodytes
GRSCPPCHEVCKGRCWGPGSE

SEQ ID NO: 214 - protein, 21 aa, Pan troglodytes
GPNPNQCCHDECAGGCSGPQD

SEQ ID NO: 215 - protein, 21 aa, Pan troglodytes
NDSGACVPRCPQPLVYNKLTF

SEQ ID NO: 216 - protein, 21 aa, Pan troglodytes
YQYGGVCVASCPHNFVVDQTS

SEQ ID NO: 217 - protein, 21 aa, Pan troglodytes
MEVDKNGLKMCEPCGGLCPKA

SEQ ID NO: 218 - protein, 21 aa, Pan troglodytes
GDPWHKIPALDPEKLNVFRTV

SEQ ID NO: 219 - protein, 21 aa, Pan troglodytes
QSWPPHMHNFSVFSNLTTIGG

SEQ ID NO: 220 - protein, 21 aa, Pan troglodytes
LLIMKNLNVTSLGFRSLKEIS

SEQ ID NO: 221 - protein, 21 aa, Pan troglodytes
ERLDIKHNRPRRDCVAEGKVC

SEQ ID NO: 222 - protein, 21 aa, Pan troglodytes
LARIFKETELRKLKVLGSGVF

SEQ ID NO: 223 - protein, 21 aa, Pan troglodytes
RQHRGALGPQLLLNWGVQIAK

SEQ ID NO: 224 - protein, 21 aa, Pan troglodytes
VLLKSPSQVQVADFGVADLLP

SEQ ID NO: 225 - protein, 21 aa, Pan troglodytes
VPDLLEKGERLAQPQICTIDV

SEQ ID NO: 226 - protein, 21 aa, Pan troglodytes
ALSLPVGTLNRPRGSQSLLSP

SEQ ID NO: 227 - protein, 21 aa, Pan troglodytes
RPVSLHPMPRGCLASESSEGH

SEQ ID NO: 228 - protein, 21 aa, Pan troglodytes
VMPDTHLKGTPSSREGTLSSV

SEQ ID NO: 229 - protein, 21 aa, Pan troglodytes
EEDEDEEYEYMNRRRRHSPPH

SEQ ID NO: 230 - DNA, 4029 bp, Homo sapiens
  CDS (1)..(4026)
atgagggcgaacgacgctctgcaggtgctgggcttgcttttcagcctggcccggggctccgaggtgggcaactctcaggcagtgtgtcctgggactctgaatggcctgagtgtgaccggcgatgctgagaaccaataccagacactgtacaagctctacgagaggtgtgaggtggtgatggggaaccttgagattgtgctcacgggacacaatgccgacctctccttcctgcagtggattcgagaagtgacaggctatgtcctcgtggccatgaatgaattctctactctaccattgcccaacctccgcgtggtgcgagggacccaggtctacgatgggaagtttgccatcttcgtcatgttgaactataacaccaactccagccacgctctgcgccagctccgcttgactcagctcaccgagattctgtcagggggtgtttatattgagaagaacgataagctttgtcacatggacacaattgactggagggacatcgtgagggaccgagatgctgagatagtggtgaaggacaatggcagaagctgtcccccctgtcatgaggtttgcaaggggcgatgctggggtcctggatcagaagactgccagacattgaccaagaccatctgtgctcctcagtgtaatggtcactgctttgggcccaaccccaaccagtgctgccatgatgagtgtgccgggggctgctcaggccctcaggacacagactgctttgcctgccggcacttcaatgacagtggagcctgtgtacctcgctgtccacagcctcttgtctacaacaagctaactttccagctggaacccaatccccacaccaagtatcagtatggaggagtttgtgtagccagctgtccccataactttgtggtggatcaaacatcctgtgtcagggcctgtcctcctgacaagatggaagtagataaaaatgggctcaagatgtgtgagccttgtgggggactatgtcccaaagcctgtgagggaacaggctctgggagccgcttccagactgtggactcgagcaacattgatggatttgtgaactgcaccaagatcctgggcaacctggactttctgatcaccggcctcaatggagacccctggcacaagatccctgccctggacccagagaagctcaatgtcttccggacagtacgggagatcacaggttacctgaacatccagtcctggccgccccacatgcacaacttcagtgttttttccaatttgacaaccattggaggcagaagcctctacaaccggggcttctcattgttgatcatgaagaacttgaatgtcacatctctgggcttccgatccctgaaggaaattagtgctgggcgtatctatataagtgccaataggcagctctgctaccaccactctttgaactggaccaaggtgcttcgggggcctacggaagagcgactagacatcaagcataatcggccgcgcagagactgcgtggcagagggcaaagtgtgtgacccactgtgctcctctgggggatgctggggcccaggccctggtcagtgcttgtcctgtcgaaattatagccgaggaggtgtctgtgtgacccactgcaactttctgaatggggagcctcgagaatttgcccatgaggccgaatgcttctcctgccacccggaatgccaacccatggagggcactgccacatgcaatggctcgggctctgatacttgtgctcaatgtgcccattttcgagatgggccccactgtgtgagcagctgcccccatggagtcctaggtgccaagggcccaatctacaagtacccagatgttcagaatgaatgtcggccctgccatgagaactgcacccaggggtgtaaaggaccagagcttcaagactgtttaggacaaacactggtgctgatcggcaaaacccatctgacaatggctttgacagtgatagcaggattggtagtgattttcatgatgctgggcggcacttttctctactggcgtgggcgccggattcagaataaaagggctatgaggcgatacttggaacggggtgagagcatagagcctctggaccccagtgagaaggctaacaaagtcttggccagaatcttcaaagagacagagctaaggaagcttaaagtgcttggctcgggtgtctttggaactgtgcacaaaggagtgtggatccctgagggtgaatcaatcaagattccagtctgcattaaagtcattgaggacaagagtggacggcagagttttcaagctgtgacagatcatatgctggccattggcagcctggaccatgcccacattgtaaggctgctgggactatgcccagggtcatctctgcagcttgtcactcaatatttgcctctgggttctctgctggatcatgtgagacaacaccggggggcactggggccacagctgctgctcaactggggagtacaaattgccaagggaatgtactaccttgaggaacatggtatggtgcatagaaacctggctgcccgaaacgtgctactcaagtcacccagtcaggttcaggtggcagattttggtgtggctgacctgctgcctcctgatgataagcagctgctatacagtgaggccaagactccaattaagtggatggcccttgagagtatccactttgggaaatacacacaccagagtgatgtctggagctatggtgtgacagtttgggagttgatgaccttcggggcagagccctatgcagggctacgattggctgaagtaccagacctgctagagaagggggagcggttggcacagccccagatctgcacaattgatgtctacatggtgatggtcaagtgttggatgattgatgagaacattcgcccaacctttaaagaactagccaatgagttcaccaggatggcccgagacccaccacggtatctggtcataaagagagagagtgggcctggaatagcccctgggccagagccccatggtctgacaaacaagaagctagaggaagtagagctggagccagaactagacctagacctagacttggaagcagaggaggacaacctggcaaccaccacactgggctccgccctcagcctaccagttggaacacttaatcggccacgtgggagccagagccttttaagtccatcatctggatacatgcccatgaaccagggtaatcttggggagtcttgccaggagtctgcagtttctgggagcagtgaacggtgcccccgtccagtctctctacacccaatgccacggggatgcctggcatcagagtcatcagaggggcatgtaacaggctctgaggctgagctccaggagaaagtgtcaatgtgtaggagccggagcaggagccggagcccacggccacgcggagatagcgcctaccattcccagcgccacagtctgctgactcctgttaccccactctccccacccgggttagaggaagaggatgtcaacggttatgtcatgccagatacacacctcaaaggtactccctcctcccgggaaggcaccctttcttcagtgggtctcagttctgtcctgggtactgaagaagaagatgaagatgaggagtatgaatacatgaaccggaggagaaggcacagtccacctcatccccctaggccaagttcccttgaggagctgggttatgagtacatggatgtggggtcagacctcagtgcctctctgggcagcacacagagttgcccactccaccctgtacccatcatgcccactgcaggcacaactccagatgaagactatgaatatatgaatcggcaacgagatggaggtggtcctgggggtgattatgcagccatgggggcctgcccagcatctgagcaagggtatgaagagatgagagcttttcaggggcctggacatcaggccccccatgtccattatgcccgcctaaaaactctacgtagcttagaggctacagactctgcctttgataaccctgattactggcatagcaggcttttccccaaggctaatgcccagagaacgtaa

SEQ ID NO: 231 - protein, 1342 aa, Homo sapiens; identical to SEQ ID NO: 1